SEQUENCE LISTING (26 sequences)

SEQ ID NO: 1 - DNA, 1401 bp, Helichrysum umbraculigerum
  source 1..1401: mol_type = other DNA
atgaccaactcggaacttgttttcatcccatctccgggagccggccacctaccacctacggtggagctagcaaagctcctcctccaccgcgaaccacagctttcggttaccatcatcatcatgaacctccctcatgaaacaaaacccactactgaaactcgaatgtccactcctcgtctacgctttattgacatacctaaagacgagtcaacaaaagatcttatctcacgccacacattcatatccgccttccttgaacaccaaaagccacatgttcgaaacattgtccgttcaatcaccgagtctgactcggttcggttagttgggttcgtcgtagacatgttttgtattgccatgatggacgtcgcaaacgagctgggtgctccaacttatctttatttcacctcctctgccgcttcacttggcctcatgttttgcctacaggccaaacgagacgacgaggagtttgatgtgaccgagttgaaggacaaagattcggaactctccattccgtgttacaccaacccactcccagctaagttgttaccttcggtactatttgataagagaggtgggtcaaaaacatttattgacctcgctagaaagtatcgcgagtcgaggggtatagttgtaaatacttttcaagaactcgaaagctatgctattgagtatcttgcaagtagtaatgctaacgtcccaccggtgtttccggtgggggcgatactaaaccaagaaaaaaaggtaaatgatgataagacggaggagattatgacatggttaaacgagcaaccggagagttcggtggtgtttctatgcttcgggagcatgggaagcttcggtgaggatcaaattaaggaaatagcgcttgctatcgaagaaagcggacaaaggtttttgtggtcactacgtcgtcccccttcgaacgaaaataagtacccgaaagaatacgaaaattttggagaggttcttccggaaggtttccttgaacgaacatcgagtgtagggaaagtgataggatgggccccacaaatggcagtgttgtcccattcttcagttggtgggtttgtgtcacattgcggatggaactcgacactcgagagcatatggtgtggtgtaccggtagctgcgtggccattatatgcagaacaacaacttaatgcttttaaactagtggtggagttgggcttagcggtcgagattaagattgattataggagtgagaacgagattattttgacatcgaaagaaatcgagagtgggattaggaggttgatgaatgatgaagagttgaggatgaaagtgaaagagatgaaggggaatagtaggtttgcagtttcagagggtggatcttcttacgtatccattaggcgttttatcgaccttgtgatgactaaggagtaa

SEQ ID NO: 2 - DNA, 1428 bp, Helichrysum umbraculigerum
  source 1..1428: mol_type = other DNA
atgccgacctcagaacttgttttcatcccatcccccggtgtcggccacctgtcgcctaccatcgaactcgtcaatcaactcctccaccgcgaccagcgcctgtctgtcacaatcatcgtcatgaagttctctcttgaatcaaaacacgatacagaaactcctacatccactcctcgattacgcttcattgatatcccttatgacgagtccgctatggctctcattaacccgaacacgttcctctccgctttcgtcgagcacaacaaacctcatgttcgaaacattgttcgtgacatttccgagtctaactcggttcggctcgcggggtttgttgtggacatgttttgtgtagctatgacggatgtagtgaacgagtttgaaattccaacctatatttattttacctcgaccgcgaacttactcggactcatgttttaccttcaggccaagcgtgacgacgagggttttgatgtcaccgtgttgaaagactcagaatcagagtttttgtctgttccgagttatgtcaacccggttccagctaaggttttacctgatgcagttttggataagaatggtgggtctcaaatgtgtctggatcttgcaaaagggtttcgtgagtcgaagggcataatagtaaatacatttcaagaactcgaaaggcgtggaatcgagcaccttttaagtagtaacatgaacctcccacctgtgtttcctgtggggcctatattgaacttgagaaatgcgccaaacgatggtaaaacggccgatatcatgacatggttaaatgaccatccagagaactcggttgtgttcttgtgtttcggaagtatgggaagcttcgagaaagaacaagtgaaggagatagcgattgccatcgaacagagtgggcaacggtttctatggtcactccgtcgtccaacatcgctagaaaagtttgagtttccaaaggattacgagaacccggaggaggttttgccaaagggatttcttgaaaggacaaaaggtgtgggaaaggttatcgggtgggccccacaaatggcggtgttgtctcacccgtcagtgggagggttcgtgtcccactgtgggtggaactccacattggagagcatatggtgtggggtcccaatagcggcttggccactatatgcggaacaaaaaattaatgcttttcaattggtggtagagatgggaatggcagctgagattaggatcgactatcggactaatacgagaccgggtggtggtaaagagatgatggtaatggctgaagagattgagagtggtattaggaagttgatgagcgatgatgagatgagaaagaaagtgaaaggtatgaaggataaaagtagggctgctgttcttgaaggtggatcatctcacacatcaattgggattttaattgagaatttggtgagtataacgatctag

SEQ ID NO: 3 - DNA, 858 bp, Helichrysum umbraculigerum
  source 1..858: mol_type = other DNA
atggtgggtctcaaatgtttttggatcttgcaaaaaggttttcgtgagtcgaagggcataatagtaaatacatttcaagaactcgaaaggcgtggaatcgagcaccttttaagtagtaacatggacctcccacctgtgtttcctgtggggccgatattgaacttgagaaatgcgcgaaacgatggtaaaatggccgatatcatgacatggttaaatgaccagccagagaactcggttgtgttcttgtgtttcggaagtaggggaagcttcaaggaggaacaagtgaaggagatagcaattgccatcgaacaaagtgggcaacggtttctatggtcactccgtcgtccaacatcgatagaaacgtttgagtttccaaagtattacgagaacccggaggaggttttgccaaagggatttcttgaaaggacaaaaagtgtgggaaaggttatcgggtgggccccacaaatggcggtattgtctcacccgtcagtgggagggttcgtgtcccactgtgggtggaactccacattggagagcatatggtgtggggtcccaatagcggcttggccactatatgcggaacaacaaactaatgcttttcaattggtggtcgagatgggaatggcagcagagattaggatcgactatcggactaatacaccactggttggtggtaaagacatgatggtaacggctgaagagattgagagaggtattaggaagttgatgagcgatgatgagatgcgaaagaaagtgaaagacatgaaggataagagtagaggtgcagttttagagggtgggtcatctcatacatcaattgggaatttaattgatgttttggtgagtataacgatctag

SEQ ID NO: 4 - DNA, 1467 bp, Helichrysum umbraculigerum
  source 1..1467: mol_type = other DNA
atggcgaccaacaacctccatttccttctaattccccatataggtccaggccacactattcccatgatagatatggctaaacttcttgcaaaacaaccaaatgtaatggttacaatagctacaacacctcttaatatcacccgttacgggcacactctcgcagacgccatcaactcgtttcgcttctttgaggttccatttccggcagttgaggctggattacctgaaggatgtgaaagcacggataaaatcccaagtatggatctagtaccgaactttttaaccgcgattggtatgctagaacaaaagctagaagagcattttcacttgctagagcctcgtccgaattgtattatttctgataagtacatgtcgtggacgggtgattttgctgataagtatcggatccctagaattatgtttgatggaatgagctgttttaacgagttatgttacaacaatttgtatgaaaacaaggtgtttgaagggatgcatgaaacagaaccatttgttgtccctggtttacccgataaaattgagctaacacgaaaacagctcccacctgagtttaacccgagctcgattgatacaagtgagtttcgtcagcgggctagggacgctgaggtgagggcttatggagttgtgatcaatagttttgaggagttggaacaagaatatgttaatgagtataagaagttaagaaagggtaaggtttggtgtatcggcccgctgtcactgtgcaatagtgacaattcggataaagcccaaagaggaaatatagcgtcagtcgatgaagaaaaatgtttaaaatggcttgattctcatgaagccgactcagtagtttacgcttgttttggtagccttgttcgggtcaacaccccacaactaattgagcttggtttaggcctagaagcatcaaatcgcccgttcatttgggtggttagatcggttcatagagaaaaagaggtcgaggaatggctagtggaaagtggttttgaggagagaattaaagatagaggtttaataatccgaggttgggccccacaagtacttatcttgtctcacccttctattggagggtttttaacgcattgcggttggaactcgaccctagaatcagtctgtgcaggtgttccaatgatcacatggcctcaatttgcagagcaatttatcaacgagaagctaatagtgcaagtgttggggattggtgtgggtgttggagttgattctgttgtccatgtgggcgaagaagatagatctggggtgaaagtgaagagggagagtgttacgaaggctattgagaaagtcatggatgacgagattgatggaaatgagagacggaggagatcgaaagagtttggaaagatagctaataacgcgattaaagagggagggtcttcataccttaacttgactctgctaattcaggacataatgcgttatgcaaatgcagatgcttcaagctaa

SEQ ID NO: 5 - DNA, 1407 bp, Helichrysum umbraculigerum
  source 1..1407: mol_type = other DNA
atggaaaaaacacctcatatagccattgtaccaagtccaggaatgggccacttgatccctttagttgagtttgctaaaaaactaaaaaatcaccacaacatacatgcaactttcatcatcccaaatgatggacctttatctatttctcaaaaggtttttcttgattcacttcctaatggtttaaactatctcattctacctccggtaaattttgatgatttaccacaagatacccaaatcgaaactcgaattagtctaatggtaacacggtctcttgattcgctacgtgaagtgtttaagtcattagttgtggaaaaaaatatggttgctttgtttattgatctttttgggacagatgcatttgatgttgctattgaatttggtgtttcaccttatgtgttctttccatcaactgctatggctttatctttgtttctatatttgcctaaacttgatcagatggtttcatgtgagtatagggagcttcctgaaccggttcaaattccaggttgtataccggttcgtggacaagacttggttgacccggttcaagatagaaagaatgatgcatacaaatgggtgcttcataatgcaaagaagtattcaatggctaagggtatagcggtaaatagcttcaaggagttagaaggtggagctttgaatgctttgctagaagatgaaccgggtaagccaaaagtttatccggtcggaccgttagtacaaaccggttttagttgtgatgttgattcgatagagtgcttgaagtggttagatggtcagccatgtggttctgttttgtatatatcttttggaagcggtgggaccctttcatccagtcaacttaatgagttagctatgggtttggagttgagtgaacaacggttcatatgggtggttagaagcccgaacgatcaaccaaacgccacgtactttgattctcatggtcacaaagaccctcttggttttttgcccaaagggttcttggaaagaaccaaaggaattgggtttgtgatcccttcttgggctccacaagcccagatcctgagtcacagtgccacaggtggatttttaacccactgtggttggaactcaattctcgagactgtagtccatggtgtgccggtgattgcttggccactttatgccgagcaaaagatgaatgcagtgtctttaaccgagggtataaaaatggcgttaagacccacggttggtgaaaatgggattgtgggtcgcttagaggttgcgagagttgtgaagagtttactggaaggagaagaagggaaggcgattaggagtcgagttcgtgatctcaaggatgctgctgctaatgttcttagtaaagatgggtcttctacaaaaactttagatcaattggctgtacagttgaaaaaacaagaattaagctag

SEQ ID NO: 6 - DNA, 1434 bp, Helichrysum umbraculigerum
  source 1..1434: mol_type = other DNA
atgactcaaaagcaaatgcaaatgcaacctcactttctcttagtaacatatcccgcacaaggtcatattaacccgtctctccagttcgctgaacgtctcattcggttgggtgtcaaagtcaccttcacaacaactgtctctgcttaccgccgaatgagtaaagcgggcaacatctcagagtttttaaattttgctgctttttcagacggctttgatgacggtttcaacttcgaaacagacgatcatggtctcttcttaactcaattgagaagcaggggaaaagatagcttgaaagaaacaattctttcaaatgctaaaaatggaactccaattagttgtttggtttacacactcctactcccttgggctcctgaggtggcacgtggcctaaacgtgccctcagcctttctttggattcaaccagcttctgttttacgactttactattactacttcaatgggtacaatgaactcatcggtgacgattgtaatgaaccttcatggtccattcaattaccagggttaccattgctcaaaagtcatgaccttccctccttttgtctcccttcaaatccttacagtaatgtactggctctagtcaaagagcatttagatatgctggatctggaagagaagcctaaaatacttgtgaatagttttgatgagttggagagggaggcgttgaatgaaattaatggaaaactaaaaatggtcgccgtagggcctttgattccatcagcttttttggatggacaagatgcatctgacaaatcttttaggggagatttgtttgaaacatccaaagattatttggaatggatgaatacaaagcctgaagggtccattgtttacatatcttttggtagtcttttagtgttctcaaagatacaaaaggaggcaatggcacatgctttgttagagtgcgggaggccgttcttgtgggtgataagagatggagaacaaggagaacaactaagttgtattgagaaattggaacaattaggtttgatagtcccatggtgtagtcaactagaggtattatcacacccttctttaggttgttttgtgacacattgtggttggaactcgactttagagagtatagtttgtggagttcctgtggtggcatttcctcaatggacagatcagacgacaaatgcaaagcttctagaagacgtatggggaacaggggtgagagtgacaactaatgaagacggggttgttgaaagcgaggagataagaaggtgcatcgaaatggtaatgggaggccgtgatagtgaatcaacaatgagaaagaatgctaagaagtggaaggatgtgggaagagaggctatgaaagaaacaggatcttcttatatgaatctcaaggcttttattaaagaagtgaatgatggtgaatcaaccatcaaaactgaaattgtttcaactatatga

SEQ ID NO: 7 - DNA, 762 bp, Helichrysum umbraculigerum
  source 1..762: mol_type = other DNA
atgactaaaatacaacagcaacctcactttctcttagtaacatatcccgcacaaggtcatattaacccgtctctccggttcgccgaacgactcattcggttgggtgtcaaagtcaccttcacaataactgtctctgcttaccgccgaatgagtaaagcgggccacatctcagagtttttaaattttgctgttttttcagacggctttgatgacggtttcaactccaaaacagacgattatggtctcttcttaactcaattcagaagcaggggaaaagatagcttgaaagaaacaattctttcaaatgctaaaaacggaactccagttagttgtttggtttacacactcctactcccttgggctcctgaggtggcacgtggcctaaacgtgccctcagcctttctttggattcaaccagcttctgttttacgactttactattactacttcaatgggtacaatgaactcatcggcgacgattgtaacgaaccttcatggtccattcaattaccagggttaccattgctcaaaagtcgtgaccttccctccttttgtctcccttcaaatccttacgctgatgtactgactttagtcaaagagcatttagatgtgttggatttggaagagaagcctaaaatacttgtgaatagttttgatgagttggagagggaggcgttgaatgaaattgatgggaaactaaaaatggttgccgtagggcctttgattccatcagctttttttggatggacaggatgcatctga

SEQ ID NO: 8 - DNA, 1014 bp, Helichrysum umbraculigerum
  source 1..1014: mol_type = other DNA
atgggttcatggcggaattcaagaacaacgtctacaaagtttttatggttgattttaccgttgatggtggtgacggtgattataggggtaaaaaagtcaaattatgggtcgaagtataattatccttgggtttggagttcagtgattaattcttattcttcttctgcggttaaagaagatgtaacggtggtggctgaaggtcctgttgaatcatttgggttgcggtcaacggtggtcaacggtggtggtgtggtggcggaagggccgtcggaagattttggttttaattcttcttatccaccgttggctatggaagatgaaatggatgttgagctacctgctattgccaaggaagatgacttgaacgcgacgttgagtggacccgacctttttgtgtctgcaaatcaaactggcggacttcatgttgatattggaatcaacagtaagtataccagtttggataagcttgaagcccgcttaggtcaggttcgagctgcaataaaagaagccgaatcaggaaatagaacttacgatccggattatgtaccagagggtcctatgtactggcatgcagcctcatttcacaggagttatttggagatggaaaagcaatttaaggtgtttgtatatgaagaaggagaaccaccaatatttcataacggtccttgcaaaaacatatatgcaatggaaggtaactttatctaccatatggaaacaaccaagtttaggacaaaaaaccccgaaaaagctcacacgttttttctcccaatgagtgctgcaatgatggtgaggtttatctttgagcgtgatccaaatgttgaccattggcgtcctatgaagcaaacaattaaagattatgttgatcttgtgggtggtaagtacccattttggaatcgaagcttaggagccgatcactttactgttgcgtgccacgattgggtgagtaaagtcttttatcccatcattttcatgcttttactagtatttatcttcagaatgtcgactggatgctga

SEQ ID NO: 9 - DNA, 1410 bp, Helichrysum umbraculigerum
  source 1..1410: mol_type = other DNA
atgtcaaccgttgaggttgcaaagttacttgtgaatcgagatcatcgtctcttcataacattccttatcattcagcctcctagctcgggttctggctcagctatcaccacctacatcgaatcattagctgagaaagctatggaccgcatatccttcattgagctacctcaagataaaatcccaccaccacgttacccgaaatccctgccaactgcagaatcgaaagctcatccccttattttcatgattgagttcattaagtgtcactgcaaatatgttagaaacattgtatctgacatgataagtcaaccgagttcgggtcgggtagctgggttggtaatcgacatgctttgtttcagcatgatggatgtcgctaatgagttcaacattccaacctatgtatttgtcacttctaatgctgcttttcttggattttatttatatgtccagatactctctaatgatcagaaccaagacgttgttgagctgagcaaatctgataccgagatatcggttccaggttttgtaaagccggtgccaacgaaagtcttctggactgttgtccgcactaaagaaggactggactttgttttgtcatctgcccagaaacttagacaagccaaagcaatcatggttaataccttcttggagttggaaacacacgcaatcaagtcgctgtctgatgacaccagcatcccgcctgtgtatccagtgggaccgatactcaatttagaaggtggtgctggcaaaacgttcgacaatgacattagcaggtggttggacagtcaaccgccttcctcggtggtgttcttgtgctttggaagccacggatgttttgatgagatccaagtgaaggagatagcacatgctttagagcagagtggccaccgtttcttgtggtccctacgtcgacctccatcagatcaaacattaaaagttcccggtgattacgaggatccaggagtggtattaccggaaggatttcttgagcgaactgctggacgtgggaaagtaattgggtgggccccgcaggtgatggtgctggctcaccgtgcagttggaggcttcgtgtcccactgtgggtggaactcgttgttggagagtttgtggttcggcgtaccaacggcaacatggccgatctatgctgagcagcagatgaatgcgtttgaaatggtggtggagctgggactggctgtggagataacattggattataggaatgatatggatatgttcattgtcaccgcacaggagatagaaagtggtataagaaaggtgatggaggataatgaggtaagaacaaaagtgaaagagagaagtgagaagagtagagcagcagtggcggagggggggtcatcgtatgcatctgttggtcatcttattaaagaatttacaggaaacatctcctaa

SEQ ID NO: 10 - DNA, 1173 bp, Helichrysum umbraculigerum
  source 1..1173: mol_type = other DNA
atgtcatcattcatcaactttgttgaatccacaacacaacttcaaccacaattcgaacaactcatccaaacacttcttcccataactgcgataatatcggatggttttttgatgtggacacaagattccgccgaaaaattcaatatcccacgtctggttttttatgggacaaacatatttttcatgactatgtgtaacattatggcacaatttaagccacatgcggctgttaattctgatgatgaggcgtttgatgtacccggtttcaccaggtttaagttgacggctaatgattttgagccgccttttaatgaggttgaaccgaaaggttcaatgttggattttttattggagcaacaaaaggctatggttaggagccatgggttggtggttaatagtttttatgagattgaacatgagtttaatgtttattggaatcagaactatggacctaaagcttggttaatgggaccattttgtgtagctaagccatatgcatcaaacgtcatggattccgagatatcgactaaggtggtgaaaaaatcagcatggatccagtggcttgacaggaagcttgcagcgaacgagccagtgttatacatctcatttggaacacaggcagaggcgtctatggagcacttacacgaggtcgctattggtttggaacgatcaaatgtaagcttcatttgggtggtaaaagcgaagcagatgcaattaattggagcagggtttgaagagagggtgaaggggagaggaaaagtggtgacagaatgggtggatcagatggaaatcttgaaacatgaaattgtaagcgggtttttaagtcattgtgggtggaactcactgctagagagtatgtgtgtgggtgtgccggtgcttgcaatgccgttgatggcggatcaactcttaaatgcaaggttggttgtggaggagattgggatggggctacggttgtggccgaggggtatggtggcacgtgggatagttggggcggaggaagtcgagaaaatggtggtggagttgatggaaggggaaggtgggagaagggtgcggaaaagggtcatcgaggttagagaaatggcatatggtgcgatgaaggaaggagggtcatcatcgaggacattagactcgttgattgatcatgtttgtgaagcctttcataagacggtttaa

SEQ ID NO: 11 - DNA, 1380 bp, Helichrysum umbraculigerum
  source 1..1380: mol_type = other DNA
atggggagcttgaagaaaggtgcacatatactaatattcccattcccagcacaaggtcatatgctcccactcctagacctaactcaccacctagccaccaatgggttaaccataaccatattagtcacacccaaaaacctaccaatcttgaacccacttttatcttcatctccaaacatccaaccactagtcttccctttcccacctcacccaagacttccaccacatgttgaaaatgttaaagacataggtaaccatgcaaatgtcccaatcacaaactcactagccaaattacaagaccaaataatccagtggtttaactcccaccataaccctcctgttgccatcatctcagatttctttcttggatggacccaacaccttgcaaacaaacttggtatccctcgtgtcgggtttttttcttctggtgcttacttgactgctgttcttgattatgtttgtcataatattaaaactgttaggtctcaagaggagactgtttttcatgacttgccaaattctccttgttttaaattcgagcatcttccgggtttggcccagatttataaagagtccgacccggaatgggaattggttcttgatggtcatattgcgaatgggttaagttggggttggattgtgaatacttttgatgggttggagtctcggtatatggagtatctgacaaagaaaatgggtgtcggacgggtttttggtgtcgggccagttaatttgttaaacgggtcggatcccatgacccgtgggaaatcggaatccgggtctgattccggtgtgttgaactggctcgatggaaaacccgatgggtcggttttgtatgtgtgttttggaagtcaaaagtttcttactaatgaccaaatggagggattgtcaattgggcttgaacaaagtggggtccattatgtttgggttgtgaaagacgaacaaggtgatgcaattaggtccgggtcgggtagaggactagtggtaacgggttgggccccgcaagtttcaatattgggtcatggagcggtgggtgggtttttgagtcattgcgggtggaactctgttttggaagcaattgtaaatggagttatgatattggcttggccaatggaggctgatcaatttgttaatgctaagttgttagtggatgaccatggtataggggtgtgggtttgtgaggggccgaatacggttcctgattcaaccgagttggctcgtaaaattggtgagtcaatgagtacggataagagtgagaaggtaaaggcgaaagaaatgaaaaacaaagcaaatgaagcagttaaagaaggtgggagctcatcaatggaattaagcaggcttgttaaggagctgtctaactttgagacaaatgggccatga

SEQ ID NO: 12 - DNA, 1611 bp, Helichrysum umbraculigerum
  source 1..1611: mol_type = other DNA
atggatacccaaacacaagtcaagaaacaaaaacttgaaaccatggaacataaaacatcatccgccgaaatcttcgtgctaccattttttggtacgggtcatataaacccagcaatggagctttgccggaacatttcatcacataattacaaaactaccctcatcatcccttcacatctttcttcatctattccttctcccttttcttcaactttacttcatgttgctgagatccctttcactgcttctgacccggaacccggatccggaagagggaacccacttgatgcccagaacaagcaaatgggtgaagggattaaggcgtttatgtctgcaagatctgacggatcaaaactacccacgtgtgttgttattgatgtcatgatgaactggagtaaagagatatttgttgattaccagattcctattgtctctttttttacttctggagctactaatactgctatgggttatggtaggtggaaagctaaaattggtgatctgaagcccggggagacccgtgtgatccccggacttcctactgaaatggccgttacttttgcggatttaaatcaaggtcctagaggccgtgggcctcggccggatgggtcaaggcctgacgggccaaggtctggaccacctggtgggatgaggtccggaccacctcacgggatgaggggtgggggacgaggtgggcggggcggtggacgacccggcccggatgcgaaaccacgttgggtagatgaagtggacgggtcggtagctttgcttatcaacacgtgtgacaatctcgagcgtgtgtttattgattacattgctgaagaaaccaagattcccgtttatggtgttggcccgttgctgcccgaaaagtattggaagtcagcgggttcgttgcttcgtgatcatgaaatgaggtctaaccataaagcgaattactcggaagatgaggtgtttcaatggctagaatcgaaaccagttgggtcggttatttacatatcgtttgggagtgaagttggcccgactatagacgagtataaagagttagctggatcattggaaggatcgaatcagaatttcatttgggtgatccagcccggttcggggataacgggcatgccaagatcgtttttgggcccggttaatacggatagtgaggaagaagaggaagggtattatcctgagggattagatgttaaagttgggaacaggggtttgatcatcactggatgggctccacagttgttgattttgagccacccatctacaggcgggttcttatcacattgtgggtggaattcaactgttgaggcgattgggcgaggtgttccgatattgggttggcccttgaggggtgatcagtttgataatgcgaaacttgtggcgaatcatttgaaaattgggtttgcgatgtcaagtgtggcgagtgaaggcggacgacctgggaagttcaacaaggagactataacagcagggattgagaaactaatgaatgatgaagatgtgcataaacaggcaaagaaacttagtaaagaatttgagagtgggtttccagtgagttcagttaaagcattgggtgctttcgtggagtctattagccagaaagcaacctaa

SEQ ID NO: 13 - DNA, 1378 bp, Helichrysum umbraculigerum
  source 1..1378: mol_type = other DNA
atgtcactcgtgactaataacccacatttactagtctacccattacctacctccggccatatcattccgttactcgacctgaccgaccttcttctccgccgtggcctcaccatcaccgtcgtgatatccaccacagaccttacgcttctcgacactctcctatcctcacaccccacgtctctacacaaactttacttccccgaccccgaaatcggcccatcttctcatcccgttattgccagaataattgccacccaaaaactatttgatccaattgttaaatggtttgaatcgcacccttcgcctccagtcgccatcatttccgacttctttcttgggtggactaatgaacttgcatcacgtttaggtattcgacgtgtggtgttttcaccttcgggagctcttggtcattccattttacaaagtttgtggcgtgacgtggcggagatcaatgcaaaaaatgttgatggaaatggaaactactcgatttcttttaccgatataccaaactcgcccgaatttcattggtggcagttgtcacaacttttgcgtgttcatagggagggagatccggacttcgaattttttaggaatggaatgttggctaatacgaaaagttggggtattgtttacaacacatttgaaaggattgaaaaggtttacattgaccatgtgaagaaacaaataggtcatgatcgggtatgggcaataggcccattacttcccgaagaacatggcccagttggtagcaccgcacgtggtgggtccagtgtagtgccacctcatgaccttctcacgtggttggacaaaaagccccatgactcggtcgtatatatatgttttgggagtcgattgacgttaagtgagaagcaaatgagtgcattagcaagtgcactcgagctcagtaacgttgattttattttgtgtgtgaaggcaagtggttcgagcttcattcctagtgggttcgaagatcgagtggtgggtcgggggttcgtaatcaaaggttgggccccacagttggcgatattgagacatcgggctgtggggtcgtttgtgactcattgtgggtggaactcaacattggaaggtgtttcatcaggagtgatgatgttgacgtggccaatgggtgcagaccaatatgcaaatgctaagctattggtcgaccagttaggtgttgggaaacgagtttgtgaaggtggacccgagagtgttcctgattcaactgagttggctcggttgttggaagagtcactgagtggtgatacatccgagcgagttaaagtcaaggagctaagtcgggaagctaacacagctgtgaaagaaggaacttcaataagagatttrgaacatgttcgttaaccttttatccgagctctaa

SEQ ID NO: 14 - protein, 466 aa, Helichrysum umbraculigerum
MTNSELVFIPSPGAGHLPPTVELAKLLLHREPQLSVTIIIMNLPHETKPTTETRMSTPRLRFIDIPKDESTKDLISRHTFISAFLEHQKPHVRNIVRSITESDSVRLVGFVVDMFCIAMMDVANELGAPTYLYFTSSAASLGLMFCLQAKRDDEEFDVTELKDKDSELSIPCYTNPLPAKLLPSVLFDKRGGSKTFIDLARKYRESRGIVVNTFQELESYAIEYLASSNANVPPVFPVGAILNQEKKVNDDKTEEIMTWLNEQPESSVVFLCFGSMGSFGEDQIKEIALAIEESGQRFLWSLRRPPSNENKYPKEYENFGEVLPEGFLERTSSVGKVIGWAPQMAVLSHSSVGGFVSHCGWNSTLESIWCGVPVAAWPLYAEQQLNAFKLVVELGLAVEIKIDYRSENEIILTSKEIESGIRRLMNDEELRMKVKEMKGNSRFAVSEGGSSYVSIRRFIDLVMTKE

SEQ ID NO: 15 - protein, 475 aa, Helichrysum umbraculigerum
MPTSELVFIPSPGVGHLSPTIELVNQLLHRDQRLSVTIIVMKFSLESKHDTETPTSTPRLRFIDIPYDESAMALINPNTFLSAFVEHNKPHVRNIVRDISESNSVRLAGFVVDMFCVAMTDVVNEFEIPTYIYFTSTANLLGLMFYLQAKRDDEGFDVTVLKDSESEFLSVPSYVNPVPAKVLPDAVLDKNGGSQMCLDLAKGFRESKGIIVNTFQELERRGIEHLLSSNMNLPPVFPVGPILNLRNAPNDGKTADIMTWLNDHPENSVVFLCFGSMGSFEKEQVKEIAIAIEQSGQRFLWSLRRPTSLEKFEFPKDYENPEEVLPKGFLERTKGVGKVIGWAPQMAVLSHPSVGGFVSHCGWNSTLESIWCGVPIAAWPLYAEQKINAFQLVVEMGMAAEIRIDYRTNTRPGGGKEMMVMAEEIESGIRKLMSDDEMRKKVKGMKDKSRAAVLEGGSSHTSIGILIENLVSITI

SEQ ID NO: 16 - protein, 285 aa, Helichrysum umbraculigerum
MVGLKCFWILQKGFRESKGIIVNTFQELERRGIEHLLSSNMDLPPVFPVGPILNLRNARNDGKMADIMTWLNDQPENSVVFLCFGSRGSFKEEQVKEIAIAIEQSGQRFLWSLRRPTSIETFEFPKYYENPEEVLPKGFLERTKSVGKVIGWAPQMAVLSHPSVGGFVSHCGWNSTLESIWCGVPIAAWPLYAEQQTNAFQLVVEMGMAAEIRIDYRTNTPLVGGKDMMVTAEEIERGIRKLMSDDEMRKKVKDMKDKSRGAVLEGGSSHTSIGNLIDVLVSITI

SEQ ID NO: 17 - protein, 488 aa, Helichrysum umbraculigerum
MATNNLHFLLIPHIGPGHTIPMIDMAKLLAKQPNVMVTIATTPLNITRYGHTLADAINSFRFFEVPFPAVEAGLPEGCESTDKIPSMDLVPNFLTAIGMLEQKLEEHFHLLEPRPNCIISDKYMSWTGDFADKYRIPRIMFDGMSCFNELCYNNLYENKVFEGMHETEPFVVPGLPDKIELTRKQLPPEFNPSSIDTSEFRQRARDAEVRAYGVVINSFEELEQEYVNEYKKLRKGKVWCIGPLSLCNSDNSDKAQRGNIASVDEEKCLKWLDSHEADSVVYACFGSLVRVNTPQLIELGLGLEASNRPFIWVVRSVHREKEVEEWLVESGFEERIKDRGLIIRGWAPQVLILSHPSIGGFLTHCGWNSTLESVCAGVPMITWPQFAEQFINEKLIVQVLGIGVGVGVDSVVHVGEEDRSGVKVKRESVTKAIEKVMDDEIDGNERRRRSKEFGKIANNAIKEGGSSYLNLTLLIQDIMRYANADASS

SEQ ID NO: 18 - protein, 468 aa, Helichrysum umbraculigerum
MEKTPHIAIVPSPGMGHLIPLVEFAKKLKNHHNIHATFIIPNDGPLSISQKVFLDSLPNGLNYLILPPVNFDDLPQDTQIETRISLMVTRSLDSLREVFKSLVVEKNMVALFIDLFGTDAFDVAIEFGVSPYVFFPSTAMALSLFLYLPKLDQMVSCEYRELPEPVQIPGCIPVRGQDLVDPVQDRKNDAYKWVLHNAKKYSMAKGIAVNSFKELEGGALNALLEDEPGKPKVYPVGPLVQTGFSCDVDSIECLKWLDGQPCGSVLYISFGSGGTLSSSQLNELAMGLELSEQRFIWVVRSPNDQPNATYFDSHGHKDPLGFLPKGFLERTKGIGFVIPSWAPQAQILSHSATGGFLTHCGWNSILETVVHGVPVIAWPLYAEQKMNAVSLTEGIKMALRPTVGENGIVGRLEVARVVKSLLEGEEGKAIRSRVRDLKDAAANVLSKDGSSTKTLDQLAVQLKKQELS

SEQ ID NO: 19 - protein, 180 aa, Helichrysum umbraculigerum
MTQKQMQMQPHFLLVTYPAQGHINPSLQFAERLIRLGVKVTFTTTVSAYRRMSKAGNISEFLNFAAFSDGFDDGFNFETDDHGLFLTQLRSRGKDSLKETILSNAKNGTPISCLVYTLLLPWAPEVARGLNVPSAFLWIQPASVLRLYYYYFNGYNELIGDDCNEPSWSIQLPGLPLLKS

SEQ ID NO: 20 - protein, 253 aa, Helichrysum umbraculigerum
MTKIQQQPHFLLVTYPAQGHINPSLRFAERLIRLGVKVTFTITVSAYRRMSKAGHISEFLNFAVFSDGFDDGFNSKTDDYGLFLTQFRSRGKDSLKETILSNAKNGTPVSCLVYTLLLPWAPEVARGLNVPSAFLWIQPASVLRLYYYYFNGYNELIGDDCNEPSWSIQLPGLPLLKSRDLPSFCLPSNPYADVLTLVKEHLDVLDLEEKPKILVNSFDELEREALNEIDGKLKMVAVGPLIPSAFFGWTGCI

SEQ ID NO: 21 - protein, 337 aa, Helichrysum umbraculigerum
MGSWRNSRTTSTKFLWLILPLMVVTVIIGVKKSNYGSKYNYPWVWSSVINSYSSSAVKEDVTVVAEGPVESFGLRSTVVNGGGVVAEGPSEDFGFNSSYPPLAMEDEMDVELPAIAKEDDLNATLSGPDLFVSANQTGGLHVDIGINSKYTSLDKLEARLGQVRAAIKEAESGNRTYDPDYVPEGPMYWHAASFHRSYLEMEKQFKVFVYEEGEPPIFHNGPCKNIYAMEGNFIYHMETTKFRTKNPEKAHTFFLPMSAAMMVRFIFERDPNVDHWRPMKQTIKDYVDLVGGKYPFWNRSLGADHFTVACHDWVSKVFYPIIFMLLLVFIFRMSTGC

SEQ ID NO: 22 - protein, 469 aa, Helichrysum umbraculigerum
MSTVEVAKLLVNRDHRLFITFLIIQPPSSGSGSAITTYIESLAEKAMDRISFIELPQDKIPPPRYPKSLPTAESKAHPLIFMIEFIKCHCKYVRNIVSDMISQPSSGRVAGLVIDMLCFSMMDVANEFNIPTYVFVTSNAAFLGFYLYVQILSNDQNQDVVELSKSDTEISVPGFVKPVPTKVFWTVVRTKEGLDFVLSSAQKLRQAKAIMVNTFLELETHAIKSLSDDTSIPPVYPVGPILNLEGGAGKTFDNDISRWLDSQPPSSVVFLCFGSHGCFDEIQVKEIAHALEQSGHRFLWSLRRPPSDQTLKVPGDYEDPGVVLPEGFLERTAGRGKVIGWAPQVMVLAHRAVGGFVSHCGWNSLLESLWFGVPTATWPIYAEQQMNAFEMVVELGLAVEITLDYRNDMDMFIVTAQEIESGIRKVMEDNEVRTKVKERSEKSRAAVAEGGSSYASVGHLIKEFTGNIS

SEQ ID NO: 23 - protein, 390 aa, Helichrysum umbraculigerum
MSSFINFVESTTQLQPQFEQLIQTLLPITAIISDGFLMWTQDSAEKFNIPRLVFYGTNIFFMTMCNIMAQFKPHAAVNSDDEAFDVPGFTRFKLTANDFEPPFNEVEPKGSMLDFLLEQQKAMVRSHGLVVNSFYEIEHEFNVYWNQNYGPKAWLMGPFCVAKPYASNVMDSEISTKVVKKSAWIQWLDRKLAANEPVLYISFGTQAEASMEHLHEVAIGLERSNVSFIWVVKAKQMQLIGAGFEERVKGRGKVVTEWVDQMEILKHEIVSGFLSHCGWNSLLESMCVGVPVLAMPLMADQLLNARLVVEEIGMGLRLWPRGMVARGIVGAEEVEKMVVELMEGEGGRRVRKRVIEVREMAYGAMKEGGSSSRTLDSLIDHVCEAFHKTV

SEQ ID NO: 24 - protein, 459 aa, Helichrysum umbraculigerum
MGSLKKGAHILIFPFPAQGHMLPLLDLTHHLATNGLTITILVTPKNLPILNPLLSSSPNIQPLVFPFPPHPRLPPHVENVKDIGNHANVPITNSLAKLQDQIIQWFNSHHNPPVAIISDFFLGWTQHLANKLGIPRVGFFSSGAYLTAVLDYVCHNIKTVRSQEETVFHDLPNSPCFKFEHLPGLAQIYKESDPEWELVLDGHIANGLSWGWIVNTFDGLESRYMEYLTKKMGVGRVFGVGPVNLLNGSDPMTRGKSESGSDSGVLNWLDGKPDGSVLYVCFGSQKFLTNDQMEGLSIGLEQSGVHYVWVVKDEQGDAIRSGSGRGLVVTGWAPQVSILGHGAVGGFLSHCGWNSVLEAIVNGVMILAWPMEADQFVNAKLLVDDHGIGVWVCEGPNTVPDSTELARKIGESMSTDKSEKVKAKEMKNKANEAVKEGGSSSMELSRLVKELSNFETNGP

SEQ ID NO: 25 - protein, 536 aa, Helichrysum umbraculigerum
MDTQTQVKKQKLETMEHKTSSAEIFVLPFFGTGHINPAMELCRNISSHNYKTTLIIPSHLSSSIPSPFSSTLLHVAEIPFTASDPEPGSGRGNPLDAQNKQMGEGIKAFMSARSDGSKLPTCVVIDVMMNWSKEIFVDYQIPIVSFFTSGATNTAMGYGRWKAKIGDLKPGETRVIPGLPTEMAVTFADLNQGPRGRGPRPDGSRPDGPRSGPPGGMRSGPPHGMRGGGRGGRGGGRPGPDAKPRWVDEVDGSVALLINTCDNLERVFIDYIAEETKIPVYGVGPLLPEKYWKSAGSLLRDHEMRSNHKANYSEDEVFQWLESKPVGSVIYISFGSEVGPTIDEYKELAGSLEGSNQNFIWVIQPGSGITGMPRSFLGPVNTDSEEEEEGYYPEGLDVKVGNRGLIITGWAPQLLILSHPSTGGFLSHCGWNSTVEAIGRGVPILGWPLRGDQFDNAKLVANHLKIGFAMSSVASEGGRPGKFNKETITAGIEKLMNDEDVHKQAKKLSKEFESGFPVSSVKALGAFVESISQKAT

SEQ ID NO: 26 - protein, 458 aa, Helichrysum umbraculigerum
MSLVTNNPHLLVYPLPTSGHIIPLLDLTDLLLRRGLTITVVISTTDLTLLDTLLSSHPTSLHKLYFPDPEIGPSSHPVIARIIATQKLFDPIVKWFESHPSPPVAIISDFFLGWTNELASRLGIRRVVFSPSGALGHSILQSLWRDVAEINAKNVDGNGNYSISFTDIPNSPEFHWWQLSQLLRVHREGDPDFEFFRNGMLANTKSWGIVYNTFERIEKVYIDHVKKQIGHDRVWAIGPLLPEEHGPVGSTARGGSSVVPPHDLLTWLDKKPHDSVVYICFGSRLTLSEKQMSALASALELSNVDFILCVKASGSSFIPSGFEDRVVGRGFVIKGWAPQLAILRHRAVGSFVTHCGWNSTLEGVSSGVMMLTWPMGADQYANAKLLVDQLGVGKRVCEGGPESVPDSTELARLLEESLSGDTSERVKVKELSREANTAVKEGTSIRDLNMFVNLLSEL

(19) United States
(12) Patent Application Publication
(10) Pub. No.: US 2025/0207145 A1
(43) Pub. Date: Jun. 26, 2025

(54) URIDINE DIPHOSPHATE-GLYCOSYLTRANSFERASE AND A TRANSGENIC CELL, TISSUE, AND ORGANISM COMPRISING SAME

(71) Applicant: YEDA RESEARCH AND DEVELOPMENT CO. LTD., Rehovot (IL)

(72) Inventors: Asaph AHARONI, Tel Aviv (IL); Paula BERMAN, Rehovot (IL); Luis DE-HARO, Rehovot (IL); Adam JOZWIAK, Rehovot (IL)

(21) Appl. No.: 18/851,662

(22) PCT Filed: Apr. 13, 2023
(86) PCT No.: PCT/IL2023/050392

§ 371 Date: Sep. 26, 2024

Related U.S. Application Data
(60) Provisional application No. 63/330,490, filed on Apr. 13, 2022.

(51) Int. Cl. C12N 15/82; C12N 9/10; C12P 19/46

(52) U.S. Cl. CPC C12N 15/8243; C12N 9/1051; C12P 19/46; C12Y 204/01017

(57) ABSTRACT

The present invention provides polynucleotide sequences derived from Helichrysum umbraculigerum and encoding a protein or a plurality thereof belonging to the uridine diphosphate (UDP)-glycosyltransferase (UGT) family. Further provided are an artificial nucleic acid molecule including the polynucleotide disclosed herein, a transgenic cell, tissue, or plant including same.

### CROSS-REFERENCE TO RELATED APPLICATIONS

This application claims the benefit of priority of U.S. Provisional Patent Application No. 63/330,490, titled “URIDINE DIPHOSPHATE-GLYCOSYLTRANSFERASE AND A TRANSGENIC CELL, TISSUE, AND ORGANISM COMPRISING SAME”, filed Apr. 13, 2022. The contents of which are all incorporated herein by reference in their entirety.

### REFERENCE TO AN ELECTRONIC SEQUENCE LISTING

The contents of the electronic sequence listing (YEDA-P-013-PCT ST26.xml; size: 45,465 bytes; and date of creation: Apr. 13, 2023) is herein incorporated by reference in its entirety.

### FIELD OF INVENTION

The present invention relates to uridine diphosphate (UDP)-glycosyltransferase (UGT) and a transgenic cell, tissue, and organism comprising same including polynucleotides encoding same, and methods of using same, such as for producing glycosylated cannabinoids or precursors thereof.

### BACKGROUND

Cannabinoids are typical of Cannabis sativa L. (Cannabis), although some specific compounds have also been identified in other flowering plants, liverworts, and fungi. One of these plants is Helichrysum umbraculigerum Less (Helichrysum). This perennial South-African plant is the only known plant other than Cannabis, producing cannabigerolic acid (CBGA), the five-carbon alkyl precursor of all the major cannabinoids.

In the past few years, the therapeutic usage of cannabinoids has made a significant leap as new reports highlight their potential for various medical purposes. However, one of the main challenges in cannabinoid pharmaceutical research and development is increasing their aqueous solubility and improving their oral bioavailability and absorption into the bloodstream. A possible strategy to overcome this challenge might be the glycosylation of cannabinoids. In plants, uridine diphosphate (UDP)-glycosyltransferases (UGTs) catalyze the covalent addition of sugars to a broad range of lipophilic molecules, increasing its solubility. Notably, endogenous cannabinoids glycosylation activity has been demonstrated in several plant species that do not synthesize cannabinoids naturally. Moreover, UGT genes from rice (Oryza sativa) and stevia (Stevia rebaudiana) were recently identified to glycosylate cannabinoids and reported in a research paper and patents.

Nonetheless, these enzymes were identified in plants that do not produce cannabinoids, therefore, their ability to glycosylated other substrates, e.g., not their natural substrates, such as cannabinoids, or their precursors, is unclear.

Therefore, there is a need for developing methodologies that allow large-scale glycosylation of cannabinoids or their precursors, which may increase their processability and/or applicability.

### SUMMARY

The present invention, in some embodiments, is based, in part, on the identification of glycosylated forms of cannabinoids and their precursor olivetolic acid (OA) in plants. According to sequence similarity to UGT enzymes from Arabidopsis thaliana, which were previously identified to glycosylate 2,4-dihydroxybenzoic acid (2,4-DHBA, a compound structurally similar to OA), the inventors discovered UGT genes/enzymes from Helichrysum that catalyze the glycosylation of OA and cannabinoids. The identified UGTs, which naturally glycosylate cannabinoids in planta, are likely to be more efficient on these compounds compared to the non-specific enzymes, currently suggested for use, such as from rice and stevia.

According to a first aspect, there is provided an isolated DNA molecule comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or any combination thereof.

According to another aspect, there is provided an artificial nucleic acid molecule comprising the isolated DNA molecule disclosed herein.

According to another aspect, there is provided a plasmid or an agrobacterium comprising the artificial nucleic acid molecule disclosed herein.

According to another aspect, there is provided an isolated protein encoded by any one of: (a) the isolated DNA molecule disclosed herein; (b) the artificial vector disclosed herein; and (c) the plasmid or agrobacterium disclosed herein.

According to another aspect, there is provided a transgenic cell comprising: (a) the isolated DNA molecule disclosed herein; (b) the artificial nucleic acid molecule disclosed herein; (c) the plasmid or agrobacterium disclosed herein; (d) the isolated protein disclosed herein; or (e) any combination of (a) to (d).

According to another aspect, there is provided an extract derived from the transgenic cell disclosed herein, or any fraction thereof.

According to another aspect, there is provided a transgenic plant, a transgenic plant tissue or a plant part, comprising: (a) the isolated DNA molecule disclosed herein; (b) the artificial vector disclosed herein; (c) the plasmid or agrobacterium disclosed herein; (d) the isolated protein disclosed herein; (e) the transgenic cell disclosed herein; or (f) any combination of (a) to (e).

According to another aspect, there is provided a composition comprising: (a) the isolated DNA molecule disclosed herein; (b) the artificial vector disclosed herein; (c) the plasmid or agrobacterium disclosed herein; (d) the isolated protein disclosed herein; (e) the transgenic cell disclosed herein; (f) the extract disclosed herein; (g) the transgenic plant tissue or plant part disclosed herein; or (h) any combination of (a) to (g), and an acceptable carrier.

According to another aspect, there is provided a method for glycosylating a cannabinoid comprising or a precursor thereof: (a) providing a cell comprising an artificial vector comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13; and (b) culturing the cell from step (a) such that a protein encoded by the artificial vector is expressed, thereby glycosylating a cannabinoid comprising or a precursor thereof.

According to another aspect, there is provided an extract of a cell obtained according to the method disclosed herein.

According to another aspect, there is provided a medium or a portion thereof separated from a cultured cell, obtained according to the method disclosed herein.

According to another aspect, there is provided a composition comprising: (a) the extract disclosed herein; (b) the medium or a portion thereof disclosed herein; or (c) a combination of (a) and (b), and an acceptable carrier.

According to another aspect, there is provided a method for glycosylating a cannabinoid or a precursor thereof, the method comprising contacting the cannabinoid or precursor thereof with an effective amount of a protein comprising an amino acid sequence with at least 90% homology to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, thereby glycosylating the cannabinoid or a precursor thereof.

In some embodiments, the nucleic acid sequence has at least 87% homology to any one of SEQ ID Nos.: 1-13 is 700 to 1,800 nucleotides long.

In some embodiments, the nucleic acid sequence encodes a protein being a uridine 5′-diphospho (UDP)-glucuronosyltransferase (UGT).

In some embodiments, the isolated protein comprises an amino acid sequence with at least 90% homology to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

In some embodiments, the isolated protein consists of an amino acid sequence of SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26.

In some embodiments, the isolated protein is characterized by being capable of glycosylating a cannabinoid or a precursor thereof.

In some embodiments, the transgenic cell is any one of: a unicellular organism, a cell of a multicellular organism, and a cell in a culture.

In some embodiments, the unicellular organism comprises a fungus or a bacterium.

In some embodiments, the fungus is a yeast cell.

In some embodiments, the extract comprises the isolated DNA molecule, the isolated protein, or both.

In some embodiments, the transgenic plant is a Cannabis sativa plant.

In some embodiments, the cell is a transgenic cell, or a cell transfected with the isolated DNA molecule disclosed herein or the artificial vector disclosed herein.

In some embodiments, the protein is characterized by being capable of transferring a glucuronic acid component of UDP-glucuronic acid to the cannabinoid or precursor thereof.

In some embodiments, the culturing comprises supplementing the cell with an effective amount of UDP.

In some embodiments, the artificial vector is an expression vector.

In some embodiments, the cell is a prokaryote cell or a eukaryote cell.

In some embodiments, the method further comprises a step (c) comprising extracting the cell, thereby obtaining an extract of the cell.

In some embodiments, the method further comprises a step preceding step (c), comprising separating the cultured cell from a medium wherein the cell is cultured.

In some embodiments, the method further comprises a step preceding step (a), comprising introducing or transfecting the cell with the artificial vector.

In some embodiments, the contacting is in a cell-free system.

In some embodiments, the cannabinoid is CBDA, CBGA, heliCBGA, or any combination thereof.

In some embodiments, the cannabinoid precursor is olivetolic acid (OA).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1C include graphs showing the identification of compounds in a Helichrysum ethanolic extract. Extracted ion current (XIC) chromatograms and MS/MS spectral matching of (1A) cannabigerolic acid (CBGA, 359.222 Da), (1B) heli cannabigerolic acid (heliCBGA, 393.206 Da), and (1C) olivetolic acid (OA, 223.097 Da) standards or authentic compounds versus a Helichrysum sample.

FIGS. 2A-2E include a table and chemical structure elucidation of CBGA and heliCBGA via 1D and 2D NMR. (2A) ¹H and ¹³C chemical shift assignment, (2B-2C) atom numbering and COSY correlations, and (2D-2E) HMBC correlations of CBGA and heliCBGA, respectively. The carbon on the carboxyl of HeliCBGA was not observed in the NMR spectra, however, the LC/HRMSMS spectra and chemical formula confirm the presence of this group.

FIGS. 3A-3E include a table and chemical structure elucidation of Glc-OA and Glc-DHSA via 1D and 2D NMR. (3A) ¹H and ¹³C chemical shift assignment, (3B-3C) atom numbering and COSY correlations, and (3D-3E) HMBC correlations of Glc-DHSA and Glc-DHSA, respectively. The carbon on the carboxyl was not observed in the NMR spectra. However, LC/HRMSMS spectra and chemical formula confirm the presence of this group.

FIGS. 4A-4G include graphs and chemical structure elucidation showing the identification of glucosylated intermediates, cannabinoids and amorfrutins in a Helichrysum ethanolic extract. (4A) Comparison of MS/MS spectra in negative polarity of Glc-OA and Glc-DHSA versus OA and DHSA, respectively. As shown, the glucosylated compounds exhibited neutral losses of 162.053 Da corresponding to the loss of a hexose and similar fragments as the non-glucosylated compounds. The differences in the relative abundances of the fragment ions are probably due to the large difference in masses between the glucosylated and the non-glucosylated compounds. Extracted ion current (XIC) chromatograms (4B) of m/z 357.119, 371.136, 385.150 and 399.166 and MS/MS spectra in negative polarity of (4C) unlabeled and isotopically labeled (4D) glucosylated alkyl intermediates. The marked peaks in each chromatogram correspond with the detected glucosylated intermediates. As shown, the alkyl homologues elute from the reversed phase column in order of chain length as a result of increasing lipophilicities. For all alkyl homologues, an appropriate m/z shift in the MS/MS spectra of all the product ions that include the alkyl chain was observed. Isomers with similar masses and MS/MS fragmentations were observed for Glc-OA and Glc-HA which the inventors assigned as branched short-chain FAs according to feeding experiments, and in agreement with the identified cannabinoids. (4E) Suggested fragmentation structure of Glc-OA according to MS/MS spectra and labeling. Fragments colored in blue correspond to the m/z of the specific fragment in the compound labeled with hexanoicD₁₁ acid. The fragments are similar to those observed for the non-glucosylated compounds. (4F) MS/MS spectra in negative polarity of Glc-CBPA, Glc-CBGA, Glc-heliCBPA and Glc-heliCBGA. Identification was by similar MS/MS fragmentations as the non-glucosylated compounds and relative RT. (4G) Structures of the observed compounds and isotopically labeled precursors of the identified compounds. IP, isoprenyl; MP, monoprenyl.

FIGS. 5A-5G include a graph and micrographs showing CBGA and Glc-OA content in plant tissues and localization of CBGA to glandular trichomes of Helichrysum leaves and flowers. (5A) CBGA absolute concentrations (% w/w, n=4), and peak areas of Glc-OA (according to UPLC-qTOF peak areas of m/z of 385.15) in different Helichrysum plant tissues. Optical images (5B and 5D) and MALDI-MSI (5C and 5E) of m/z 361.24±0.01 Da (corresponding with the protonated mass of CBGA) of a cross sectioned leaf and a flower receptacle. Trichomes in 5B and 5D are marked to improve interpretation. CBGA is localized to stalked glandular trichomes. Optical images of the (5F) flower head and (5G) cut receptacle for reference. Stalked glandular (yellow arrow) and mechanical trichomes (white arrow) can be observed on the surface border of the tissue in (5G). The white broken lines in (5C) and (5E) mark the regions analyzed. Scale bar: 100 μm (5B); 500 μm (5C); 200 μm (5D); 1,000 μm (5E); 1,000 μm (5F); and 500 μm (5G).

FIGS. 6A-6C include graphs showing expression profiling of Helichrysum genes (UMI-aware 3′ Trans-seq). (6A) PCA plot of the normalized gene expression distribution of the 21 samples sequenced. (6B) Over-representation analysis (ORA) of groups of genes belonging to each coexpression module obtained with CEMI-tools. Module number M4 includes genes enriched in trichomes and in leaves (6C) Normalized expression of the genes belonging to the module number 4.

FIG. 7 includes a graph showing expression profiles of selected UGT Helichrysum genes (UMI-aware 3′ Trans-seq). CPM normalized expression of selected UGT genes with expression patterns correlated with CBGA accumulation. A secondary axis including CBGA quantification is included in the right side of the plot.

FIGS. 8A-8C include chemical structures and graphs showing activities of lysates containing HuUGTs with (8A) OA, (8B) CBGA and (8C) heliCBGA as substrates and UDP-glc as the sugar donor. Reactions show differing substrate specificities and type of products produced. Peaks are according to the annotation on the chromatograms for HuUGT1. The most abundant products are marked with asterisks. EV, empty vector.

FIG. 9 includes spectra showing functional characterization of UGTs. Extracted ion chromatograms of the observed monoglucosides according to the theoretical m/z values, following enzymatic assays with the purified enzymes (HuUGT1, HuUGT6, HuUGT11, HuUGT13, OsUGT and SrUGT) in the presence of UDP and either OA, DHSA, CBGA, heliCBGA, CBDA, Δ⁹-THCA, CBCA, olivetol, CBG, CBD or Δ⁹-THC. One to three glucosylated compounds were observed for each substrate according to the possible cites of glucosylation marked on each structure. The monoglucosides were identified according to MS/MS fragmentation and assigned by fragmentation patterns (FIG. 10).

FIG. 10 includes MS/MS spectra of observed glucosylated compounds following in vitro assays with UGTs from Helichrysum, stevia, and rice. Assignment of peaks (1-3) was according to MS/MS fragmentation patterns and the m/z difference between the parent and fragment1. The retention times of peaks 1 and 2 were constant, whereas peak 3 eluted at different relative RTs. The XIC chromatograms for each substrate following a reaction with SrUGT or HuUGT6 are shown as reference.

FIG. 11 includes LC/MS chromatograms of the observed diglucosides following enzymatic assays with the purified enzymes in the presence of UDP-glc and the cannabinoid acceptors. All LC/MS chromatograms were selected for the theoretical m/z values of the respective compounds of interest.

FIGS. 12A-12B include curves and a table showing comparison of steady state kinetic analysis of HuUGT11 and HuUGT13 versus OsUGT and SrUGT, with olivetolic acid and UDP-glc. (12A) The Michaelis-Menten Km value of each enzyme was calculated using varying (0.5 μM-3 mM) and constant (1 mM) concentrations of olivetolic acid and GPP (n=3 technically independent samples; measurements were plotted individually). Since there was no analytical standard available for Glc-OA, V₀ was calculated using the calibration curve of OA. (12B) A summary of the results presented in 12A. V₀ and Vₘₐₓ were calculated using the calibration curve of OA since there was no analytical standard available for Glc-OA.

### DETAILED DESCRIPTION

The present invention, in some embodiments, is directed to polynucleotide sequences derived from Helichrysum umbraculigerum and encoding a protein or a plurality thereof belonging to the uridine diphosphate (UDP)-glycosyltransferase (UGT) family.

According to some embodiments, there is provided a polynucleotide comprising a nucleic acid sequence comprising any one of SEQ ID Nos.: 1-13, or any combination thereof.

In some embodiments, the polynucleotide is an isolated polynucleotide. In some embodiments, the polynucleotide is a DNA molecule. In some embodiments, the polynucleotide is an isolated DNA molecule. In some embodiments, the DNA molecule is an isolated DNA molecule. In some embodiments, the DNA molecule is a complementary DNA (cDNA) molecule.

As used herein, the terms “isolated polynucleotide” and “isolated DNA molecule” refers to a nucleic acid molecule that is essentially free from contaminating cellular components, such as carbohydrate, lipid, or other proteinaceous impurities associated with the nucleic acid in nature. Typically, a preparation of isolated DNA or RNA contains the nucleic acid in a highly purified form, e.g., at least about 80% pure, at least about 90% pure, at least about 95% pure, greater than 95% pure, or greater than 99% pure. In some embodiments, the isolated polynucleotide is any one of DNA, RNA, and cDNA. In some embodiments, the isolated polynucleotide is a synthesized polynucleotide. Synthesis of polynucleotides is well known in the art and may be performed, for example, by ligating or covalently linking by primer linkers multiple nucleic acid molecules together.

The term “nucleic acid” is well known in the art. A “nucleic acid” as used herein will generally refer to any molecule (e.g., a strand) of DNA, RNA or a derivative or analog thereof, comprising nucleotides. Nucleotides are comprised of nucleosides and phosphate groups. The nitrogenous bases of nucleosides include, for example, naturally occurring purine or pyrimidine nucleosides as found in DNA (e.g., an adenine “A,” a guanine “G,” a thymine “T” or a cytosine “C”) or RNA (e.g., an A, a G, an uracil “U” or a C).

The term “nucleic acid molecule” includes but is not limited to single-stranded RNA (ssRNA), double-stranded RNA (dsRNA), single-stranded DNA (ssDNA), double-stranded DNA (dsDNA), small RNAs, circular nucleic acids, fragments of genomic DNA or RNA, degraded nucleic acids, amplification products, modified nucleic acids, plasmid or organellar nucleic acids, and artificial nucleic acids such as oligonucleotides.

In some embodiments, the polynucleotide comprises or consists of the nucleic acid sequence:

(SEQ ID NO: 1)
ATGACCAACTCGGAACTTGTTTTCATCCCATCTCCGGGAGCCGGCCACCT
ACCACCTACGGTGGAGCTAGCAAAGCTCCTCCTCCACCGCGAACCACAGC
TTTCGGTTACCATCATCATCATGAACCTCCCTCATGAAACAAAACCCACT
ACTGAAACTCGAATGTCCACTCCTCGTCTACGCTTTATTGACATACCTAA
AGACGAGTCAACAAAAGATCTTATCTCACGCCACACATTCATATCCGCCT
TCCTTGAACACCAAAAGCCACATGTTCGAAACATTGTCCGTTCAATCACC
GAGTCTGACTCGGTTCGGTTAGTTGGGTTCGTCGTAGACATGTTTTGTAT
TGCCATGATGGACGTCGCAAACGAGCTGGGTGCTCCAACTTATCTTTATT
TCACCTCCTCTGCCGCTTCACTTGGCCTCATGTTTTGCCTACAGGCCAAA
CGAGACGACGAGGAGTTTGATGTGACCGAGTTGAAGGACAAAGATTCGGA
ACTCTCCATTCCGTGTTACACCAACCCACTCCCAGCTAAGTTGTTACCTT
CGGTACTATTTGATAAGAGAGGTGGGTCAAAAACATTTATTGACCTCGCT
AGAAAGTATCGCGAGTCGAGGGGTATAGTTGTAAATACTTTTCAAGAACT
CGAAAGCTATGCTATTGAGTATCTTGCAAGTAGTAATGCTAACGTCCCAC
CGGTGTTTCCGGTGGGGGCGATACTAAACCAAGAAAAAAAGGTAAATGAT
GATAAGACGGAGGAGATTATGACATGGTTAAACGAGCAACCGGAGAGTTC
GGTGGTGTTTCTATGCTTCGGGAGCATGGGAAGCTTCGGTGAGGATCAAA
TTAAGGAAATAGCGCTTGCTATCGAAGAAAGCGGACAAAGGTTTTTGTGG
TCACTACGTCGTCCCCCTTCGAACGAAAATAAGTACCCGAAAGAATACGA
AAATTTTGGAGAGGTTCTTCCGGAAGGTTTCCTTGAACGAACATCGAGTG
TAGGGAAAGTGATAGGATGGGCCCCACAAATGGCAGTGTTGTCCCATTCT
TCAGTTGGTGGGTTTGTGTCACATTGCGGATGGAACTCGACACTCGAGAG
CATATGGTGTGGTGTACCGGTAGCTGCGTGGCCATTATATGCAGAACAAC
AACTTAATGCTTTTAAACTAGTGGTGGAGTTGGGCTTAGCGGTCGAGATT
AAGATTGATTATAGGAGTGAGAACGAGATTATTTTGACATCGAAAGAAAT
CGAGAGTGGGATTAGGAGGTTGATGAATGATGAAGAGTTGAGGATGAAAG
TGAAAGAGATGAAGGGGAATAGTAGGTTTGCAGTTTCAGAGGGTGGATCT
TCTTACGTATCCATTAGGCGTTTTATCGACCTTGTGATGACTAAGGAGTA
A.

In some embodiments, the polynucleotide comprises a nucleic acid sequence with at least 77%, at least 79%, at least 85%, at least 95%, or at least 99% homology or identity to SEQ ID NO: 1, or any value and range therebetween. Each possibility represents a separate embodiment of the invention. In some embodiments, the polynucleotide comprises a nucleic acid sequence with 77% to 95%, 78% to 100%, 79% to 99%, or 77% to 100% homology or identity to SEQ ID NO: 1. Each possibility represents a separate embodiment of the invention.

In some embodiments, the polynucleotide comprises or consists of the nucleic acid sequence:

(SEQ ID NO: 2)
ATGCCGACCTCAGAACTTGTTTTCATCCCATCCCCCGGTGTCGGCCACCTGTCG
CCTACCATCGAACTCGTCAATCAACTCCTCCACCGCGACCAGCGCCTGTCTGTC
ACAATCATCGTCATGAAGTTCTCTCTTGAATCAAAACACGATACAGAAACTCC
TACATCCACTCCTCGATTACGCTTCATTGATATCCCTTATGACGAGTCCGCTAT
GGCTCTCATTAACCCGAACACGTTCCTCTCCGCTTTCGTCGAGCACAACAAACC
TCATGTTCGAAACATTGTTCGTGACATTTCCGAGTCTAACTCGGTTCGGCTCGC
GGGGTTTGTTGTGGACATGTTTTGTGTAGCTATGACGGATGTAGTGAACGAGTT
TGAAATTCCAACCTATATTTATTTTACCTCGACCGCGAACTTACTCGGACTCAT
GTTTTACCTTCAGGCCAAGCGTGACGACGAGGGTTTTGATGTCACCGTGTTGAA
AGACTCAGAATCAGAGTTTTTGTCTGTTCCGAGTTATGTCAACCCGGTTCCAGC
TAAGGTTTTACCTGATGCAGTTTTGGATAAGAATGGTGGGTCTCAAATGTGTCT
GGATCTTGCAAAAGGGTTTCGTGAGTCGAAGGGCATAATAGTAAATACATTTC
AAGAACTCGAAAGGCGTGGAATCGAGCACCTTTTAAGTAGTAACATGAACCTC
CCACCTGTGTTTCCTGTGGGGCCTATATTGAACTTGAGAAATGCGCCAAACGAT
GGTAAAACGGCCGATATCATGACATGGTTAAATGACCATCCAGAGAACTCGGT
TGTGTTCTTGTGTTTCGGAAGTATGGGAAGCTTCGAGAAAGAACAAGTGAAGG
AGATAGCGATTGCCATCGAACAGAGTGGGCAACGGTTTCTATGGTCACTCCGT
CGTCCAACATCGCTAGAAAAGTTTGAGTTTCCAAAGGATTACGAGAACCCGGA
GGAGGTTTTGCCAAAGGGATTTCTTGAAAGGACAAAAGGTGTGGGAAAGGTTA
TCGGGTGGGCCCCACAAATGGCGGTGTTGTCTCACCCGTCAGTGGGAGGGTTC
GTGTCCCACTGTGGGTGGAACTCCACATTGGAGAGCATATGGTGTGGGGTCCC
AATAGCGGCTTGGCCACTATATGCGGAACAAAAAATTAATGCTTTTCAATTGG
TGGTAGAGATGGGAATGGCAGCTGAGATTAGGATCGACTATCGGACTAATACG
AGACCGGGTGGTGGTAAAGAGATGATGGTAATGGCTGAAGAGATTGAGAGTG
GTATTAGGAAGTTGATGAGCGATGATGAGATGAGAAAGAAAGTGAAAGGTAT
GAAGGATAAAAGTAGGGCTGCTGTTCTTGAAGGTGGATCATCTCACACATCAA
TTGGGATTTTAATTGAGAATTTGGTGAGTATAACGATCTAG.

In some embodiments, the polynucleotide comprises a nucleic acid sequence with at least 76%, at least 77%, at least 85%, at least 93%, at least 97%, or at least 99% homology or identity to SEQ ID NO: 2, or any value and range therebetween. Each possibility represents a separate embodiment of the invention. In some embodiments, the polynucleotide comprises a nucleic acid sequence with 76% to 95%, 77% to 98%, 80% to 99%, or 76% to 100% homology or identity to SEQ ID NO: 2. Each possibility represents a separate embodiment of the invention.

In some embodiments, the polynucleotide comprises or consists of the nucleic acid sequence:

(SEQ ID NO: 3)
ATGGTGGGTCTCAAATGTTTTTGGATCTTGCAAAAAGGTTTTCGTGAGTCGAAG
GGCATAATAGTAAATACATTTCAAGAACTCGAAAGGCGTGGAATCGAGCACCT
TTTAAGTAGTAACATGGACCTCCCACCTGTGTTTCCTGTGGGGCCGATATTGAA
CTTGAGAAATGCGCGAAACGATGGTAAAATGGCCGATATCATGACATGGTTAA
ATGACCAGCCAGAGAACTCGGTTGTGTTCTTGTGTTTCGGAAGTAGGGGAAGC
TTCAAGGAGGAACAAGTGAAGGAGATAGCAATTGCCATCGAACAAAGTGGGC
AACGGTTTCTATGGTCACTCCGTCGTCCAACATCGATAGAAACGTTTGAGTTTC
CAAAGTATTACGAGAACCCGGAGGAGGTTTTGCCAAAGGGATTTCTTGAAAGG
ACAAAAAGTGTGGGAAAGGTTATCGGGTGGGCCCCACAAATGGCGGTATTGTC
TCACCCGTCAGTGGGAGGGTTCGTGTCCCACTGTGGGTGGAACTCCACATTGG
AGAGCATATGGTGTGGGGTCCCAATAGCGGCTTGGCCACTATATGCGGAACAA
CAAACTAATGCTTTTCAATTGGTGGTCGAGATGGGAATGGCAGCAGAGATTAG
GATCGACTATCGGACTAATACACCACTGGTTGGTGGTAAAGACATGATGGTAA
CGGCTGAAGAGATTGAGAGAGGTATTAGGAAGTTGATGAGCGATGATGAGAT
GCGAAAGAAAGTGAAAGACATGAAGGATAAGAGTAGAGGTGCAGTTTTAGAG
GGTGGGTCATCTCATACATCAATTGGGAATTTAATTGATGTTTTGGTGAGTATA
ACGATCTAG.

In some embodiments, the polynucleotide comprises a nucleic acid sequence with at least 78%, at least 80%, at least 85%, at least 95%, or at least 99% homology or identity to SEQ ID NO: 3, or any value and range therebetween. Each possibility represents a separate embodiment of the invention. In some embodiments, the polynucleotide comprises a nucleic acid sequence with 79% to 95%, 78% to 100%, 80% to 99%, or 79% to 100% homology or identity to SEQ ID NO: 3. Each possibility represents a separate embodiment of the invention.

In some embodiments, the polynucleotide comprises or consists of the nucleic acid sequence:

(SEQ ID NO: 4)
ATGGCGACCAACAACCTCCATTTCCTTCTAATTCCCCATATAGGTCCAGGCCAC
ACTATTCCCATGATAGATATGGCTAAACTTCTTGCAAAACAACCAAATGTAAT
GGTTACAATAGCTACAACACCTCTTAATATCACCCGTTACGGGCACACTCTCGC
AGACGCCATCAACTCGTTTCGCTTCTTTGAGGTTCCATTTCCGGCAGTTGAGGC
TGGATTACCTGAAGGATGTGAAAGCACGGATAAAATCCCAAGTATGGATCTAG
TACCGAACTTTTTAACCGCGATTGGTATGCTAGAACAAAAGCTAGAAGAGCAT
TTTCACTTGCTAGAGCCTCGTCCGAATTGTATTATTTCTGATAAGTACATGTCG
TGGACGGGTGATTTTGCTGATAAGTATCGGATCCCTAGAATTATGTTTGATGGA
ATGAGCTGTTTTAACGAGTTATGTTACAACAATTTGTATGAAAACAAGGTGTTT
GAAGGGATGCATGAAACAGAACCATTTGTTGTCCCTGGTTTACCCGATAAAAT
TGAGCTAACACGAAAACAGCTCCCACCTGAGTTTAACCCGAGCTCGATTGATA
CAAGTGAGTTTCGTCAGCGGGCTAGGGACGCTGAGGTGAGGGCTTATGGAGTT
GTGATCAATAGTTTTGAGGAGTTGGAACAAGAATATGTTAATGAGTATAAGAA
GTTAAGAAAGGGTAAGGTTTGGTGTATCGGCCCGCTGTCACTGTGCAATAGTG
ACAATTCGGATAAAGCCCAAAGAGGAAATATAGCGTCAGTCGATGAAGAAAA
ATGTTTAAAATGGCTTGATTCTCATGAAGCCGACTCAGTAGTTTACGCTTGTTT
TGGTAGCCTTGTTCGGGTCAACACCCCACAACTAATTGAGCTTGGTTTAGGCCT
AGAAGCATCAAATCGCCCGTTCATTTGGGTGGTTAGATCGGTTCATAGAGAAA
AAGAGGTCGAGGAATGGCTAGTGGAAAGTGGTTTTGAGGAGAGAATTAAAGA
TAGAGGTTTAATAATCCGAGGTTGGGCCCCACAAGTACTTATCTTGTCTCACCC
TTCTATTGGAGGGTTTTTAACGCATTGCGGTTGGAACTCGACCCTAGAATCAGT
CTGTGCAGGTGTTCCAATGATCACATGGCCTCAATTTGCAGAGCAATTTATCAA
CGAGAAGCTAATAGTGCAAGTGTTGGGGATTGGTGTGGGTGTTGGAGTTGATT
CTGTTGTCCATGTGGGCGAAGAAGATAGATCTGGGGTGAAAGTGAAGAGGGA
GAGTGTTACGAAGGCTATTGAGAAAGTCATGGATGACGAGATTGATGGAAATG
AGAGACGGAGGAGATCGAAAGAGTTTGGAAAGATAGCTAATAACGCGATTAA
AGAGGGAGGGTCTTCATACCTTAACTTGACTCTGCTAATTCAGGACATAATGC
GTTATGCAAATGCAGATGCTTCAAGCTAA.

In some embodiments, the polynucleotide comprises a nucleic acid sequence with at least 87%, at least 92%, at least 97%, or at least 99% homology or identity to SEQ ID NO: 4, or any value and range therebetween. Each possibility represents a separate embodiment of the invention. In some embodiments, the polynucleotide comprises a nucleic acid sequence with 87% to 100%, 88% to 99%, 89% to 99%, or 87% to 100% homology or identity to SEQ ID NO: 4. Each possibility represents a separate embodiment of the invention.

In some embodiments, the polynucleotide comprises or consists of the nucleic acid sequence:

(SEQ ID NO: 5)
ATGGAAAAAACACCTCATATAGCCATTGTACCAAGTCCAGGAATGGGCCACTT
GATCCCTTTAGTTGAGTTTGCTAAAAAACTAAAAAATCACCACAACATACATG
CAACTTTCATCATCCCAAATGATGGACCTTTATCTATTTCTCAAAAGGTTTTTCT
TGATTCACTTCCTAATGGTTTAAACTATCTCATTCTACCTCCGGTAAATTTTGAT
GATTTACCACAAGATACCCAAATCGAAACTCGAATTAGTCTAATGGTAACACG
GTCTCTTGATTCGCTACGTGAAGTGTTTAAGTCATTAGTTGTGGAAAAAAATAT
GGTTGCTTTGTTTATTGATCTTTTTGGGACAGATGCATTTGATGTTGCTATTGAA
TTTGGTGTTTCACCTTATGTGTTCTTTCCATCAACTGCTATGGCTTTATCTTTGTT
TCTATATTTGCCTAAACTTGATCAGATGGTTTCATGTGAGTATAGGGAGCTTCC
TGAACCGGTTCAAATTCCAGGTTGTATACCGGTTCGTGGACAAGACTTGGTTG
ACCCGGTTCAAGATAGAAAGAATGATGCATACAAATGGGTGCTTCATAATGCA
AAGAAGTATTCAATGGCTAAGGGTATAGCGGTAAATAGCTTCAAGGAGTTAGA
AGGTGGAGCTTTGAATGCTTTGCTAGAAGATGAACCGGGTAAGCCAAAAGTTT
ATCCGGTCGGACCGTTAGTACAAACCGGTTTTAGTTGTGATGTTGATTCGATAG
AGTGCTTGAAGTGGTTAGATGGTCAGCCATGTGGTTCTGTTTTGTATATATCTT
TTGGAAGCGGTGGGACCCTTTCATCCAGTCAACTTAATGAGTTAGCTATGGGTT
TGGAGTTGAGTGAACAACGGTTCATATGGGTGGTTAGAAGCCCGAACGATCAA
CCAAACGCCACGTACTTTGATTCTCATGGTCACAAAGACCCTCTTGGTTTTTTG
CCCAAAGGGTTCTTGGAAAGAACCAAAGGAATTGGGTTTGTGATCCCTTCTTG
GGCTCCACAAGCCCAGATCCTGAGTCACAGTGCCACAGGTGGATTTTTAACCC
ACTGTGGTTGGAACTCAATTCTCGAGACTGTAGTCCATGGTGTGCCGGTGATTG
CTTGGCCACTTTATGCCGAGCAAAAGATGAATGCAGTGTCTTTAACCGAGGGT
ATAAAAATGGCGTTAAGACCCACGGTTGGTGAAAATGGGATTGTGGGTCGCTT
AGAGGTTGCGAGAGTTGTGAAGAGTTTACTGGAAGGAGAAGAAGGGAAGGCG
ATTAGGAGTCGAGTTCGTGATCTCAAGGATGCTGCTGCTAATGTTCTTAGTAAA
GATGGGTCTTCTACAAAAACTTTAGATCAATTGGCTGTACAGTTGAAAAAACA
AGAATTAAGCTAG.

In some embodiments, the polynucleotide comprises a nucleic acid sequence with at least 87%, at least 92%, at least 97%, or at least 99% homology or identity to SEQ ID NO: 5, or any value and range therebetween. Each possibility represents a separate embodiment of the invention. In some embodiments, the polynucleotide comprises a nucleic acid sequence with 87% to 100%, 88% to 99%, 89% to 99%, or 87% to 100% homology or identity to SEQ ID NO: 5. Each possibility represents a separate embodiment of the invention.

In some embodiments, the polynucleotide comprises or consists of the nucleic acid sequence:

(SEQ ID NO: 6)
ATGACTCAAAAGCAAATGCAAATGCAACCTCACTTTCTCTTAGTAACATATCCC
GCACAAGGTCATATTAACCCGTCTCTCCAGTTCGCTGAACGTCTCATTCGGTTG
GGTGTCAAAGTCACCTTCACAACAACTGTCTCTGCTTACCGCCGAATGAGTAA
AGCGGGCAACATCTCAGAGTTTTTAAATTTTGCTGCTTTTTCAGACGGCTTTGA
TGACGGTTTCAACTTCGAAACAGACGATCATGGTCTCTTCTTAACTCAATTGAG
AAGCAGGGGAAAAGATAGCTTGAAAGAAACAATTCTTTCAAATGCTAAAAAT
GGAACTCCAATTAGTTGTTTGGTTTACACACTCCTACTCCCTTGGGCTCCTGAG
GTGGCACGTGGCCTAAACGTGCCCTCAGCCTTTCTTTGGATTCAACCAGCTTCT
GTTTTACGACTTTACTATTACTACTTCAATGGGTACAATGAACTCATCGGTGAC
GATTGTAATGAACCTTCATGGTCCATTCAATTACCAGGGTTACCATTGCTCAAA
AGTCATGACCTTCCCTCCTTTTGTCTCCCTTCAAATCCTTACAGTAATGTACTGG
CTCTAGTCAAAGAGCATTTAGATATGCTGGATCTGGAAGAGAAGCCTAAAATA
CTTGTGAATAGTTTTGATGAGTTGGAGAGGGAGGCGTTGAATGAAATTAATGG
AAAACTAAAAATGGTCGCCGTAGGGCCTTTGATTCCATCAGCTTTTTTGGATGG
ACAAGATGCATCTGACAAATCTTTTAGGGGAGATTTGTTTGAAACATCCAAAG
ATTATTTGGAATGGATGAATACAAAGCCTGAAGGGTCCATTGTTTACATATCTT
TTGGTAGTCTTTTAGTGTTCTCAAAGATACAAAAGGAGGCAATGGCACATGCT
TTGTTAGAGTGCGGGAGGCCGTTCTTGTGGGTGATAAGAGATGGAGAACAAGG
AGAACAACTAAGTTGTATTGAGAAATTGGAACAATTAGGTTTGATAGTCCCAT
GGTGTAGTCAACTAGAGGTATTATCACACCCTTCTTTAGGTTGTTTTGTGACAC
ATTGTGGTTGGAACTCGACTTTAGAGAGTATAGTTTGTGGAGTTCCTGTGGTGG
CATTTCCTCAATGGACAGATCAGACGACAAATGCAAAGCTTCTAGAAGACGTA
TGGGGAACAGGGGTGAGAGTGACAACTAATGAAGACGGGGTTGTTGAAAGCG
AGGAGATAAGAAGGTGCATCGAAATGGTAATGGGAGGCCGTGATAGTGAATC
AACAATGAGAAAGAATGCTAAGAAGTGGAAGGATGTGGGAAGAGAGGCTATG
AAAGAAACAGGATCTTCTTATATGAATCTCAAGGCTTTTATTAAAGAAGTGAA
TGATGGTGAATCAACCATCAAAACTGAAATTGTTTCAACTATATGA.

In some embodiments, the polynucleotide comprises a nucleic acid sequence with at least 80%, at least 87%, at least 93%, at least 97%, or at least 99% homology or identity to SEQ ID NO: 6, or any value and range therebetween. Each possibility represents a separate embodiment of the invention. In some embodiments, the polynucleotide comprises a nucleic acid sequence with 80% to 98%, 81% to 99%, 85% to 99%, or 80% to 100% homology or identity to SEQ ID NO: 6. Each possibility represents a separate embodiment of the invention.

In some embodiments, the polynucleotide comprises or consists of the nucleic acid sequence:

(SEQ ID NO: 7)
ATGACTAAAATACAACAGCAACCTCACTTTCTCTTAGTAACATATCCCGCA
CAAGGTCATATTAACCCGTCTCTCCGGTTCGCCGAACGACTCATTCGGTTG
GGTGTCAAAGTCACCTTCACAATAACTGTCTCTGCTTACCGCCGAATGAGT
AAAGCGGGCCACATCTCAGAGTTTTTAAATTTTGCTGTTTTTTCAGACGGC
TTTGATGACGGTTTCAACTCCAAAACAGACGATTATGGTCTCTTCTTAACT
CAATTCAGAAGCAGGGGAAAAGATAGCTTGAAAGAAACAATTCTTTCAAAT
GCTAAAAACGGAACTCCAGTTAGTTGTTTGGTTTACACACTCCTACTCCCT
TGGGCTCCTGAGGTGGCACGTGGCCTAAACGTGCCCTCAGCCTTTCTTTGG
ATTCAACCAGCTTCTGTTTTACGACTTTACTATTACTACTTCAATGGGTAC
AATGAACTCATCGGCGACGATTGTAACGAACCTTCATGGTCCATTCAATTA
CCAGGGTTACCATTGCTCAAAAGTCGTGACCTTCCCTCCTTTTGTCTCCCT
TCAAATCCTTACGCTGATGTACTGACTTTAGTCAAAGAGCATTTAGATGTG
TTGGATTTGGAAGAGAAGCCTAAAATACTTGTGAATAGTTTTGATGAGTTG
GAGAGGGAGGCGTTGAATGAAATTGATGGGAAACTAAAAATGGTTGCCGTA
GGGCCTTTGATTCCATCAGCTTTTTTTGGATGGACAGGATGCATCTGA.

In some embodiments, the polynucleotide comprises a nucleic acid sequence with at least 77, at least 85%, at least 95%, or at least 99% homology or identity to SEQ ID NO: 7, or any value and range therebetween. Each possibility represents a separate embodiment of the invention. In some embodiments, the polynucleotide comprises a nucleic acid sequence with 77% to 95%, 82% to 97%, 81% to 98%, or 77% to 100% homology or identity to SEQ ID NO: 7. Each possibility represents a separate embodiment of the invention.

In some embodiments, the polynucleotide comprises or consists of the nucleic acid sequence:

(SEQ ID NO: 8)
ATGGGTTCATGGCGGAATTCAAGAACAACGTCTACAAAGTTTTTATGGTTGATT
TTACCGTTGATGGTGGTGACGGTGATTATAGGGGTAAAAAAGTCAAATTATGG
GTCGAAGTATAATTATCCTTGGGTTTGGAGTTCAGTGATTAATTCTTATTCTTCT
TCTGCGGTTAAAGAAGATGTAACGGTGGTGGCTGAAGGTCCTGTTGAATCATT
TGGGTTGCGGTCAACGGTGGTCAACGGTGGTGGTGTGGTGGCGGAAGGGCCGT
CGGAAGATTTTGGTTTTAATTCTTCTTATCCACCGTTGGCTATGGAAGATGAAA
TGGATGTTGAGCTACCTGCTATTGCCAAGGAAGATGACTTGAACGCGACGTTG
AGTGGACCCGACCTTTTTGTGTCTGCAAATCAAACTGGCGGACTTCATGTTGAT
ATTGGAATCAACAGTAAGTATACCAGTTTGGATAAGCTTGAAGCCCGCTTAGG
TCAGGTTCGAGCTGCAATAAAAGAAGCCGAATCAGGAAATAGAACTTACGATC
CGGATTATGTACCAGAGGGTCCTATGTACTGGCATGCAGCCTCATTTCACAGG
AGTTATTTGGAGATGGAAAAGCAATTTAAGGTGTTTGTATATGAAGAAGGAGA
ACCACCAATATTTCATAACGGTCCTTGCAAAAACATATATGCAATGGAAGGTA
ACTTTATCTACCATATGGAAACAACCAAGTTTAGGACAAAAAACCCCGAAAAA
GCTCACACGTTTTTTCTCCCAATGAGTGCTGCAATGATGGTGAGGTTTATCTTT
GAGCGTGATCCAAATGTTGACCATTGGCGTCCTATGAAGCAAACAATTAAAGA
TTATGTTGATCTTGTGGGTGGTAAGTACCCATTTTGGAATCGAAGCTTAGGAGC
CGATCACTTTACTGTTGCGTGCCACGATTGGGTGAGTAAAGTCTTTTATCCCAT
CATTTTCATGCTTTTACTAGTATTTATCTTCAGAATGTCGACTGGATGCTGA.

In some embodiments, the polynucleotide comprises a nucleic acid sequence with at least 82%, at least 85%, at least 89%, at least 92%, at least 95%, at least 97%, or at least 99% homology or identity to SEQ ID NO: 8, or any value and range therebetween. Each possibility represents a separate embodiment of the invention. In some embodiments, the polynucleotide comprises a nucleic acid sequence with 82% to 95%, 83% to 98%, 82% to 99%, or 82% to 100% homology or identity to SEQ ID NO: 8. Each possibility represents a separate embodiment of the invention.

In some embodiments, the polynucleotide comprises or consists of the nucleic acid sequence:

(SEQ ID NO: 9)
ATGTCAACCGTTGAGGTTGCAAAGTTACTTGTGAATCGAGATCATCGTCTCTTC
ATAACATTCCTTATCATTCAGCCTCCTAGCTCGGGTTCTGGCTCAGCTATCACC
ACCTACATCGAATCATTAGCTGAGAAAGCTATGGACCGCATATCCTTCATTGA
GCTACCTCAAGATAAAATCCCACCACCACGTTACCCGAAATCCCTGCCAACTG
CAGAATCGAAAGCTCATCCCCTTATTTTCATGATTGAGTTCATTAAGTGTCACT
GCAAATATGTTAGAAACATTGTATCTGACATGATAAGTCAACCGAGTTCGGGT
CGGGTAGCTGGGTTGGTAATCGACATGCTTTGTTTCAGCATGATGGATGTCGCT
AATGAGTTCAACATTCCAACCTATGTATTTGTCACTTCTAATGCTGCTTTTCTTG
GATTTTATTTATATGTCCAGATACTCTCTAATGATCAGAACCAAGACGTTGTTG
AGCTGAGCAAATCTGATACCGAGATATCGGTTCCAGGTTTTGTAAAGCCGGTG
CCAACGAAAGTCTTCTGGACTGTTGTCCGCACTAAAGAAGGACTGGACTTTGT
TTTGTCATCTGCCCAGAAACTTAGACAAGCCAAAGCAATCATGGTTAATACCTT
CTTGGAGTTGGAAACACACGCAATCAAGTCGCTGTCTGATGACACCAGCATCC
CGCCTGTGTATCCAGTGGGACCGATACTCAATTTAGAAGGTGGTGCTGGCAAA
ACGTTCGACAATGACATTAGCAGGTGGTTGGACAGTCAACCGCCTTCCTCGGT
GGTGTTCTTGTGCTTTGGAAGCCACGGATGTTTTGATGAGATCCAAGTGAAGG
AGATAGCACATGCTTTAGAGCAGAGTGGCCACCGTTTCTTGTGGTCCCTACGTC
GACCTCCATCAGATCAAACATTAAAAGTTCCCGGTGATTACGAGGATCCAGGA
GTGGTATTACCGGAAGGATTTCTTGAGCGAACTGCTGGACGTGGGAAAGTAAT
TGGGTGGGCCCCGCAGGTGATGGTGCTGGCTCACCGTGCAGTTGGAGGCTTCG
TGTCCCACTGTGGGTGGAACTCGTTGTTGGAGAGTTTGTGGTTCGGCGTACCAA
CGGCAACATGGCCGATCTATGCTGAGCAGCAGATGAATGCGTTTGAAATGGTG
GTGGAGCTGGGACTGGCTGTGGAGATAACATTGGATTATAGGAATGATATGGA
TATGTTCATTGTCACCGCACAGGAGATAGAAAGTGGTATAAGAAAGGTGATGG
AGGATAATGAGGTAAGAACAAAAGTGAAAGAGAGAAGTGAGAAGAGTAGAG
CAGCAGTGGCGGAGGGGGGGTCATCGTATGCATCTGTTGGTCATCTTATTAAA
GAATTTACAGGAAACATCTCCTAA.

In some embodiments, the polynucleotide comprises a nucleic acid sequence with at least 79, at least 85%, at least 95%, or at least 99% homology or identity to SEQ ID NO: 9, or any value and range therebetween. Each possibility represents a separate embodiment of the invention. In some embodiments, the polynucleotide comprises a nucleic acid sequence with 79% to 95%, 82% to 97%, 81% to 98%, or 79% to 100% homology or identity to SEQ ID NO: 9. Each possibility represents a separate embodiment of the invention.

In some embodiments, the polynucleotide comprises or consists of the nucleic acid sequence:

(SEQ ID NO: 10)
ATGTCATCATTCATCAACTTTGTTGAATCCACAACACAACTTCAACCACAATTC
GAACAACTCATCCAAACACTTCTTCCCATAACTGCGATAATATCGGATGGTTTT
TTGATGTGGACACAAGATTCCGCCGAAAAATTCAATATCCCACGTCTGGTTTTT
TATGGGACAAACATATTTTTCATGACTATGTGTAACATTATGGCACAATTTAAG
CCACATGCGGCTGTTAATTCTGATGATGAGGCGTTTGATGTACCCGGTTTCACC
AGGTTTAAGTTGACGGCTAATGATTTTGAGCCGCCTTTTAATGAGGTTGAACCG
AAAGGTTCAATGTTGGATTTTTTATTGGAGCAACAAAAGGCTATGGTTAGGAG
CCATGGGTTGGTGGTTAATAGTTTTTATGAGATTGAACATGAGTTTAATGTTTA
TTGGAATCAGAACTATGGACCTAAAGCTTGGTTAATGGGACCATTTTGTGTAG
CTAAGCCATATGCATCAAACGTCATGGATTCCGAGATATCGACTAAGGTGGTG
AAAAAATCAGCATGGATCCAGTGGCTTGACAGGAAGCTTGCAGCGAACGAGC
CAGTGTTATACATCTCATTTGGAACACAGGCAGAGGCGTCTATGGAGCACTTA
CACGAGGTCGCTATTGGTTTGGAACGATCAAATGTAAGCTTCATTTGGGTGGT
AAAAGCGAAGCAGATGCAATTAATTGGAGCAGGGTTTGAAGAGAGGGTGAAG
GGGAGAGGAAAAGTGGTGACAGAATGGGTGGATCAGATGGAAATCTTGAAAC
ATGAAATTGTAAGCGGGTTTTTAAGTCATTGTGGGTGGAACTCACTGCTAGAG
AGTATGTGTGTGGGTGTGCCGGTGCTTGCAATGCCGTTGATGGCGGATCAACT
CTTAAATGCAAGGTTGGTTGTGGAGGAGATTGGGATGGGGCTACGGTTGTGGC
CGAGGGGTATGGTGGCACGTGGGATAGTTGGGGCGGAGGAAGTCGAGAAAAT
GGTGGTGGAGTTGATGGAAGGGGAAGGTGGGAGAAGGGTGCGGAAAAGGGTC
ATCGAGGTTAGAGAAATGGCATATGGTGCGATGAAGGAAGGAGGGTCATCAT
CGAGGACATTAGACTCGTTGATTGATCATGTTTGTGAAGCCTTTCATAAGACGG
TTTAA.

In some embodiments, the polynucleotide comprises a nucleic acid sequence with at least 78, at least 85%, at least 95%, or at least 99% homology or identity to SEQ ID NO: 10, or any value and range therebetween. Each possibility represents a separate embodiment of the invention. In some embodiments, the polynucleotide comprises a nucleic acid sequence with 78% to 95%, 82% to 97%, 81% to 98%, or 78% to 100% homology or identity to SEQ ID NO: 10. Each possibility represents a separate embodiment of the invention.

In some embodiments, the polynucleotide comprises or consists of the nucleic acid sequence:

(SEQ ID NO: 11)
ATGGGGAGCTTGAAGAAAGGTGCACATATACTAATATTCCCATTCCCAGCACA
AGGTCATATGCTCCCACTCCTAGACCTAACTCACCACCTAGCCACCAATGGGTT
AACCATAACCATATTAGTCACACCCAAAAACCTACCAATCTTGAACCCACTTTT
ATCTTCATCTCCAAACATCCAACCACTAGTCTTCCCTTTCCCACCTCACCCAAG
ACTTCCACCACATGTTGAAAATGTTAAAGACATAGGTAACCATGCAAATGTCC
CAATCACAAACTCACTAGCCAAATTACAAGACCAAATAATCCAGTGGTTTAAC
TCCCACCATAACCCTCCTGTTGCCATCATCTCAGATTTCTTTCTTGGATGGACCC
AACACCTTGCAAACAAACTTGGTATCCCTCGTGTCGGGTTTTTTTCTTCTGGTG
CTTACTTGACTGCTGTTCTTGATTATGTTTGTCATAATATTAAAACTGTTAGGTC
TCAAGAGGAGACTGTTTTTCATGACTTGCCAAATTCTCCTTGTTTTAAATTCGA
GCATCTTCCGGGTTTGGCCCAGATTTATAAAGAGTCCGACCCGGAATGGGAAT
TGGTTCTTGATGGTCATATTGCGAATGGGTTAAGTTGGGGTTGGATTGTGAATA
CTTTTGATGGGTTGGAGTCTCGGTATATGGAGTATCTGACAAAGAAAATGGGT
GTCGGACGGGTTTTTGGTGTCGGGCCAGTTAATTTGTTAAACGGGTCGGATCCC
ATGACCCGTGGGAAATCGGAATCCGGGTCTGATTCCGGTGTGTTGAACTGGCT
CGATGGAAAACCCGATGGGTCGGTTTTGTATGTGTGTTTTGGAAGTCAAAAGT
TTCTTACTAATGACCAAATGGAGGGATTGTCAATTGGGCTTGAACAAAGTGGG
GTCCATTATGTTTGGGTTGTGAAAGACGAACAAGGTGATGCAATTAGGTCCGG
GTCGGGTAGAGGACTAGTGGTAACGGGTTGGGCCCCGCAAGTTTCAATATTGG
GTCATGGAGCGGTGGGTGGGTTTTTGAGTCATTGCGGGTGGAACTCTGTTTTGG
AAGCAATTGTAAATGGAGTTATGATATTGGCTTGGCCAATGGAGGCTGATCAA
TTTGTTAATGCTAAGTTGTTAGTGGATGACCATGGTATAGGGGTGTGGGTTTGT
GAGGGGCCGAATACGGTTCCTGATTCAACCGAGTTGGCTCGTAAAATTGGTGA
GTCAATGAGTACGGATAAGAGTGAGAAGGTAAAGGCGAAAGAAATGAAAAAC
AAAGCAAATGAAGCAGTTAAAGAAGGTGGGAGCTCATCAATGGAATTAAGCA
GGCTTGTTAAGGAGCTGTCTAACTTTGAGACAAATGGGCCATGA.

In some embodiments, the polynucleotide comprises a nucleic acid sequence with at least 82, at least 85%, at least 95%, or at least 99% homology or identity to SEQ ID NO: 11, or any value and range therebetween. Each possibility represents a separate embodiment of the invention. In some embodiments, the polynucleotide comprises a nucleic acid sequence with 82% to 95%, 82% to 97%, 83% to 98%, or 82% to 100% homology or identity to SEQ ID NO: 11. Each possibility represents a separate embodiment of the invention.

In some embodiments, the polynucleotide comprises or consists of the nucleic acid sequence:

(SEQ ID NO: 12)
ATGGATACCCAAACACAAGTCAAGAAACAAAAACTTGAAACCATGGAAC
ATAAAACATCATCCGCCGAAATCTTCGTGCTACCATTTTTTGGTACGGG
TCATATAAACCCAGCAATGGAGCTTTGCCGGAACATTTCATCACATAAT
TACAAAACTACCCTCATCATCCCTTCACATCTTTCTTCATCTATTCCTT
CTCCCTTTTCTTCAACTTTACTTCATGTTGCTGAGATCCCTTTCACTGC
TTCTGACCCGGAACCCGGATCCGGAAGAGGGAACCCACTTGATGCCCAG
AACAAGCAAATGGGTGAAGGGATTAAGGCGTTTATGTCTGCAAGATCTG
ACGGATCAAAACTACCCACGTGTGTTGTTATTGATGTCATGATGAACTG
GAGTAAAGAGATATTTGTTGATTACCAGATTCCTATTGTCTCTTTTTTT
ACTTCTGGAGCTACTAATACTGCTATGGGTTATGGTAGGTGGAAAGCTA
AAATTGGTGATCTGAAGCCCGGGGAGACCCGTGTGATCCCCGGACTTCC
TACTGAAATGGCCGTTACTTTTGCGGATTTAAATCAAGGTCCTAGAGGC
CGTGGGCCTCGGCCGGATGGGTCAAGGCCTGACGGGCCAAGGTCTGGAC
CACCTGGTGGGATGAGGTCCGGACCACCTCACGGGATGAGGGGTGGGGG
ACGAGGTGGGCGGGGCGGTGGACGACCCGGCCCGGATGCGAAACCACGT
TGGGTAGATGAAGTGGACGGGTCGGTAGCTTTGCTTATCAACACGTGTG
ACAATCTCGAGCGTGTGTTTATTGATTACATTGCTGAAGAAACCAAGAT
TCCCGTTTATGGTGTTGGCCCGTTGCTGCCCGAAAAGTATTGGAAGTCA
GCGGGTTCGTTGCTTCGTGATCATGAAATGAGGTCTAACCATAAAGCGA
ATTACTCGGAAGATGAGGTGTTTCAATGGCTAGAATCGAAACCAGTTGG
GTCGGTTATTTACATATCGTTTGGGAGTGAAGTTGGCCCGACTATAGAC
GAGTATAAAGAGTTAGCTGGATCATTGGAAGGATCGAATCAGAATTTCA
TTTGGGTGATCCAGCCCGGTTCGGGGATAACGGGCATGCCAAGATCGTT
TTTGGGCCCGGTTAATACGGATAGTGAGGAAGAAGAGGAAGGGTATTAT
CCTGAGGGATTAGATGTTAAAGTTGGGAACAGGGGTTTGATCATCACTG
GATGGGCTCCACAGTTGTTGATTTTGAGCCACCCATCTACAGGCGGGTT
CTTATCACATTGTGGGTGGAATTCAACTGTTGAGGCGATTGGGCGAGGT
GTTCCGATATTGGGTTGGCCCTTGAGGGGTGATCAGTTTGATAATGCGA
AACTTGTGGCGAATCATTTGAAAATTGGGTTTGCGATGTCAAGTGTGGC
GAGTGAAGGCGGACGACCTGGGAAGTTCAACAAGGAGACTATAACAGCA
GGGATTGAGAAACTAATGAATGATGAAGATGTGCATAAACAGGCAAAGA
AACTTAGTAAAGAATTTGAGAGTGGGTTTCCAGTGAGTTCAGTTAAAGC
ATTGGGTGCTTTCGTGGAGTCTATTAGCCAGAAAGCAACCTAA.

In some embodiments, the polynucleotide comprises a nucleic acid sequence with at least 74, at least 80%, at least 85%, at least 87%, at least 93%, or at least 99% homology or identity to SEQ ID NO: 12, or any value and range therebetween. Each possibility represents a separate embodiment of the invention. In some embodiments, the polynucleotide comprises a nucleic acid sequence with 74% to 95%, 75% to 97%, 76% to 98%, or 74% to 100% homology or identity to SEQ ID NO: 12. Each possibility represents a separate embodiment of the invention.

In some embodiments, the polynucleotide comprises or consists of the nucleic acid sequence:

(SEQ ID NO: 13)
ATGTCACTCGTGACTAATAACCCACATTTACTAGTCTACCCATTACCTA
CCTCCGGCCATATCATTCCGTTACTCGACCTGACCGACCTTCTTCTCCG
CCGTGGCCTCACCATCACCGTCGTGATATCCACCACAGACCTTACGCTT
CTCGACACTCTCCTATCCTCACACCCCACGTCTCTACACAAACTTTACT
TCCCCGACCCCGAAATCGGCCCATCTTCTCATCCCGTTATTGCCAGAAT
AATTGCCACCCAAAAACTATTTGATCCAATTGTTAAATGGTTTGAATCG
CACCCTTCGCCTCCAGTCGCCATCATTTCCGACTTCTTTCTTGGGTGGA
CTAATGAACTTGCATCACGTTTAGGTATTCGACGTGTGGTGTTTTCACC
TTCGGGAGCTCTTGGTCATTCCATTTTACAAAGTTTGTGGCGTGACGTG
GCGGAGATCAATGCAAAAAATGTTGATGGAAATGGAAACTACTCGATTT
CTTTTACCGATATACCAAACTCGCCCGAATTTCATTGGTGGCAGTTGTC
ACAACTTTTGCGTGTTCATAGGGAGGGAGATCCGGACTTCGAATTTTTT
AGGAATGGAATGTTGGCTAATACGAAAAGTTGGGGTATTGTTTACAACA
CATTTGAAAGGATTGAAAAGGTTTACATTGACCATGTGAAGAAACAAAT
AGGTCATGATCGGGTATGGGCAATAGGCCCATTACTTCCCGAAGAACAT
GGCCCAGTTGGTAGCACCGCACGTGGTGGGTCCAGTGTAGTGCCACCTC
ATGACCTTCTCACGTGGTTGGACAAAAAGCCCCATGACTCGGTCGTATA
TATATGTTTTGGGAGTCGATTGACGTTAAGTGAGAAGCAAATGAGTGCA
TTAGCAAGTGCACTCGAGCTCAGTAACGTTGATTTTATTTTGTGTGTGA
AGGCAAGTGGTTCGAGCTTCATTCCTAGTGGGTTCGAAGATCGAGTGGT
GGGTCGGGGGTTCGTAATCAAAGGTTGGGCCCCACAGTTGGCGATATTG
AGACATCGGGCTGTGGGGTCGTTTGTGACTCATTGTGGGTGGAACTCAA
CATTGGAAGGTGTTTCATCAGGAGTGATGATGTTGACGTGGCCAATGGG
TGCAGACCAATATGCAAATGCTAAGCTATTGGTCGACCAGTTAGGTGTT
GGGAAACGAGTTTGTGAAGGTGGACCCGAGAGTGTTCCTGATTCAACTG
AGTTGGCTCGGTTGTTGGAAGAGTCACTGAGTGGTGATACATCCGAGCG
AGTTAAAGTCAAGGAGCTAAGTCGGGAAGCTAACACAGCTGTGAAAGAA
GGAACTTCAATAAGAGATTTRGAACATGTTCGTTAACCTTTTATCCGAG
CTCTAA.

In some embodiments, the polynucleotide comprises a nucleic acid sequence with at least 80, at least 85%, at least 95%, or at least 99% homology or identity to SEQ ID NO: 13, or any value and range therebetween. Each possibility represents a separate embodiment of the invention. In some embodiments, the polynucleotide comprises a nucleic acid sequence with 80% to 95%, 82% to 97%, 81% to 98%, or 80% to 100% homology or identity to SEQ ID NO: 13. Each possibility represents a separate embodiment of the invention.

In some embodiments, the polynucleotide of the invention comprises 700 to 1,800 nucleotides. In some embodiments, the polynucleotide of the invention is 730 to 1,730 nucleotides long.

In some embodiments, 700 to 1,800 nucleotides comprises: at least 705 nucleotides, at least 750 nucleotides, at least 800 nucleotides, at least 900 nucleotides, at least 1,000 nucleotides, at least 1,150 nucleotides, at least 1,400 nucleotides, at least 1,600 nucleotides, at least 1,700 nucleotides, or at least 1,750 nucleotides, or any value and range therebetween. Each possibility represents a separate embodiment of the invention. In some embodiments, 700 to 1,800 nucleotides comprises: 710 to 1,750 nucleotides, 720 to 1,760 nucleotides, 730 to 1,780 nucleotides, or 740 to 1,700 nucleotides. Each possibility represents a separate embodiment of the invention.

In some embodiments, the polynucleotide comprises a plurality of polynucleotides. In some embodiments, the polynucleotide comprises a plurality of types of polynucleotides. As used herein, the term “plurality” comprises any integer equal to or greater than 2. In some embodiments, the polynucleotide comprises at least 2, at least 3, at least 4, at least 5, at least 6, at least 7, at least 8, at least 9, at least 10, at least 11, at least 12, or 13 different nucleic acid sequences, or any value and range therebetween, wherein each of the different nucleic acid sequences is selected from SEQ ID Nos.: 1-13. Each possibility represents a separate embodiment of the invention. In some embodiments, the polynucleotide comprises 2-13, 2-10, 2-8, 2-5, 3-7, 3-9, 3-12, 5-10, 5-12, or 3-13 different nucleic acid sequences, wherein each of the different nucleic acid sequences is selected from SEQ ID Nos.: 1-13.

In some embodiments, the polynucleotide is or comprises a plurality of polynucleotide molecules, wherein each of the plurality of the polynucleotide molecules comprises a different nucleic acid sequence, and wherein the different nucleic acid sequences are selected from SEQ ID Nos.: 1-13.

In some embodiments, the polynucleotide encodes a protein characterized by catalytic activity of transfer a glucuronic acid component of UDP-glucuronic acid to a small hydrophobic molecule (e.g., a UGT). In some embodiments, the polynucleotide encodes a protein characterized by glycosyltransferase catalytic activity. In some embodiments, the polynucleotide encodes a protein characterized by being capable of transferring glucuronic acid component of UDP-glucuronic acid to a cannabinoid or a precursor thereof. In some embodiments, the polynucleotide encodes a protein characterized by having a catalytic activity of glycosylating a cannabinoid or a precursor thereof. In some embodiments, the polynucleotide encodes a UGT enzyme.

In some embodiments, the UGT is a UGT derived from Helichrysum umbraculigerum. As used herein, the term “UGT” encompasses any enzyme derived from H. umbraculigerum and having or characterized by having an activity as described herein.

According to some embodiments, there is provided an artificial nucleic acid molecule comprising the polynucleotide disclosed herein.

In some embodiments, the artificial vector comprises a plasmid. In some embodiments, the artificial vector comprises or is an agrobacterium comprising the artificial nucleic acid molecule. In some embodiments, the artificial vector is an expression vector. In some embodiments, the artificial vector is a plant expression vector. In some embodiments, the artificial vector is for use in expressing a UGT encoding nucleic acid sequence as disclosed herein. In some embodiments, the artificial vector is for use in heterologous expression of a UGT encoding nucleic acid sequence as disclosed herein in a cell, a tissue, or an organism.

Expressing polynucleotide within a cell is well known to one skilled in the art. It can be carried out by, among many methods, transfection, viral infection, or direct alteration of the cell's genome. In some embodiments, the polynucleotide is in an expression vector such as plasmid or viral vector. A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector, a virgaviridae viral vector, or a poxviral vector. The barley stripe mosaic virus (BSMV), the tobacco rattle virus and the cabbage leaf curl geminivirus (CbLCV) may also be used. The promoters may be active in plant cells. The promoters may be a viral promoter.

In some embodiments, the polynucleotide as disclosed herein is operably linked to a promoter. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell). In some embodiments, the promoter is operably linked to the polynucleotide of the invention. In some embodiments, the promoter is a heterologous promoter. In some embodiments, the promoter is the endogenous promoter.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like. The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases.

In some embodiments, the polynucleotide is transcribed by RNA polymerase II (RNAP II and Pol II). RNAP II is an enzyme found in eukaryotic cells, known to catalyze the transcription of DNA to synthesize precursors of mRNA and most snRNA and microRNA.

In some embodiments, a plant expression vector is used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation, and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as systemic infection and targeting specificity, are used for in vivo expression. In one embodiment, systemic infection is inherent in the life cycle of, for example, the retrovirus and is the process by which a single infected cell produces many progeny virions that infect neighboring cells. In one embodiment, the result is that a large area becomes rapidly infected, most of which was not initially infected by the original viral particles. In one embodiment, viral vectors are produced that are unable to spread systemically. In one embodiment, this characteristic can be useful if the desired purpose is to introduce a specified gene into only a localized number of targeted cells.

In some embodiments, plant viral vectors are used. In some embodiments, a wild-type virus is used. In some embodiments, a deconstructed virus such as are known in the art is used. In some embodiments, Agrobacterium is used to introduce the vector of the invention into a virus.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation, agrobacterium Ti plasmids and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the polypeptide), the expression construct of the present invention can also include sequences engineered to optimize stability, production, purification, yield, or activity of the expressed polypeptide.

In some embodiments, the artificial vector comprises a polynucleotide encoding a protein comprising an amino acid sequence as described herein.

According to some embodiments, there is provided a protein encoded by: (a) the polynucleotide disclosed herein; (b) the artificial vector disclosed herein; or the plasmid or agrobacterium disclosed herein.

In some embodiments, the protein is encoded by a polynucleotide comprising or consisting of SEQ ID Nos.: 1-13.

In some embodiments, the protein comprises an amino acid sequence with at least 90%, at least 92%, at least 93%, at least 95%, at least 97%, or at least 99% homology or identity to any one of SEQ ID Nos.: 14-26, or any value and range therebetween. Each possibility represents a separate embodiment of the invention. In some embodiments, the protein comprises an amino acid sequence with 90-100%, 93-100%, 95-100%, or 97-100% homology or identity to any one of SEQ ID Nos.: 14-26. Each possibility represents a separate embodiment of the invention.

In some embodiments, the protein is an isolated protein.

As used herein, the terms “peptide”, “polypeptide” and “protein” are interchangeable and refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides, polypeptides and proteins described have modifications rendering them more stable while in the organism or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the terms “isolated protein” refers to a protein that is essentially free from contaminating cellular components, such as carbohydrate, lipid, or other proteinaceous impurities associated with the nucleic acid in nature. Typically, a preparation of an isolated protein contains the protein in a highly purified form, e.g., at least about 80% pure, at least about 90% pure, at least about 95% pure, greater than 95% pure, or greater than 99% pure. In some embodiments, the isolated protein is a synthesized protein. Synthesis of protein is well known in the art and may be performed, for example, by heterologous expression in a transformed cell, such as exemplified herein.

In some embodiments, the protein comprises or consists of the amino acid sequence:

(SEQ ID NO: 14)
MTNSELVFIPSPGAGHLPPTVELAKLLLHREPQLSVTIIIMNLPHETKP
TTETRMSTPRLRFIDIPKDESTKDLISRHTFISAFLEHQKPHVRNIVRS
ITESDSVRLVGFVVDMFCIAMMDVANELGAPTYLYFTSSAASLGLMFCL
QAKRDDEEFDVTELKDKDSELSIPCYTNPLPAKLLPSVLFDKRGGSKTF
IDLARKYRESRGIVVNTFQELESYAIEYLASSNANVPPVFPVGAILNQE
KKVNDDKTEEIMTWLNEQPESSVVFLCFGSMGSFGEDQIKEIALAIEES
GQRFLWSLRRPPSNENKYPKEYENFGEVLPEGFLERTSSVGKVIGWAPQ
MAVLSHSSVGGFVSHCGWNSTLESIWCGVPVAAWPLYAEQQLNAFKLVV
ELGLAVEIKIDYRSENEIILTSKEIESGIRRLMNDEELRMKVKEMKGNS
RFAVSEGGSSYVSIRRFIDLVMTKE.

In some embodiments, the protein comprises an amino acid sequence with at least 75%, at least 85%, at least 92%, at least 95%, or at least 99% homology or identity to SEQ ID NO: 14, or any value and range therebetween. Each possibility represents a separate embodiment of the invention. In some embodiments, the protein comprises an amino acid sequence with 75% to 99%, 76% to 98%, or 75% to 100% homology or identity to SEQ ID NO: 14. Each possibility represents a separate embodiment of the invention.

In some embodiments, the protein comprises or consists of the amino acid sequence:

(SEQ ID NO: 15)
MPTSELVFIPSPGVGHLSPTIELVNQLLHRDQRLSVTIIVMKFSLESKH
DTETPTSTPRLRFIDIPYDESAMALINPNTFLSAFVEHNKPHVRNIVRD
ISESNSVRLAGFVVDMFCVAMTDVVNEFEIPTYIYFTSTANLLGLMFYL
QAKRDDEGFDVTVLKDSESEFLSVPSYVNPVPAKVLPDAVLDKNGGSQM
CLDLAKGFRESKGIIVNTFQELERRGIEHLLSSNMNLPPVFPVGPILNL
RNAPNDGKTADIMTWLNDHPENSVVFLCFGSMGSFEKEQVKEIAIAIEQ
SGQRFLWSLRRPTSLEKFEFPKDYENPEEVLPKGFLERTKGVGKVIGWA
PQMAVLSHPSVGGFVSHCGWNSTLESIWCGVPIAAWPLYAEQKINAFQL
VVEMGMAAEIRIDYRTNTRPGGGKEMMVMAEEIESGIRKLMSDDEMRKK
VKGMKDKSRAAVLEGGSSHTSIGILIENLVSITI.

In some embodiments, the protein comprises an amino acid sequence with at least 76%, at least 85%, at least 92%, at least 95%, or at least 99% homology or identity to SEQ ID NO: 15, or any value and range therebetween. Each possibility represents a separate embodiment of the invention. In some embodiments, the protein comprises an amino acid sequence with 76% to 99%, 80% to 98%, or 76% to 100% homology or identity to SEQ ID NO: 15. Each possibility represents a separate embodiment of the invention.

In some embodiments, the protein comprises or consists of the amino acid sequence:

(SEQ ID NO: 16)
MVGLKCFWILQKGFRESKGIIVNTFQELERRGIEHLLSSNMDLPPVFPV
GPILNLRNARNDGKMADIMTWLNDQPENSVVFLCFGSRGSFKEEQVKEI
AIAIEQSGQRFLWSLRRPTSIETFEFPKYYENPEEVLPKGFLERTKSVG
KVIGWAPQMAVLSHPSVGGFVSHCGWNSTLESIWCGVPIAAWPLYAEQQ
TNAFQLVVEMGMAAEIRIDYRTNTPLVGGKDMMVTAEEIERGIRKLMSD
DEMRKKVKDMKDKSRGAVLEGGSSHTSIGNLIDVLVSITI.

In some embodiments, the protein comprises an amino acid sequence with at least 77%, at least 85%, at least 92%, at least 95%, or at least 99% homology or identity to SEQ ID NO: 16, or any value and range therebetween. Each possibility represents a separate embodiment of the invention. In some embodiments, the protein comprises an amino acid sequence with 77% to 99%, 79% to 98%, or 77% to 100% homology or identity to SEQ ID NO: 16. Each possibility represents a separate embodiment of the invention.

In some embodiments, the protein comprises or consists of the amino acid sequence:

(SEQ ID NO: 17)
MATNNLHFLLIPHIGPGHTIPMIDMAKLLAKQPNVMVTIATTPLNITRY
GHTLADAINSFRFFEVPFPAVEAGLPEGCESTDKIPSMDLVPNFLTAIG
MLEQKLEEHFHLLEPRPNCIISDKYMSWTGDFADKYRIPRIMFDGMSCF
NELCYNNLYENKVFEGMHETEPFVVPGLPDKIELTRKQLPPEFNPSSID
TSEFRQRARDAEVRAYGVVINSFEELEQEYVNEYKKLRKGKVWCIGPLS
LCNSDNSDKAQRGNIASVDEEKCLKWLDSHEADSVVYACFGSLVRVNTP
QLIELGLGLEASNRPFIWVVRSVHREKEVEEWLVESGFEERIKDRGLII
RGWAPQVLILSHPSIGGFLTHCGWNSTLESVCAGVPMITWPQFAEQFIN
EKLIVQVLGIGVGVGVDSVVHVGEEDRSGVKVKRESVTKAIEKVMDDEI
DGNERRRRSKEFGKIANNAIKEGGSSYLNLTLLIQDIMRYANADASS.

In some embodiments, the protein comprises an amino acid sequence with at least 88%, at least 92%, at least 95%, or at least 99% homology or identity to SEQ ID NO: 17, or any value and range therebetween. Each possibility represents a separate embodiment of the invention. In some embodiments, the protein comprises an amino acid sequence with 88% to 100%, 90% to 100%, or 95% to 100% homology or identity to SEQ ID NO: 17. Each possibility represents a separate embodiment of the invention.

In some embodiments, the protein comprises or consists of the amino acid sequence:

(SEQ ID NO: 18)
MEKTPHIAIVPSPGMGHLIPLVEFAKKLKNHHNIHATFIIPNDGPLSIS
QKVELDSLPNGLNYLILPPVNFDDLPQDTQIETRISLMVTRSLDSLREV
FKSLVVEKNMVALFIDLFGTDAFDVAIEFGVSPYVFFPSTAMALSLFLY
LPKLDQMVSCEYRELPEPVQIPGCIPVRGQDLVDPVQDRKNDAYKWVLH
NAKKYSMAKGIAVNSFKELEGGALNALLEDEPGKPKVYPVGPLVQTGFS
CDVDSIECLKWLDGQPCGSVLYISFGSGGTLSSSQLNELAMGLELSEQR
FIWVVRSPNDQPNATYFDSHGHKDPLGFLPKGFLERTKGIGFVIPSWAP
QAQILSHSATGGFLTHCGWNSILETVVHGVPVIAWPLYAEQKMNAVSLT
EGIKMALRPTVGENGIVGRLEVARVVKSLLEGEEGKAIRSRVRDLKDAA
ANVLSKDGSSTKTLDQLAVQLKKQELS.

In some embodiments, the protein comprises an amino acid sequence with at least 90%, at least 95%, or at least 99% homology or identity to SEQ ID NO: 18, or any value and range therebetween. Each possibility represents a separate embodiment of the invention. In some embodiments, the protein comprises an amino acid sequence with 90% to 100%, 93% to 100%, 95% to 100%, or 97% to 100% homology or identity to SEQ ID NO: 18. Each possibility represents a separate embodiment of the invention.

In some embodiments, the protein comprises or consists of the amino acid sequence:

(SEQ ID NO: 19)
MTQKQMQMQPHFLLVTYPAQGHINPSLQFAERLIRLGVKVTFTTTVSAY
RRMSKAGNISEFLNFAAFSDGFDDGFNFETDDHGLFLTQLRSRGKDSLK
ETILSNAKNGTPISCLVYTLLLPWAPEVARGLNVPSAFLWIQPASVLRL
YYYYFNGYNELIGDDCNEPSWSIQLPGLPLLKS.

In some embodiments, the protein comprises an amino acid sequence with at least 77%, at least 85%, at least 92%, at least 95%, or at least 99% homology or identity to SEQ ID NO: 19, or any value and range therebetween. Each possibility represents a separate embodiment of the invention. In some embodiments, the protein comprises an amino acid sequence with 77% to 100%, 79% to 100%, 80% to 100%, or 90% to 100% homology or identity to SEQ ID NO: 19. Each possibility represents a separate embodiment of the invention.

In some embodiments, the protein comprises or consists of the amino acid sequence:

(SEQ ID NO: 20)
MTKIQQQPHFLLVTYPAQGHINPSLRFAERLIRLGVKVTFTITVSAYRR
MSKAGHISEFLNFAVFSDGFDDGFNSKTDDYGLFLTQFRSRGKDSLKET
ILSNAKNGTPVSCLVYTLLLPWAPEVARGLNVPSAFLWIQPASVLRLYY
YYFNGYNELIGDDCNEPSWSIQLPGLPLLKSRDLPSFCLPSNPYADVLT
LVKEHLDVLDLEEKPKILVNSFDELEREALNEIDGKLKMVAVGPLIPSA
FFGWTGCI.

In some embodiments, the protein comprises an amino acid sequence with at least 73%, at least 85%, at least 92%, at least 95%, or at least 99% homology or identity to SEQ ID NO: 20, or any value and range therebetween. Each possibility represents a separate embodiment of the invention. In some embodiments, the protein comprises an amino acid sequence with 73% to 100%, 77% to 100%, 85% to 100%, or 90% to 100% homology or identity to SEQ ID NO: 20. Each possibility represents a separate embodiment of the invention.

In some embodiments, the protein comprises or consists of the amino acid sequence:

(SEQ ID NO: 21)
MGSWRNSRTTSTKFLWLILPLMVVTVIIGVKKSNYGSKYNYPWVWSSVI
NSYSSSAVKEDVTVVAEGPVESFGLRSTVVNGGGVVAEGPSEDFGFNSS
YPPLAMEDEMDVELPAIAKEDDLNATLSGPDLFVSANQTGGLHVDIGIN
SKYTSLDKLEARLGQVRAAIKEAESGNRTYDPDYVPEGPMYWHAASFHR
SYLEMEKQFKVFVYEEGEPPIFHNGPCKNIYAMEGNFIYHMETTKFRTK
NPEKAHTFFLPMSAAMMVRFIFERDPNVDHWRPMKQTIKDYVDLVGGKY
PFWNRSLGADHFTVACHDWVSKVFYPIIFMLLLVFIFRMSTGC.

In some embodiments, the protein comprises an amino acid sequence with at least 81%, at least 90%, at least 95%, or at least 99% homology or identity to SEQ ID NO: 21, or any value and range therebetween. Each possibility represents a separate embodiment of the invention. In some embodiments, the protein comprises an amino acid sequence with 81% to 100%, 85% to 100%, 87% to 100%, or 91% to 100% homology or identity to SEQ ID NO: 21. Each possibility represents a separate embodiment of the invention.

In some embodiments, the protein comprises or consists of the amino acid sequence:

(SEQ ID NO: 22)
MSTVEVAKLLVNRDHRLFITFLIIQPPSSGSGSAITTYIESLAEKAMDR
ISFIELPQDKIPPPRYPKSLPTAESKAHPLIFMIEFIKCHCKYVRNIVS
DMISQPSSGRVAGLVIDMLCFSMMDVANEFNIPTYVFVTSNAAFLGFYL
YVQILSNDQNQDVVELSKSDTEISVPGFVKPVPTKVFWTVVRTKEGLDF
VLSSAQKLRQAKAIMVNTFLELETHAIKSLSDDTSIPPVYPVGPILNLE
GGAGKTFDNDISRWLDSQPPSSVVFLCFGSHGCFDEIQVKEIAHALEQS
GHRFLWSLRRPPSDQTLKVPGDYEDPGVVLPEGFLERTAGRGKVIGWAP
QVMVLAHRAVGGFVSHCGWNSLLESLWFGVPTATWPIYAEQQMNAFEMV
VELGLAVEITLDYRNDMDMFIVTAQEIESGIRKVMEDNEVRTKVKERSE
KSRAAVAEGGSSYASVGHLIKEFTGNIS.

In some embodiments, the protein comprises an amino acid sequence with at least 74%, at least 85%, at least 92%, at least 95%, or at least 99% homology or identity to SEQ ID NO: 22, or any value and range therebetween. Each possibility represents a separate embodiment of the invention. In some embodiments, the protein comprises an amino acid sequence with 74% to 100%, 79% to 100%, 85% to 100%, or 90% to 100% homology or identity to SEQ ID NO: 22. Each possibility represents a separate embodiment of the invention.

In some embodiments, the protein comprises or consists of the amino acid sequence:

(SEQ ID NO: 23)
MSSFINFVESTTQLQPQFEQLIQTLLPITAIISDGFLMWTQDSAEKFNI
PRLVFYGTNIFFMTMCNIMAQFKPHAAVNSDDEAFDVPGFTRFKLTAND
FEPPFNEVEPKGSMLDFLLEQQKAMVRSHGLVVNSFYEIEHEFNVYWNQ
NYGPKAWLMGPFCVAKPYASNVMDSEISTKVVKKSAWIQWLDRKLAANE
PVLYISFGTQAEASMEHLHEVAIGLERSNVSFIWVVKAKQMQLIGAGFE
ERVKGRGKVVTEWVDQMEILKHEIVSGFLSHCGWNSLLESMCVGVPVLA
MPLMADQLLNARLVVEEIGMGLRLWPRGMVARGIVGAEEVEKMVVELME
GEGGRRVRKRVIEVREMAYGAMKEGGSSSRTLDSLIDHVCEAFHKTV.

In some embodiments, the protein comprises an amino acid sequence with at least 76%, at least 85%, at least 92%, at least 95%, or at least 99% homology or identity to SEQ ID NO: 23, or any value and range therebetween. Each possibility represents a separate embodiment of the invention. In some embodiments, the protein comprises an amino acid sequence with 76% to 100%, 80% to 100%, 90% to 100%, or 95% to 100% homology or identity to SEQ ID NO: 23. Each possibility represents a separate embodiment of the invention.

In some embodiments, the protein comprises or consists of the amino acid sequence:

(SEQ ID NO: 24)
MGSLKKGAHILIFPFPAQGHMLPLLDLTHHLATNGLTITILVTPKNLPI
LNPLLSSSPNIQPLVFPFPPHPRLPPHVENVKDIGNHANVPITNSLAKL
QDQIIQWFNSHHNPPVAIISDFFLGWTQHLANKLGIPRVGFFSSGAYLT
AVLDYVCHNIKTVRSQEETVFHDLPNSPCFKFEHLPGLAQIYKESDPEW
ELVLDGHIANGLSWGWIVNTFDGLESRYMEYLTKKMGVGRVFGVGPVNL
LNGSDPMTRGKSESGSDSGVLNWLDGKPDGSVLYVCFGSQKFLTNDQME
GLSIGLEQSGVHYVWVVKDEQGDAIRSGSGRGLVVTGWAPQVSILGHGA
VGGFLSHCGWNSVLEAIVNGVMILAWPMEADQFVNAKLLVDDHGIGVWV
CEGPNTVPDSTELARKIGESMSTDKSEKVKAKEMKNKANEAVKEGGSSS
MELSRLVKELSNFETNGP.

In some embodiments, the protein comprises an amino acid sequence with at least 81%, at least 92%, at least 95%, or at least 99% homology or identity to SEQ ID NO: 24, or any value and range therebetween. Each possibility represents a separate embodiment of the invention. In some embodiments, the protein comprises an amino acid sequence with 81% to 100%, 85% to 100%, 90% to 100%, or 93% to 100% homology or identity to SEQ ID NO: 24. Each possibility represents a separate embodiment of the invention.

In some embodiments, the protein comprises or consists of the amino acid sequence:

(SEQ ID NO: 25)
MDTQTQVKKQKLETMEHKTSSAEIFVLPFFGTGHINPAMELCRNISSHN
YKTTLIIPSHLSSSIPSPFSSTLLHVAEIPFTASDPEPGSGRGNPLDAQ
NKQMGEGIKAFMSARSDGSKLPTCVVIDVMMNWSKEIFVDYQIPIVSFF
TSGATNTAMGYGRWKAKIGDLKPGETRVIPGLPTEMAVTFADLNQGPRG
RGPRPDGSRPDGPRSGPPGGMRSGPPHGMRGGGRGGRGGGRPGPDAKPR
WVDEVDGSVALLINTCDNLERVFIDYIAEETKIPVYGVGPLLPEKYWKS
AGSLLRDHEMRSNHKANYSEDEVFQWLESKPVGSVIYISFGSEVGPTID
EYKELAGSLEGSNQNFIWVIQPGSGITGMPRSFLGPVNTDSEEEEEGYY
PEGLDVKVGNRGLIITGWAPQLLILSHPSTGGFLSHCGWNSTVEAIGRG
VPILGWPLRGDQFDNAKLVANHLKIGFAMSSVASEGGRPGKFNKETITA
GIEKLMNDEDVHKQAKKLSKEFESGFPVSSVKALGAFVESISQKAT.

In some embodiments, the protein comprises an amino acid sequence with at least 71%, at least 85%, at least 92%, at least 95%, or at least 99% homology or identity to SEQ ID NO: 25, or any value and range therebetween. Each possibility represents a separate embodiment of the invention. In some embodiments, the protein comprises an amino acid sequence with 71% to 100%, 77% to 100%, 85% to 100%, or 90% to 100% homology or identity to SEQ ID NO: 25. Each possibility represents a separate embodiment of the invention.

In some embodiments, the protein comprises or consists of the amino acid sequence:

(SEQ ID NO: 26)
MSLVTNNPHLLVYPLPTSGHIIPLLDLTDLLLRRGLTITVVISTTDLTL
LDTLLSSHPTSLHKLYFPDPEIGPSSHPVIARIIATQKLFDPIVKWFES
HPSPPVAIISDFFLGWTNELASRLGIRRVVFSPSGALGHSILQSLWRDV
AEINAKNVDGNGNYSISFTDIPNSPEFHWWQLSQLLRVHREGDPDFEFF
RNGMLANTKSWGIVYNTFERIEKVYIDHVKKQIGHDRVWAIGPLLPEEH
GPVGSTARGGSSVVPPHDLLTWLDKKPHDSVVYICFGSRLTLSEKQMSA
LASALELSNVDFILCVKASGSSFIPSGFEDRVVGRGFVIKGWAPQLAIL
RHRAVGSFVTHCGWNSTLEGVSSGVMMLTWPMGADQYANAKLLVDQLGV
GKRVCEGGPESVPDSTELARLLEESLSGDTSERVKVKELSREANTAVKE
GTSIRDLNMFVNLLSEL.

In some embodiments, the protein comprises an amino acid sequence with at least 78%, at least 85%, at least 92%, at least 95%, or at least 99% homology or identity to SEQ ID NO: 26, or any value and range therebetween. Each possibility represents a separate embodiment of the invention. In some embodiments, the protein comprises an amino acid sequence with 78% to 100%, 85% to 100%, 90% to 100%, or 93% to 100% homology or identity to SEQ ID NO: 26. Each possibility represents a separate embodiment of the invention.

In some embodiments, the protein comprises an amino acid sequence set forth in SEQ ID Nos: 14-15, 17-20, 24, or 26.

In some embodiments, the protein comprises an amino acid sequence set forth in SEQ ID Nos: 14, 19, 24, or 26.

The terms “homology” or “identity”, as used interchangeably herein, refer to sequence identity between two amino acid sequences or two nucleic acid sequences, with identity being a stricter comparison. The phrases “percent identity or homology” and “% identity or homology” refer to the percentage of sequence identity found in a comparison of two or more amino acid sequences or nucleic acid sequences. Two or more sequences can be anywhere from 0-100% identical, or any value there between. Identity can be determined by comparing a position in each sequence that can be aligned for purposes of comparison to a reference sequence. When a position in the compared sequence is occupied by the same nucleotide base or amino acid, then the molecules are identical at that position. A degree of identity of amino acid sequences is a function of the number of identical amino acids at positions shared by the amino acid sequences. A degree of identity between nucleic acid sequences is a function of the number of identical or matching nucleotides at positions shared by the nucleic acid sequences. A degree of homology of amino acid sequences is a function of the number of amino acids at positions shared by the polypeptide sequences.

The following is a non-limiting example for calculating homology or sequence identity between two sequences (the terms are used interchangeably herein). The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In some embodiments, % homology or identity as described herein are calculated or determined using the basic local alignment search tool (BLAST). In some embodiments, % homology or identity as described herein are calculated or determined using Blossum 62 scoring matrix.

According to another embodiments, there is provided a compound and/or a salt thereof, and/or a decarboxylated derivative thereof, wherein the compound comprises a glycosylated cannabinoid and/or a glycosylated cannabinoid precursor (such as a glycosylated OA). In some embodiments, the compound of the invention is an isolated compound. In some embodiments, the compound of the invention is a natural or a synthetic compound. In some embodiments, the compound of the invention is a single compound or a plurality of chemically distinct compounds.

As used herein, the term “isolated compound” refers to a compound that is essentially free from contaminating cellular components, such as carbohydrate, lipid, or other proteinaceous impurities associated with the nucleic acid in nature. Typically, a preparation of an isolated compound contains the compound in a highly purified form, e.g., at least about 80% pure, at least about 90% pure, at least about 95% pure, greater than 95% pure, or greater than 99% pure.

In some embodiments, the compound of the invention is chemically pure (e.g. being substantially devoid of one or more impurity, wherein the impurity comprises any organic compound). In some embodiments, the compound of the invention is characterized by a chemical purity of at least 70%, at least 80%, at least 90%, at least 93%, at least 94%, at least 95%, at least 96%, at least 97%, at least 98%, at least 99%, including any range between. In some embodiments, the compound of the invention is characterized by a chemical purity of at most 99.99%, at most 99.9%, at most 99%, at most 95%, at most 90%, including any range between.

In some embodiments, the glycosylated cannabinoid of the invention is represented by Formula 1:

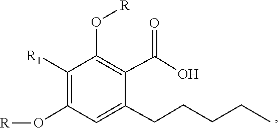

### or by Formula 2:

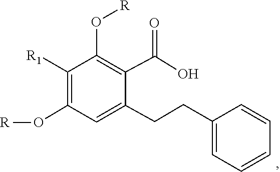

including any salt and/or a decarboxylated derivative thereof; wherein: each R is independently H or a sugar moiety; and R1 is

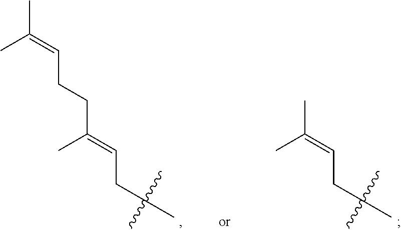

and wherein at least one R is the sugar moiety. In some embodiments, the sugar moiety is or comprises a deoxy monosaccharide, or a deoxy disaccharide. In some embodiments, the term “deoxy” refers to a monosaccharide or a disaccharide having a bond instead of one of the hydroxy groups (i.e. a monosaccharide or a disaccharide devoid of one of the hydroxy groups thereof). In some embodiments, the sugar moiety is or comprises a deoxyhexose. In some embodiments, the sugar moiety is or comprises a deoxyglucose (e.g. 2-deoxy-D-glucose, and/or an enantiomer thereof).

In some embodiments, the glycosylated cannabinoid of the invention is or comprises any one of:

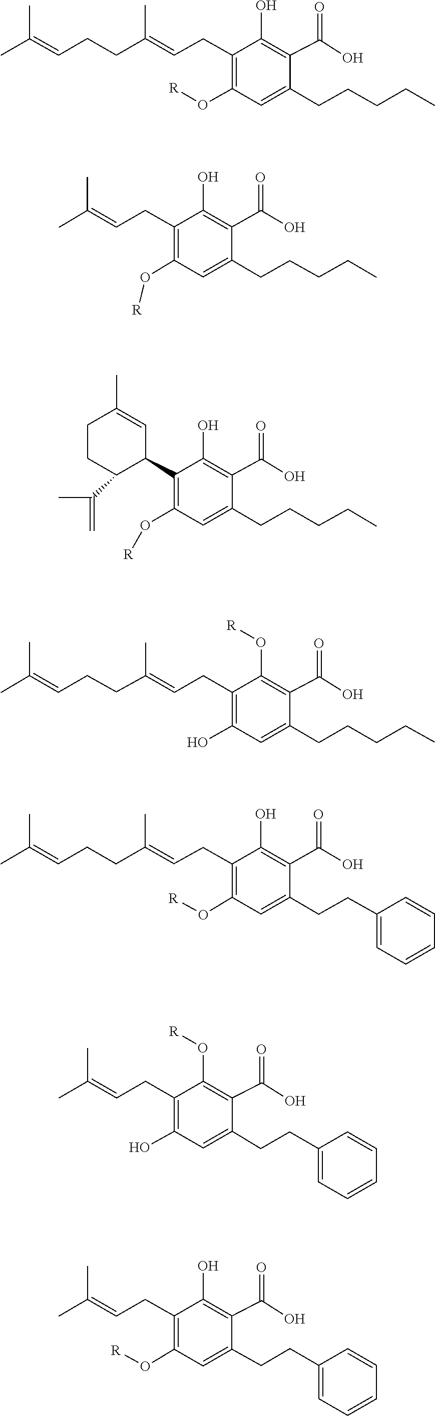

including any salt and/or a decarboxylated derivate thereof, wherein R is as described herein. In some embodiments, R is

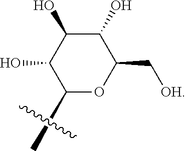

According to some embodiments, there is provided a transgenic cell comprising: (a) the polynucleotide disclosed herein; (b) the artificial nucleic acid molecule disclosed herein; (c) the plasmid or agrobacterium disclosed herein; (d) the isolated protein disclosed herein; or any combination thereof.

As used herein, the term “transgenic cell” refers to any cell that has undergone human manipulation on the genomic or gene level. In some embodiments, the transgenic cell has had exogenous polynucleotide, such as an isolated DNA molecule as disclosed herein, introduced into it. In some embodiments, a transgenic cell comprises a cell that has an artificial vector introduced into it. In some embodiments, a transgenic cell is a cell which has undergone genome mutation or modification. In some embodiments, a transgenic cell is a cell that has undergone CRISPR genome editing. In some embodiments, a transgenic cell is a cell that has undergone targeted mutation of at least one base pair of its genome. In some embodiments, the exogenous polynucleotide (e.g., the isolated DNA molecule disclosed herein) or vector is stably integrated into the cell. In some embodiments, the transgenic cell expresses a polynucleotide of the invention. In some embodiments, the transgenic cell expresses a vector of the invention. In some embodiments, the transgenic cell expresses a protein of the invention. In some embodiments, the transgenic cell, is a cell that is devoid of a polynucleotide of the invention that has been transformed or genetically modified to include the polynucleotide of the invention. In some embodiments, CRISPR technology is used to modify the genome of the cell, as described herein.

In some embodiments, the cell comprises: a unicellular organism, a cell of a multicellular organism, or a cell in a culture.

In some embodiments, a unicellular organism comprises a fungus or a bacterium.

In some embodiments, the fungus is a yeast cell.

In some embodiments, the cell is an arthropod cell. In some embodiments, the cell is an insect cell. In some embodiments, the cell comprises an insect cell line.

Types of insect cell lines suitable for transformation and/or heterologous expression are common and would be apparent to one of ordinary skill in the art. Non-limiting examples of such insect cell lines include, but are not limited to, Sf-9 cells, SR+ Schneider cells, S2 cells, and others.

According to some embodiments, there is provided an extract derived from a transgenic cell disclosed herein, or any fraction thereof.

In some embodiments, the extract comprises the polynucleotide of the invention, an isolated DNA molecule as disclosed herein, an isolated protein as disclosed herein, or any combination thereof.

According to some embodiments, there is provided a homogenate, lysate, extract, derived from a transgenic cell disclosed herein, any combination thereof, or any fraction thereof.

Methods and/or means for extracting, lysing, homogenizing, fractionating, or any combination thereof, a cell or a culture of same, are common and would be apparent to one of ordinary skill in the art of cell biology and biochemistry. Non-limiting examples include, but are not limited to, pressure lysis (e.g., such as using a French press), enzymatic lysis, soluble-insoluble phase separation (such for obtaining a supernatant and a pellet), detergent-based lysis, solvent (e.g., polar, or nonpolar solvent), liquid chromatography mass spectrometry, or others.

According to some embodiments, there is provided a transgenic plant, a transgenic plant tissue or a plant part. In some embodiments, there is provided a transgenic plant, or any portion, seed, tissue, or organ thereof, comprising at least one transgenic plant cell of the invention. In some embodiments, the transgenic plant, transgenic plant tissue or plant part, comprises: (a) the polynucleotide disclosed herein; (b) the artificial disclosed herein; (c) the plasmid or agrobacterium disclosed herein; (d) the isolated protein of the invention; (e) the transgenic cell disclosed herein; or any combination thereof.

In some embodiments, the transgenic plant, transgenic plant tissue, or plant part consists of transgenic plant cells of the invention. In some embodiments, the transgenic plant, transgenic plant tissue, or plant part comprises at least: 20%, 25%, 30%, 35%, 40%, 45%, 50%, 55%, 60%, 65%, 70%, 75%, 80%, 85%, 90%, 95%, 97%, or 99% transgenic cells of the invention, or any value and range therebetween. Each possibility represents a separate embodiment of the invention. In some embodiments, the transgenic plant, transgenic plant tissue, or plant part comprises 20%-50%, 20%-60%, 20%-70%, 20%-80%, 20%-90%, or 20%-100% transgenic cells of the invention. Each possibility represents a separate embodiment of the invention.

In some embodiments, the transgenic plant, transgenic plant tissue, or plant part is or derived from a Cannabis sativa plant. In some embodiments, the transgenic plant is a C. sativa plant.

In some embodiments, the transgenic plant, transgenic plant tissue, or plant part is or derived from hemp. In some embodiments, C. sativa comprises or is hemp.

According to some embodiments, there is provided a composition comprising any one of the herein disclosed: (a) polynucleotide of the invention (for example, an isolated DNA molecule); (b) artificial vector; (c) plasmid or agrobacterium; (d) isolated protein of the invention; (e) transgenic cell; (f) extract; (g) transgenic plant tissue or plant part; and (h) any combination of (a) to (g), and an acceptable carrier.

As used herein, the terms “carrier”, “excipient”, or “adjuvant” refers to any component of a composition, e.g., pharmaceutical or nutraceutical, that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers, and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers, and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

### Methods of Synthesis

According to some embodiments, there is provided a method for synthesizing a glycosylated cannabinoid or a precursor thereof. According to some embodiments, there is provided a method for synthesizing a glucosylated cannabinoid or a precursor thereof.

According to some embodiments, there is provided a method for glycosylating a cannabinoid or a precursor thereof.

In some embodiments, the method comprises synthesizing a monoglycosylated cannabinoid or a precursor thereof. In some embodiments, the method comprises monoglycosylating a cannabinoid or a precursor thereof.

In some embodiments, glycosylating comprises glucosylating. In some embodiments, glucosylating comprises adding glucose to a cannabinoid or a precursor thereof. In some embodiments, a cannabinoid, or a precursor thereof, according to the disclosed method, comprises glucose. In some embodiments, glycosylating comprises monoglycosylating. In some embodiments, glycosylating comprises diglycosylating. In some embodiments, a cannabinoid, or a precursor thereof, according to the disclosed method, is monoglycosylated. In some embodiments, a cannabinoid, or a precursor thereof, according to the disclosed method, is diglycosylated.

According to some embodiments, the method comprises the steps: (a) providing a cell comprising an artificial vector comprising a nucleic acid sequence having at least 87%, at least 89%, at least 92%, at least 95%, at least 97%, or at least 99% homology or identity to any one of SEQ ID Nos.: 1-13, or any combination thereof, or any value and range therebetween; and (b) culturing the cell from step (a) such that a protein encoded by the artificial vector is expressed, thereby synthesizing a glycosylated cannabinoid or a precursor thereof. Each possibility represents a separate embodiment of the invention.

According to some embodiments, the method comprises the steps: (a) providing a cell comprising an artificial vector comprising a nucleic acid sequence having at least 87%, at least 89%, at least 92%, at least 95%, at least 97%, or at least 99% homology or identity to any one of SEQ ID Nos.: 1-13, or any combination thereof, or any value and range therebetween; and (b) culturing the cell from step (a) such that a protein encoded by the artificial vector is expressed, thereby glycosylating a cannabinoid or a precursor thereof. Each possibility represents a separate embodiment of the invention.

According to some embodiments, the method comprises contacting a cannabinoid or a precursor thereof with an effective amount of a protein comprising an amino acid sequence with at least 90%, at least 93%, at least 95%, at least 99%, or 100% homology or identity to any one of SEQ ID Nos.: 14-26, or any value and range therebetween, thereby glycosylating a cannabinoid or a precursor thereof. Each possibility represents a separate embodiment of the invention.

According to some embodiments, the method comprises contacting a cannabinoid or a precursor thereof with an effective amount of a protein comprising an amino acid sequence with at least 90%, at least 93%, at least 95%, at least 99%, or 100% homology or identity to any one of SEQ ID Nos.: 14-26, or any value and range therebetween, thereby synthesizing a glycosylated cannabinoid or a precursor thereof. Each possibility represents a separate embodiment of the invention.

In some embodiments, the cannabinoid is or comprises: CBDA, CBGA, HeliCBGA, delta-9-tetrahydrocannabinolic acid (Δ⁹-THCA), Δ⁹-THC, CBD, CBG, CBCA, or any combination thereof.

In some embodiments, the cannabinoid precursor is or comprises olivetolic acid (OA). In some embodiments, the cannabinoid precursor is or comprises any of: olivetol, DHSA, HA, iValA, BA, and VA, including any salt and any combination thereof.

According to some embodiments, there is provided a method for synthesizing a glycosylated or glucosylated phloroglucinoid, flavonoid, or any precursor thereof.

According to some embodiments, there is provided a method for glycosylating phloroglucinoid, flavonoid, or any precursor thereof.

According to some embodiments, the method comprises the steps: (a) providing a cell comprising an artificial vector comprising a nucleic acid sequence having at least 87%, at least 89%, at least 92%, at least 95%, at least 97%, or at least 99% homology or identity to any one of SEQ ID Nos.: 1-13, or any combination thereof, or any value and range therebetween; and (b) culturing the cell from step (a) such that a protein encoded by the artificial vector is expressed, thereby synthesizing a glycosylated phloroglucinoid, flavonoid, or any precursor thereof. Each possibility represents a separate embodiment of the invention.

According to some embodiments, the method comprises contacting phloroglucinoid, flavonoid, or any precursor thereof with an effective amount of a protein comprising an amino acid sequence with at least 90%, at least 93%, at least 95%, at least 99%, or 100% homology or identity to any one of SEQ ID Nos.: 14-26, or any value and range therebetween, thereby synthesizing a glycosylated phloroglucinoid, flavonoid, or any precursor thereof. Each possibility represents a separate embodiment of the invention.

In some embodiments, a phloroglucinoid, flavonoid, or any precursor thereof is selected from: 1-(2,4,6-trihydroxyphenylhexan)-1-one, naringenin chalcone, pinocembrin chalcone, or any combination thereof.

According to some embodiments, there is provided a method for obtaining an extract from a transgenic cell or a transfected cell.

In some embodiments, the method comprises culturing a transgenic cell or a transfected cell in a medium and extracting the transgenic cell or the transfected cell.

In some embodiments, the method comprises the steps: (a) culturing a transgenic cell or a transfected cell in a medium; and (b) extracting the transgenic cell or the transfected cell, thereby obtaining an extract from the transgenic cell or the transfected cell.

In some embodiments, the transgenic cell or the transfected cell comprises an artificial vector comprising a nucleic acid sequence having at least 87%, at least 89%, at least 92%, at least 95%, at least 97%, at least 99%, or 100% homology or identity to any one of SEQ ID Nos.: 1-13, or any combination thereof, or any value and range therebetween. Each possibility represents a separate embodiment of the invention.

In some embodiments, the transgenic cell or the transfected cell comprises the polynucleotide of the invention or a plurality thereof, as disclosed herein.

In some embodiments, the transgenic cell or the transfected cell comprises the artificial nucleic acid molecule or vector as disclosed herein.

In some embodiments, the cell is a transgenic cell, or a cell transfected with an isolated DNA molecule as disclosed herein.

In some embodiments, the culturing comprises supplementing the cell with an effective amount of a cannabinoid or a precursor thereof. In some embodiments, the supplementing is via the growth or culture medium wherein the cell is cultured.

In some embodiments, the method further comprises a step preceding step (a), comprising introducing or transfecting the cell with the artificial nucleic acid molecule or vector, disclosed herein.

Method for introducing or transfecting a cell with an artificial nucleic acid molecule or vector are common and would be apparent to one of ordinary skill in the art.

In some embodiments, introducing or transfecting comprises transferring an artificial nucleic acid molecule or vector comprising the polynucleotide disclosed herein into a cell; or modifying the genome of a cell to include the polynucleotide disclosed herein. In some embodiments, the transferring comprises transfection. In some embodiments, the transferring comprises transformation. In some embodiments, the transferring comprises lipofection. In some embodiments, the transferring comprises nucleofection. In some embodiments, the transferring comprises viral infection.

As used herein, the terms “transfecting” and “introducing” are interchangeable.

In some embodiments, the contacting is in a cell-free system.

Types of suitable cell-free systems for utilizing any one of: the polynucleotide of the invention or a plurality thereof, as disclosed herein, and the isolated protein of the invention, or a plurality thereof, would be apparent to one of ordinary skill in the art.

In some embodiments, the method further comprises a step preceding step (b), comprising separating the cultured transgenic cell or the cultured transfected cell from the medium.

Methods for separating cell from a medium are common and may include, but not limited to, centrifugation, ultracentrifugation, or other, as would be apparent to one of ordinary skill in the art.

According to some embodiments, there is provided an extract of a transgenic cell, or a transfected cell obtained according to the herein disclosed method.

According to some embodiments, there is provided a medium or a portion thereof separated from a cultured transgenic cell or a cultured transfected cell, obtained according to the herein disclosed method.

According to some embodiments, there is provided a composition comprising: (a) the extract disclosed herein; (b) the medium disclosed herein or a portion thereof; or (c) any combination of (a) and (b), and an acceptable carrier, as described herein.

In some embodiments, a portion comprises a fraction or a plurality thereof.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Chemicals

CBGA, hexanoic-D₁₁ acid (D>98%), and uridine 5′-diphosphoglucose (UDP) disodium salt were purchased from Sigma-Aldrich (Rehovot, Israel). HeliCBGA (NP009525, 90%) was purchased from Analyticon Discovery GmbH (Potsdam, Germany). OA was purchased from Cayman Chemical (Ann Arbor, MI, USA). Phenylalanine-D₅ (D>98%) and phenylalanine-¹³C₉,¹⁵N₁ (¹³C,¹⁵N>99%) were synthesized by Cambridge Isotope Laboratories (Andover, MA). Pentanoic-D₉ acid (D>98%), heptanoic-D₅ acid (D>99%) and iso-caproic-D₁₁ acid (D>98%) were purchased from C/D/N isotopes (Quebec, Canada). Naringenin chalcone, pinocembrin chalcone and hexanoylphloroglucinol (95%) were purchased from Wuhan ChemFaces Biochemical Co Ltd. (Hubei, China).

### Plant Source and Growing Conditions

Helichrysum plants were Cultivated in regular soil and fertigated with 18-18-18 N—P—K—Mg fertilizer. Plants were grown in the greenhouses of the Weizmann Institute in Rehovot, with natural lighting supplemented with HPS artificial lighting to reach 16 h light per day.

UPLC-qTOF Analysis of Cannabinoids from Helichrysum and Cannabis Tissues

Fresh samples of six different tissues: young leaves, old leaves, florets and receptacle of flowers, stem, and root were collected from a plant at the flowering stage. Florets and the receptacle of flowers were detached using a scalpel and extracted separately. All the tissues were flash-frozen in liquid N₂ and ground to a fine powder in a mortar. Then, 100 mg of frozen powder from all the plant tissues were extracted with 1 ml ethanol, vortexed, and then sonicated for 20 min, at room temperature. Finally, the extracts were centrifuged for 15 min at 14,000×g and the solvent was filtered through 0.22 μm filters.

Samples were analyzed using a high-resolution ultrahigh-performance liquid chromatography-tandem quadrupole time-of-flight (UPLC-qTOF) system comprised of a UPLC (Waters Acquity) with a diode array detector connected either to a XEVO G2-S QTof (Waters) or to Synapt HDMS (Waters). The chromatographic separation of compounds was performed on a 100 mm×2.1 mm i.d. (internal diameter), 1.7 μm UPLC BEH C18 column (Waters Acquity). The mobile phase consisted of 0.1% formic acid in acetonitrile:water (5:95, v/v; phase A) and 0.1% formic acid in acetonitrile (phase B). The flow rate was 0.3 ml min-1, and the column temperature was kept at 35° C. Cannabinoids were analyzed using a 29 min multistep gradient method: initial conditions were 40% B for 1 min, raised to 100% B until 23 min, held at 100% B for 3.8 min, decreased to 40% B until 27 min, and held at 40% B until 29 min for re-equilibration of the system. Intermediates and glycosylated compounds were analyzed using a 40 min multistep gradient method: from 0% to 28% B over 22 min, raised to 100% B until 36 min, held at 100% B for 2 min, decreased to 0% B until 38.5 min, and held at 40% B until 40 min for re-equilibration of the system. Electrospray ionization (ESI) was used in negative ionization with an m/z range of 50-1,000 Da. Masses of the eluted compounds were detected with the following settings: capillary 1 kV, source temperature 140° C., desolvation temperature 450° C., and desolvation gas flow 800 l h⁻¹. Argon was used as the collision gas. MS/MS experiments were performed in negative ionization mode according to the observed deprotonated masses. The following settings were used: capillary spray of 1 kV; cone voltage of 30 eV; collision energy ramp of 15-50 eV.

### Compounds Purification for NMR Analysis

A total of 86 g of fresh leaves were flash-frozen in liquid N₂ and ground to a fine powder using an electrical grinder, extracted with 600 ml ethanol, sonicated in an ultrasonic bath for 20 min, and agitated in an orbital shaker at 25° C. for 30 min. Next, the supernatant was filtered under pressure, and the ethanol was evaporated using a rotary evaporator at 40° C. and subsequently lyophilized to remove residual water. The final extract was reconstituted in 25 ml acetonitrile and used for either direct purification (following ten times dilution) or pre-fractionation via medium pressure liquid chromatography (MPLC).

MPLC was performed on a Büchi Sepacore System equipped with two C-605 pump modules, a C-620 control unit, a C-660 fraction collector, a C-640 UV photometer (Büchi Labortechnik AG, Switzerland), and a C18 manually packed column. The mobile phase consisted of acetonitrile:water (5:95, v/v; phase A) and acetonitrile (phase B), with the following multistep gradient method: initial conditions were 0% B for 10 min, raised to 99% B until 530 min, and slowly raised to 100% B until 660 min. The flow rate was 15 ml min⁻¹, the injection volume was 15 ml, and the wavelengths used for monitoring the acquisition were: 210, 224, 270, and 350 nm. Fractions of 100 ml were collected throughout the run, giving 99 tubes. The fractions were analyzed by UPLC-qTOF to select specific compounds for purification. The selected fractions were evaporated using a rotary evaporator at 40° C., lyophilized to remove residual water, reconstituted in methanol, and filtered through a 0.22 μm syringe filter.

Purification of compounds was performed on either an Agilent 1290 Infinity II UPLC system equipped with a quaternary pump, autosampler, diode array detector, a Bruker/Spark Prospekt II LC-SPE system (Spark), and an Impact HD UHR-QqTOF MS (Bruker) connected via a Bruker NMR MS Interface (BNMI-HP) (the general instrument setup was according to Jozwiak et al.); or a UPLC system (Waters Acquity) equipped with a binary pump, an autosampler, a fraction manager and a diode array detector. Triggering on both instruments was performed using specific UV wavelengths according to the compound. The mobile phase consisted of 0.1% formic acid in acetonitrile:water (5:95, v/v; phase A) and 0.1% formic acid in acetonitrile (phase B).

The Bruker system method development was performed by acquiring both MS and UV signals. MS spectra were acquired in negative full scan mode between m/z 50 and 1,700. The chromatographic separation was performed using XBridge (BEH C18, 250×4.6 mm i.d., 5 μm; Waters) or Luna (C18, 250×4.6 mm i.d., 5 μm; Phenomenex) HPLC columns, and the conditions were adjusted and optimized for each compound. In this system, the eluent with the compound of interest were mixed with a makeup-flow of 1.8 ml min⁻¹ water and then trapped on solid-phase extraction (SPE) cartridges (10×2 mm Hysphere resin GP cartridges). Each cartridge was loaded four times with the same compound, and approximately 60 cartridges were used for trapping one compound, depending on the concentration of the sample injected. Before NMR measurements, SPE cartridges were dried with a stream of N₂, and the fraction from each cartridge was eluted with a total of 150 μl MeOH into a 96-well plate. Eluents containing the same compound were pooled, dried under a stream of N₂, and stored at −20° C. until NMR analysis.

The chromatographic separation on the Waters instrument was performed on a Luna Phenyl-Hexyl column (150 mm×2 mm i.d., 3 μm; Phenomenex). The flow rate was 0.3 ml min-1, and the column temperature was kept at 35° C. All other conditions were adjusted and optimized according to the sample. The eluent with the compound of interest was collected in 2 ml HPLC vials. Eluents containing the same compound were pooled, dried under a stream of N₂, lyophilized, and stored at −20° C. until NMR analysis.

### NMR Methods

The purified compounds were resuspended in 300 μl of MeOD-D₄, dried under a stream of N₂ to remove traces of 1H from the previous solvent, reconstituted in 70 μl MeOD-D₄ with 0.01% of 3-propionic-2,2,3,3-D₄ acid sodium salt (that was used as an internal chemical shift reference for ¹H and ¹³C spectra) and transferred into 1.7 mm NMR test tubes for structure elucidation. NMR spectra were recorded on a Bruker AVANCE NEO-600 NMR spectrometer equipped with a 5 mm TCI-xyz CryoProbe. All spectra were acquired at 25° C. The structures of the different compounds were determined by one dimensional (1D) NMR spectra, as well as various two-dimensional (2D) NMR spectra: ¹H-¹H Correlation Spectroscopy (COSY), ¹H-¹H Total Correlation Spectroscopy (TOCSY), ¹H-¹H Rotating Frame Nuclear Overhauser Spectroscopy (ROESY), ¹H-¹³C Heteronuclear Single Quantum Coherence (HSQC), and ¹H-¹³C Heteronuclear Multiple Bond Correlation (HMBC) spectra.

One dimensional 1H NMR spectra were acquired using 16,384 data points and a recycling delay of 2.5 s. 2D COSY, TOCSY and ROESY spectra were acquired using 16,384-8,192 (t₂)×400-512 (t₁) data points. 2D TOCSY spectra were acquired using isotropic mixing times of 100-300 ms. T-ROESY spectra were recorded using spin lock pulses of 100-400 ms. 2D HSQC and 2D HMBC spectra were recorded using 4,096 (t₂)×400-512 (t₁) data points. Multiplicity editing HSQC enables differentiating between methyl and methine groups that give rise to positive correlation versus methylene groups that appear as negative peaks. HMBC delay for evolution of long-range couplings was set to observe long-range couplings of J_{H,C}=8 Hz.

¹H and ¹³C chemical shift assignment was based on information derived from all the NMR spectra. Assignment of the protons as axial or equatorial was based on the observed vicinal J couplings; a large value (>10 Hz) indicates axial protons, further supported by correlations observed in ROESY spectra. ¹H-¹³C correlations observed in HMBC spectra are marked by arrows, and ¹H-¹H correlations observed in COSY spectra are shown by dashed lines.

### Absolute Quantification of CBGA

Samples were extracted as previously described. The final volume was diluted ×10,000 times to fit into the linear range of the calibration curve. Injections were performed on a UPLC (Waters) connected to a Triple Quad detector (TQ-S, Waters) in multiple reaction monitoring (MRM) mode. The chromatographic separation was achieved using a similar column and mobile phase as previously described. A short 7 min method was established using the following multistep gradient program: initial conditions were 57% B raised to 85% B until 4 min, raised to 100% B until 4.2 min, held at 100% B until 6 min, decreased to 67% B until 6.2 min, and held at 67% B until 7 min for re-equilibration of the system. A flow rate of 0.6 ml min⁻¹ was used, the column temperature was 40° C., and the injection volume was 1 μl. The instrument was operated in negative mode with a capillary voltage of 1.5 kV, and a cone voltage of 40 V. Absolute quantification of CBGA was performed by external calibration using two different transitions (359.3>191.2, 32 V for quantification; and 359.3>315.4, 21 V for qualification).

### MALDI Imaging

For localization of terpenophenols to individual trichomes, fresh leaves and flowers were embedded with M1 embedding matrix (Thermo Scientific) in Peel-A-Way disposable embedding molds (Peel-A-Way Scientific) and frozen on dry ice. The embedded tissues were transferred to a cryostat (Leica CM3050) and allowed to thermally equilibrate at −17° C. for at least 2 h. The frozen tissues were sliced into 40 μm-thick sections. The sections were thaw mounted onto Superfrost Plus slides (Fisher Scientific), vacuum dried in a desiccator and imaged with a Nikon DS-Ri2 microscope. A TM sprayer (HTX Technologies) was used to coat the plant tissues with 2,5-dihydroxybenzoic acid (DHB; 40 mg ml-1 dissolved in 70% MeOH containing 0.2% trifluoroacetic acid). The nozzle temperature was set at 70° C. and the DHB matrix solution was sprayed for 16 passes over the tissue sections at a linear velocity of 120 cm min⁻¹ with a flow rate of 50 μl min⁻¹. MALDI imaging was performed using a 7 T Solarix FT-ICR (Fourier Transform Ion Cyclotron Resonance) mass spectrometer (Bruker Daltonics). The datasets were collected in positive ion mode using lock mass calibration (DHB matrix peak: [3DHB+H-3H₂O]+, m/z 409.055408) at a frequency of 1 kHz and a laser power of 40%, with 200 laser shots per pixel and 15 or 25 μm pixel size for the sectioned leaves and flowers, respectively. Each mass spectrum was recorded in the range of m/z 150-3,000 in broadband mode with a Time Domain for Acquisition of 1M, providing an estimated resolving power of 115,000 at m/z 400. The acquired spectra were processed using the Flex-Imaging software 4.0 (Bruker Daltonics). The spectra were normalized to root-mean-square intensity and MALDI images were plotted at theoretical m/z±0.005% with pixel interpolation on.

### Trichome Isolation

Young leaves were harvested and soaked in ice-cold, distilled water and then abraded using a BeadBeater machine (Biospec Products, Bartlesville, OK). The polycarbonate chamber was filled with 15 g of plant material, and with half the volume with glass beads (0.5 mm diameter), XAD-4 resin (1 g/g plant material), and ethanol 80% to full volume. Leaves were beaten by 2-4 pulses of operation of 1 min each. This procedure was carried out at 4° C., and after each pulse the chamber was allowed to cool on ice. Following abrasion, the contents of the chamber were first filtered through a kitchen mesh strainer and then through a 100 μm nylon mesh to remove the plant material, glass beads, and XAD-4 resin. The residual plant material and beads were scraped from the mesh and rinsed twice with additional ethanol 80% that was also passed through the 100 μm mesh. The presence of enriched glandular trichome secretory cells was checked by visualization in an inverted optical microscope.

### Genome Sequencing and Assembly of Helichrysum

The genome size of Helichrysum was estimated by flow cytometry. Briefly, nuclei were isolated by chopping young leaf tissue of Helichrysum and tomato (used as known reference) in isolation buffer. The samples were stained with propidium iodide, and at least 10,000 nuclei were analyzed in a flow cytometer, and the ratio of G1 peak means between both samples was calculated. High molecular weight DNA was extracted from young frozen leaves and sent for sequencing in the Genome Center of UC Davis. The DNA quality was checked by TapeStation traces and a Qubit fluorimeter (Thermo Fisher). Sequencing was done in a Pacbio Sequel II platform, and a ˜12-kilobase DNA SMRT bell library was prepared according to the manufacturer's protocol. Three different SMRT 8M cells were used, yielding a total of 57.8 Gb of HiFi data (˜44× haploid coverage). In addition to Pacbio HiFi data, 200M reads of PE 2×150 Illumina Hi-C data were obtained by Phase Genomics. Hifiasm software was used to integrate both Pacbio HiFi and HiC data to produce chromosome-scale and haplotype-resolved assemblies.

Further scaffolding of the primary assembly was performed using the Hi-C data and the SALSA software. Ragtag was used for a final round of ordering using the primary assembly as reference to reach syntenic scaffolds for each haplotype. Visualizations of Hi-C data were performed with Juicer and whole-genome alignments with the pafr package (dwinter.github.io/pafr/). Finally, the assembly was softmasked for repetitive elements using EDTA.

### RNA Sequencing and Genome Annotation of Helichrysum

RNA was extracted from seven tissues: young leaves, old leaves, florets and receptacles of flowers, stems, roots and trichomes. RNA integrity was checked using a TapeStation instrument. Paired-end Illumina libraries were prepared for five of the tissues and sequenced on Illumina HiSeq 3000 instrument (PE 2×150, ˜40M reads per sample). Random sequencing errors were corrected using Rcorrector and uncorrectable reads were removed. Adaptor and quality trimming were performed using TrimGalore! with the following parameters: --length 36 q -5 --stringency 1-e 0.1 (github.com/FelixKrueger/TrimGalore). Ribosomal RNA was filtered by discarding reads mapping to SILVA_132_LSURef and SILVA 138_SSURef non-redundant databases using bowtie2 --very-sensitive-local mode. Fastq quality checks on each of the steps were performed using MultiQC. The remaining reads were pooled and used for genome-guided de novo transcriptome assembly using Trinity. The Iso-Seq data were obtained from four of the tissues and processed using isoseq3 and cDNA Cupcake ToFU pipelines (github.com/Magdoll/cDNA_Cupcake). Fused and unspliced transcripts were removed, and only polyA positive transcripts were kept for a unique set of high-quality isoforms. Iso-Seq and Trinity transcripts were aligned to the assembly using minimap2 and the BAM files were used in the PASA pipeline to generate RNA-based gene model structures. In addition, the novo gene structures were obtained using the software braker2 and the mentioned BAM files as extrinsic training evidence. Finally, ab initio and RNA-based gene models were combined using EvidenceModeler and a final round of PASA pipeline. Gene functional annotation was performed for the predicted mature transcripts using TransDecoder (github.com/TransDecoder/TransDecoder), which take into account HMMER hits against PFAM and BLASTP hits against UniProt databases for similarity retention criteria. Further annotation of protein-coding transcripts was performed by BLASTP searches against curated plant protein databases and GO and KEGG terms were obtained with Triannotate.

UMI-based 3′ RNAseq of three replicates of the seven tissues was obtained similarly as described. Adaptor and quality trimming were performed using TrimGalore! in two steps, including PolyA trimming mode. Reads were mapped to the genome using STAR, UMI-deduplicated using umitools, and counts were obtained with featureCounts. Normalization was performed with the varianceStabilizingTransformation algorithm of DESeq2, and the CEMItools package was used for coexpression analysis (dissimilarity threshold of 0.6, pvalue of 0.1). Genes in modules with expression profiles in concordance with the metabolites of interest were analyzed. Candidate genes were selected based on functional annotations, and blast hits with known UGT enzymes.

UGT Expression in E. coli BL21 (DE3) Cells and Protein Purification

Selected UGT genes from Helichrysum were individually cloned into the pET28b vector and expressed in E. coli BL21 (DE3) cells. Bacterial starters were grown overnight in LB medium at 37° C., diluted in fresh LB 1:100, and re-incubated at 37° C. When cultures reached A600=0.6, protein expression was induced with 400 μM of isopropyl-1-thio-β-d-galactopyranoside (IPTG) overnight at 15° C. Bacterial cells were lysed by sonication in 50 mM Tris-HCl pH 8, 0.5 mM phenylmethylsulfonyl fluoride (PMSF, Sigma Aldrich) solution in isopropanol, 10% glycerol and protease inhibitor cocktail (Sigma Aldrich), and 1 mg ml-1 lysozyme (Sigma Aldrich). The whole-cell extract was either kept for functional activity or used for protein purification. Purification of proteins was performed on Ni-NTA agarose beads (Adar Biotech). The proteins were eluted with 200 mM imidazole (Fluka) in buffer containing 50 mM NaH₂PO₄, pH 8 and 0.5 M NaCl. Protein concentration of the eluted fractions was measured with Pierce™ 660 nm protein assay reagent (Thermo Scientific).

### β-Glucosidase Assay for Preparation of DHSA

Two MPLC fractions (50 ml each) containing Glc-OA and Glc-DHSA were evaporated as previously described and reconstituted each in 15 ml Mcllvaine buffer (pH 5.0). Reactions were performed in separate 20 ml vials incubated at 45° C. for 24 h. Each reaction consisted of 6 ml of Mcilvaine buffer (pH 5.0), 3 ml of 0.1 mg ml-1 of an almond β-glucosidase solution in Mcllvaine buffer (≥6 U mg-1, Sigma Aldrich), and 1.5 ml of the fractions containing Glc-DHSA. The compounds were extracted using 3 volumes of ethyl acetate:diethyl ether 1:1, evaporated using a rotary evaporator and reconstituted in 5 ml methanol. The products from the reaction contained a mixture of both glucosylated and deglucosylated OA and DHSA. DHSA was therefore purified using the Waters instrument as previously described and reconstituted in 100 μl methanol for the enzymatic assay. The purified DHSA was analyzed via UPLC-qTOF to verify that the purified fraction did not contain Glc-DHSA.

### UGT Enzyme Assay

Recombinant UGT assays using different aromatic substrates were performed by mixing 1.5 μl of the UDP solution (80 mM, final concentration: 2.5 mM), 27.5 μl Tris buffer (100 mM), 1 μl of each of the substrates (50 mM, final concentration: 1 mM) and 20 μl of the lysate enzyme solution. The reactions were incubated at 30° C. for 1 h. To stop the reactions, 50 μl methanol were added to each tube, vortexed for 10 s, centrifuged at maximum speed for 10 min, and then the supernatant was recovered and used for UPLC-qTOF analysis. The assay with the purified UGTs was performed by mixing 2 μl of the cannabinoid acceptors (OA, DHSA, CBGA, heliCBGA, CBDA, A9-THCA, CBCA, olivetol, CBG, CBD or 49-THC, hexanoylphloroglucinol, naringenin chalcone or pinocembrin chalcone) in the presence of 1.5 μl UDP 80 mM, 46.5 μl Tris buffer (100 mM, pH 8.0) and 1 μl of each enzyme. To stop the reactions, 100 μl methanol was added to each tube, and the compounds were extracted and analyzed as previously described. Kinetics assays were performed with the purified enzymes (1.5 μg/μl) dissolved in 45 μl Tris buffer (100 mM, pH 8.0) and substrates were added using varying (0.5 μM-3 mM) and constant (1 mM) concentrations of OA and UDP and the total reaction volume was 50 μl. To stop the reactions, 100 μl methanol was added to each tube, and the compounds were extracted and analyzed as previously described.

### Example 1

### UPLC-qTOF Profiling and RNA-Seq Transcriptome of Helichrysum Tissues

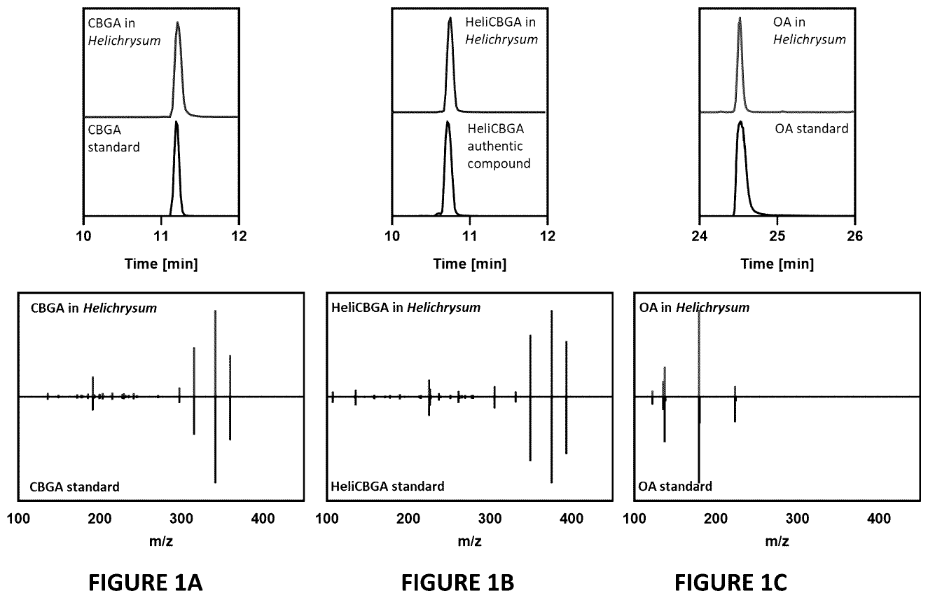

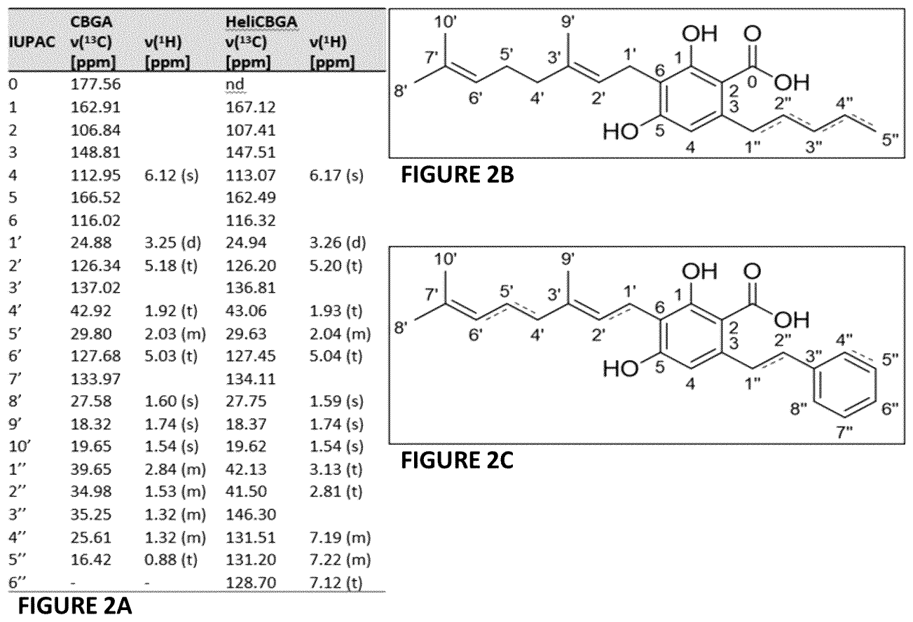

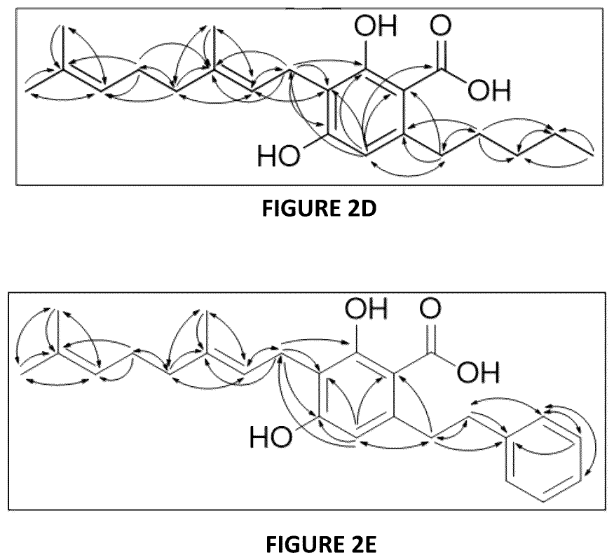

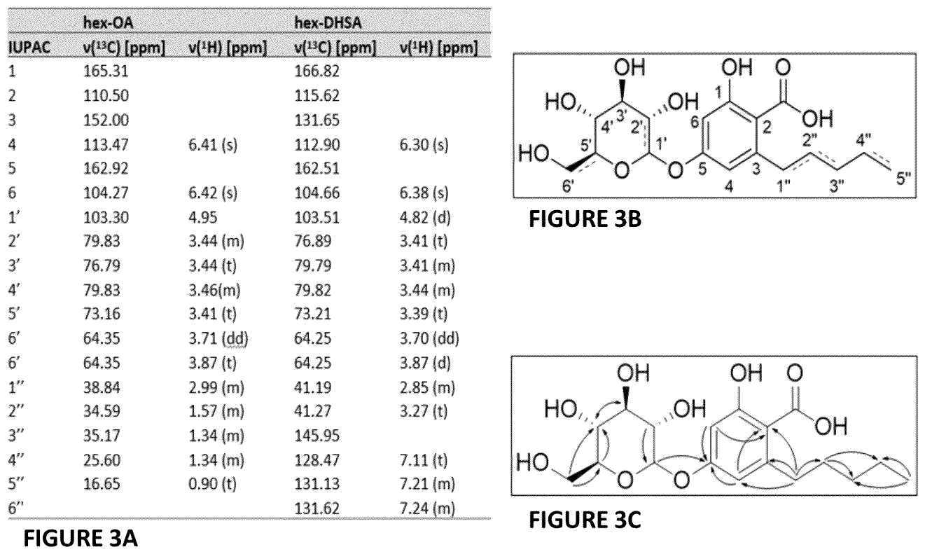

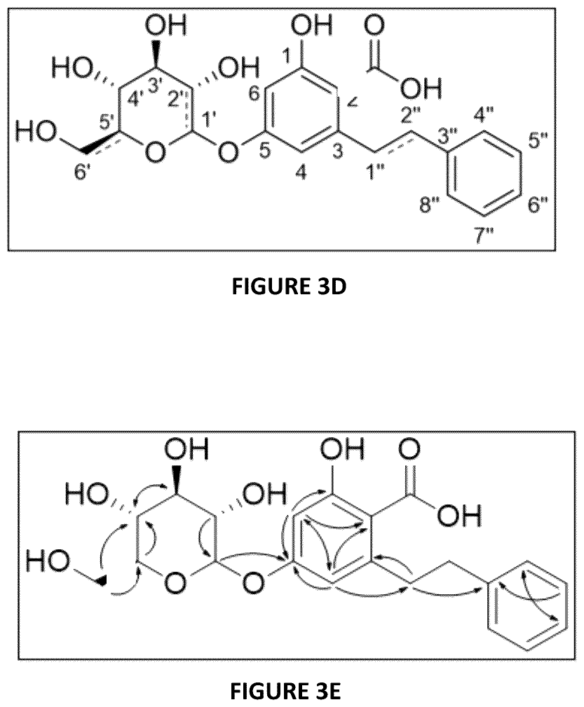

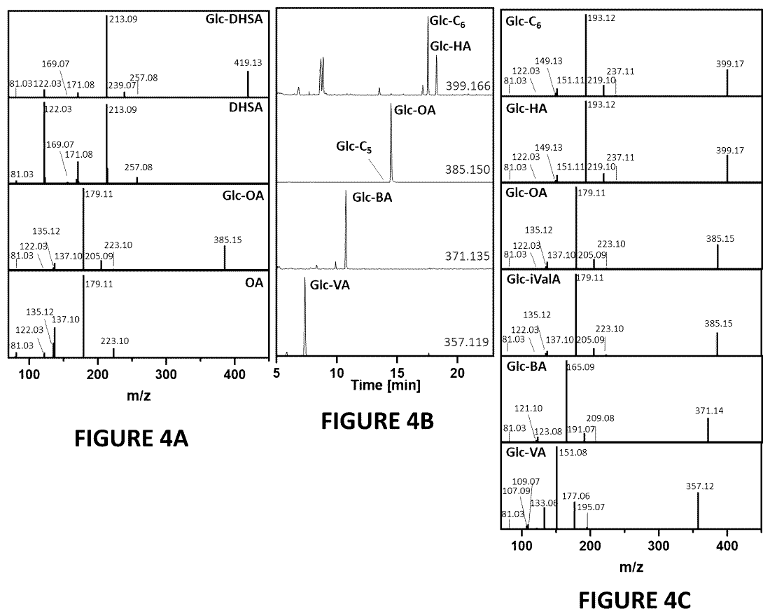

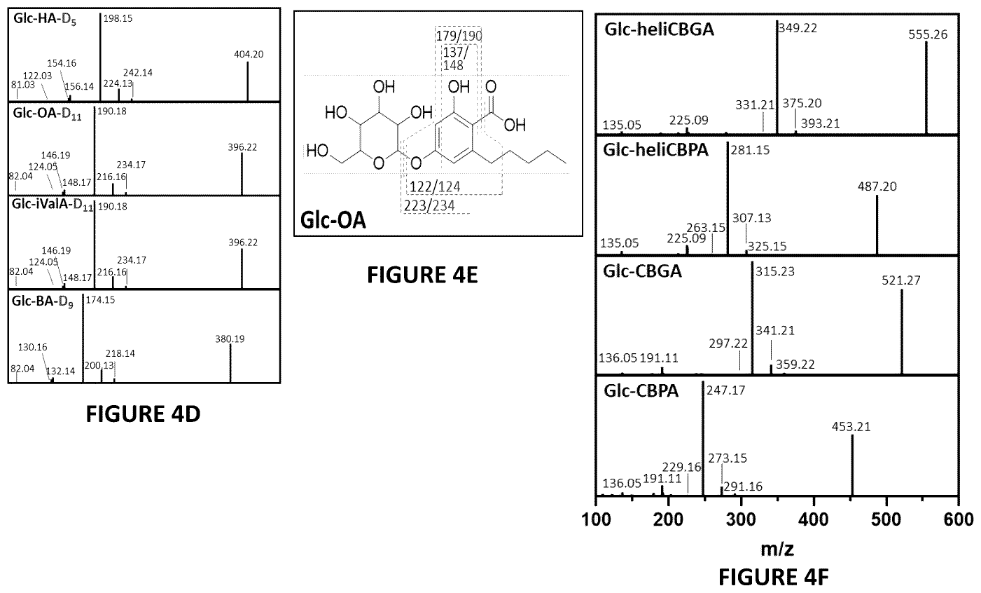

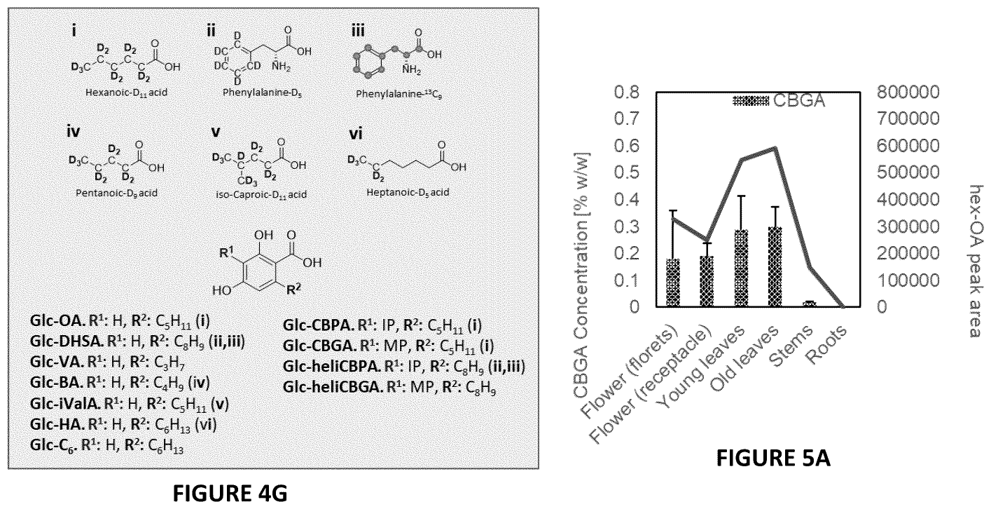

First, the inventors profiled six tissues (young leaf, old leaf, florets, and receptacle of flowers, stem, and root) of Helichrysum using UPLC-qTOF. CBGA and its phenethyl analog heliCBGA were observed in all the tissues besides roots. These compounds were identified by comparison to analytical standards or authentic compounds (FIGS. 1A-1B). According to the metabolic pathways of cannabinoids and amorfrutins in other plants, CBGA and heliCBGA are biosynthesized from the intermediates OA and dihydrostilbenic acid (DHSA), respectively. These compounds and their glycosylated forms (Glc-OA and Glc-DHSA, respectively) were also identified in the Helichrysum extracts according to UPLC-qTOF. OA was identified by comparison with its analytical standard (FIG. 1C) and DHSA by MS/MS spectra and relative retention time (RT) in relation to OA. To confirm the assignment, CBGA, heliCBGA, Glc-OA, and Glc-DHSA were purified and analyzed via one- and two-dimensional nuclear magnetic resonance (NMR) (FIGS. 2-3). Several additional glycosylated compounds were also identified in the Helichrysum extracts via UPLC-qTOF, including C₃-C₆ alkyl-chain intermediates, glycosylated CBGA, and heliCBGA (Glc-CBGA and Glc-heliCBGA, respectively), and the two similar compounds with isoprenyls instead of monoprenyls (Glc-CBPA and Glc-heliCBPA, respectively; FIG. 4). Glycosylated compounds were also observed in Cannabis flowers and leaves, including C₃-C₆ alkyl-chain intermediates, Glc-CBGA, and glycosylated cannabidiolic acid (Glc-CBDA).

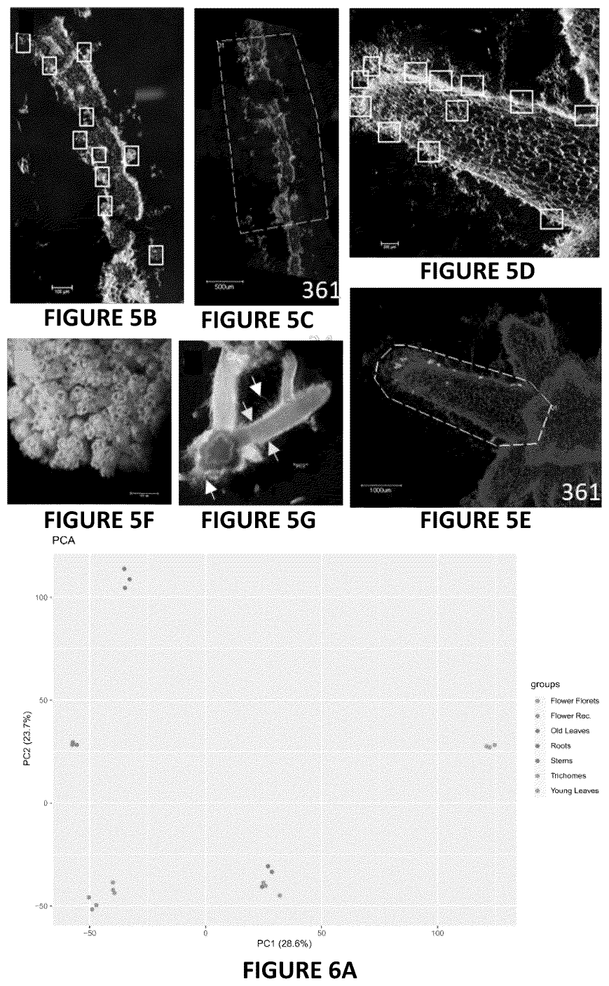

Among the tested tissues in Helichrysum leaves and flowers showed the highest accumulation of CBGA, while roots contained no CBGA (FIG. 5A). A similar trend was also observed for Glc-OA (FIG. 5A). CBGA was further localized to the glandular trichomes of cross-sectioned leaves and flowers via MALDI-MSI (FIGS. 5B-5G). Using this information, RNA-seq transcriptome analysis was performed on these tissues, and candidate genes were selected based on their high expression profile in leaves and isolated trichomes compared to roots.

### Example 2

### Functional Characterization of UGTs

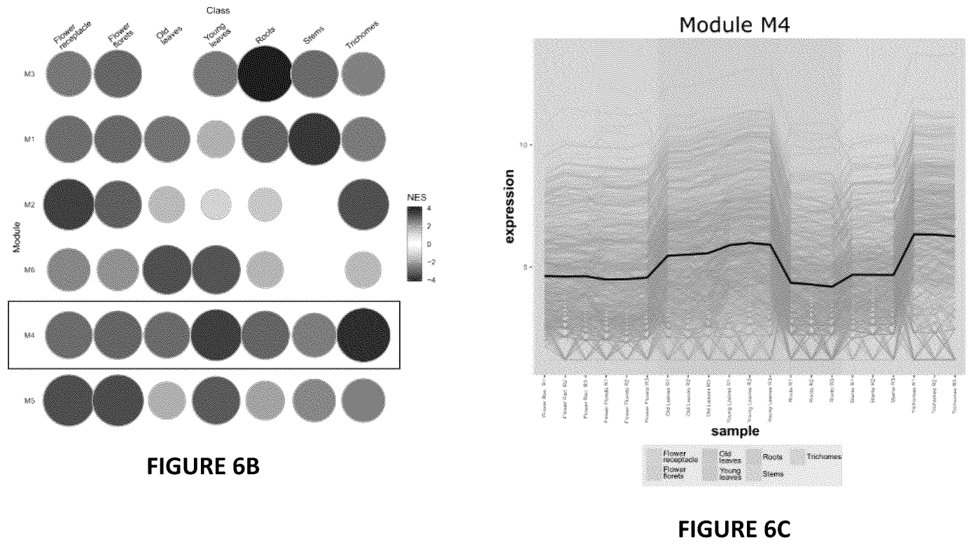

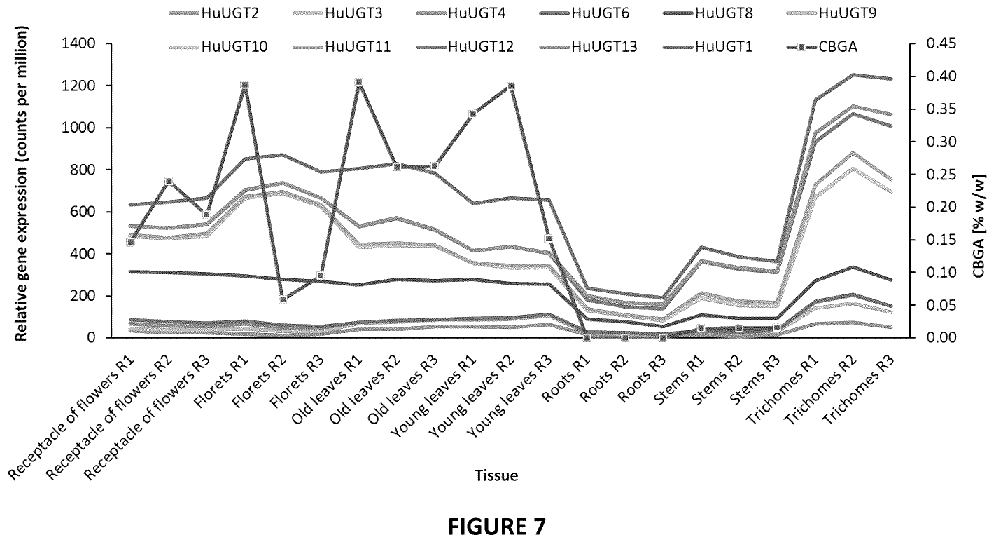

The functional annotation of Helichrysum and Cannabis genes was inferred by sequence similarity with known UGT enzymes. Over 100 genes encoding UGTs have been identified in Arabidopsis thaliana, several of which with established substrate specificities. In particular, the genes encoding the enzymes AtUGT89B1 and AtUGT89A2 were previously found to catalyze the glycosylation of hydroxybenzoic acid (HBA) and several dihydroxybenzoic acids (DHBAs). These molecules are structurally similar to OA and were therefore used to select candidate UGT enzymes in Helichrysum. Additional candidates were identified according to positive correlations between the expression of genes and the accumulation of the metabolites (FIGS. 6-7). Overall, the inventors selected thirteen (HuUGT1-13) candidate genes that encode putative UGTs.

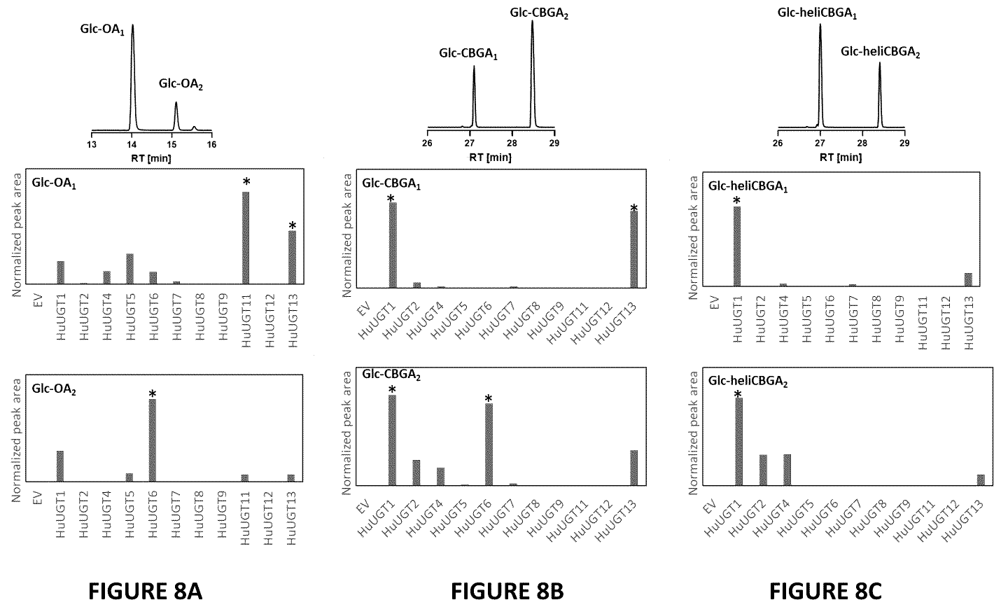

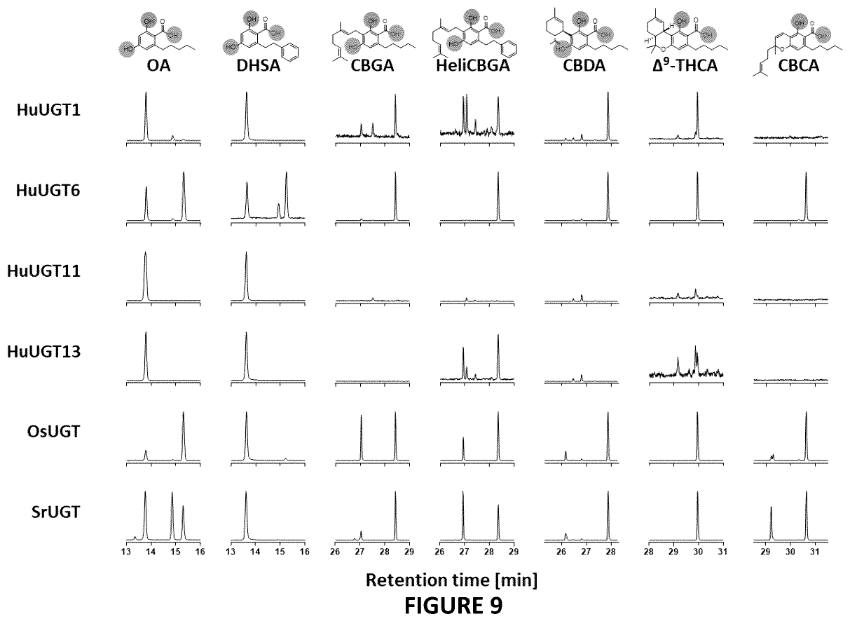

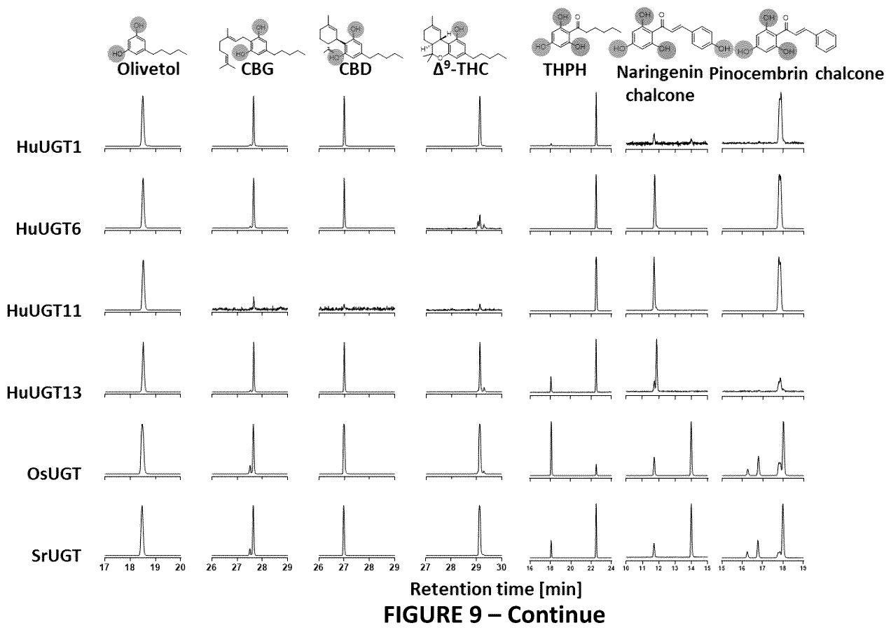

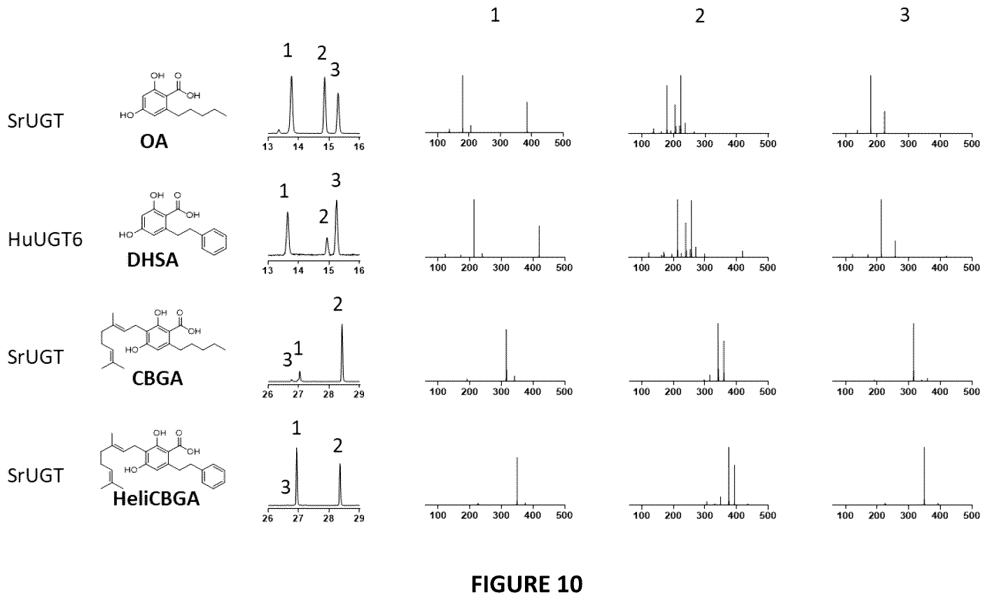

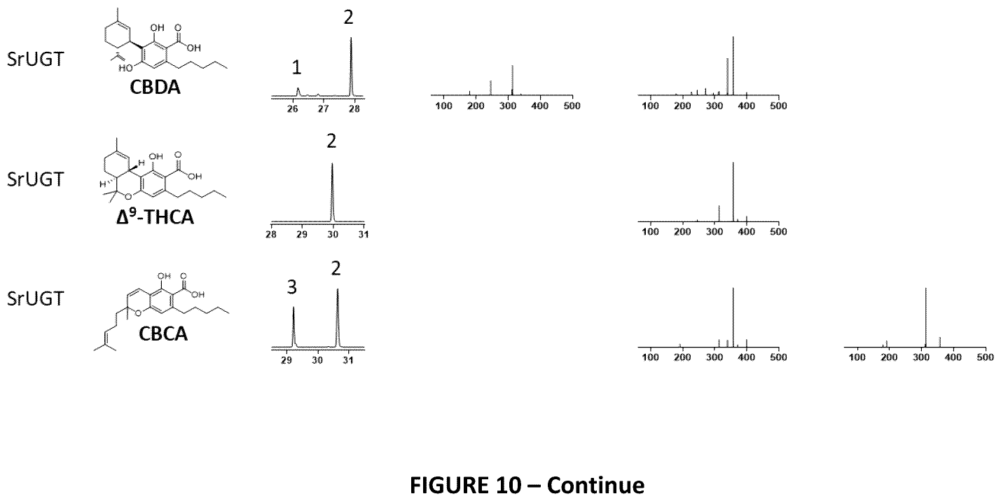

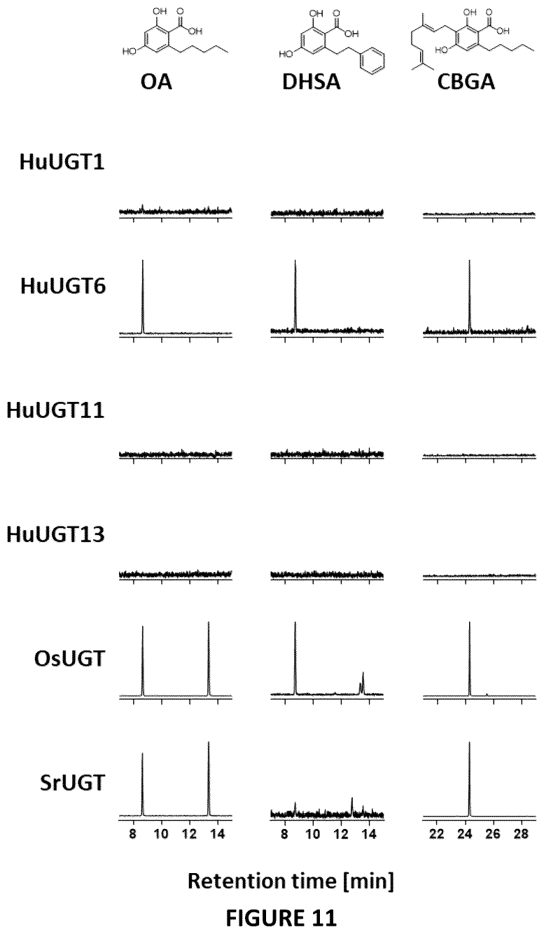

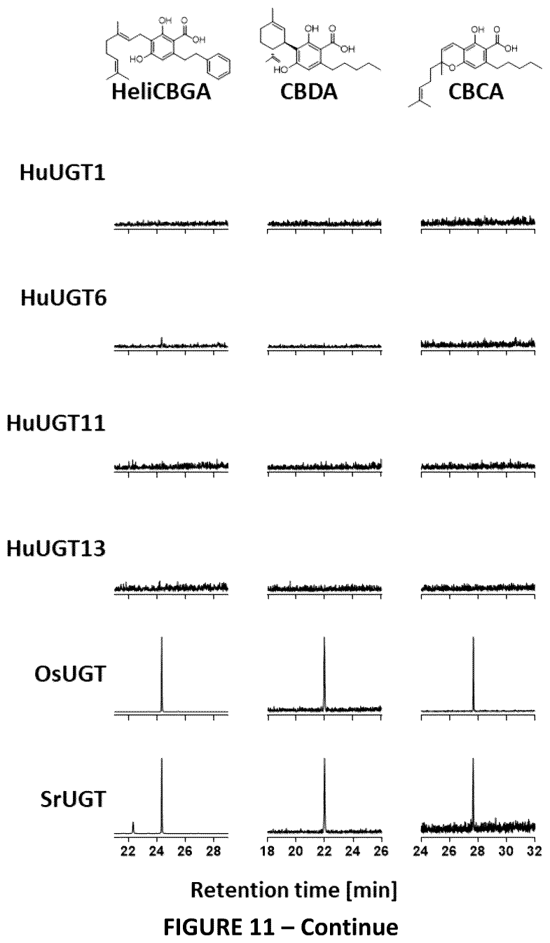

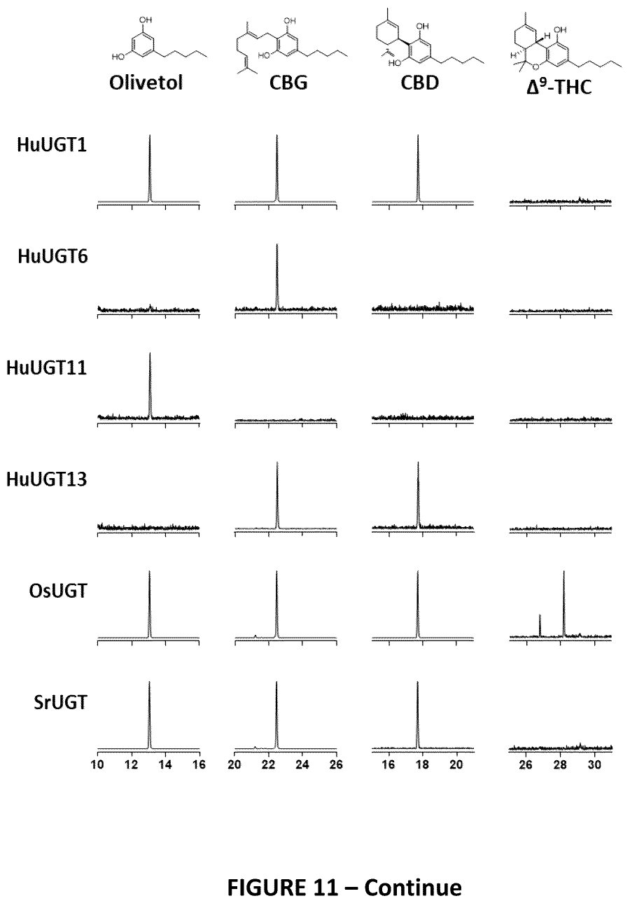

Next, the inventors recombinantly expressed eleven of the thirteen UGTs from Helichrysum in E. coli and used the crude lysate to examine the activity of the proteins using OA, CBGA, and heliCBGA in a reaction including UDP-Glc as the sugar donor. Several enzymes showed activity on the different substrates, including HuUGT1-2 (SEQ ID Nos: 14-15), HuUGT4-7 (SEQ ID Nos: 17-20), HuUGT11 (SEQ ID NO: 24), and HuUGT13 (SEQ ID NO: 26; FIG. 8). The inventors purified the four most active enzymes (HuUGT1 (SEQ ID NO: 14), HuUGT6 (SEQ ID NO: 19), HuUGT11 (SEQ ID NO: 24), and HuUGT13 (SEQ ID NO: 26)) along with the previously characterized enzymes from stevia and rice (SrUGT and OsUGT, respectively), and performed in vitro assays with an array of cannabinoid substrates, both natural and unnatural to Helichrysum (FIG. 9). The inventors screened the LC/HRMS chromatograms for glucosylated and diglucosylated products according to the theoretical m/z values. Two main monoglucosides were observed for each substrate, assigned according to UPLC-qTOF as glucosides, glucosylated on one of the hydroxyls in each molecule (FIGS. 9-10). All the enzymes were active with varying substrate specificity and products produced. For example, HuUGT11 and HuUGT13 were highly active on the cannabinoid intermediates while almost inactive on the prenylated compounds. Diglucosylation of acid compounds was only observed in case of HuUGT6, while olivetol, cannabidiol (CBD) and cannabigerol (CBG) were diglucosylated by different HuUGTs depending on the compound (FIG. 11).

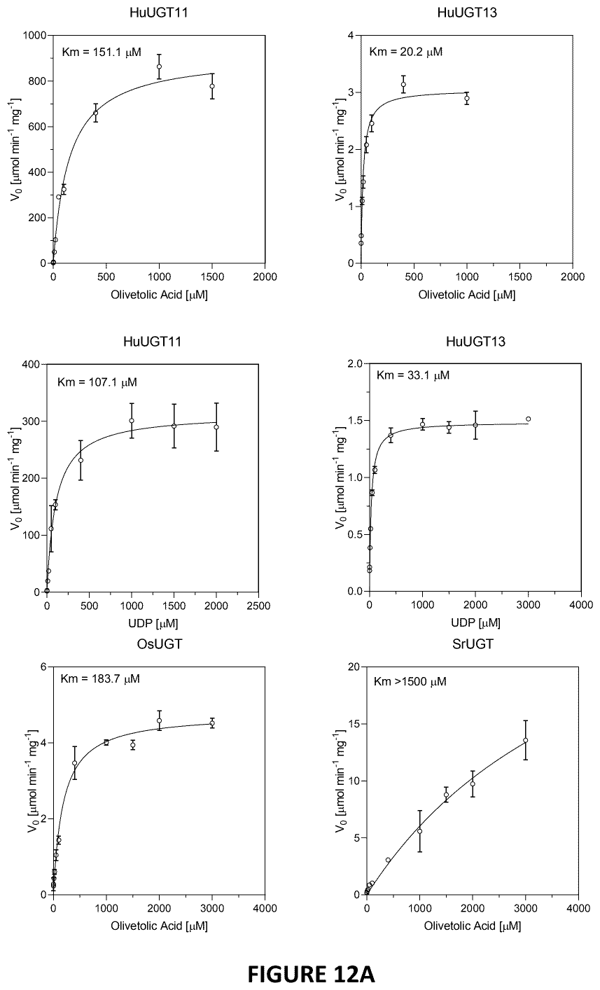

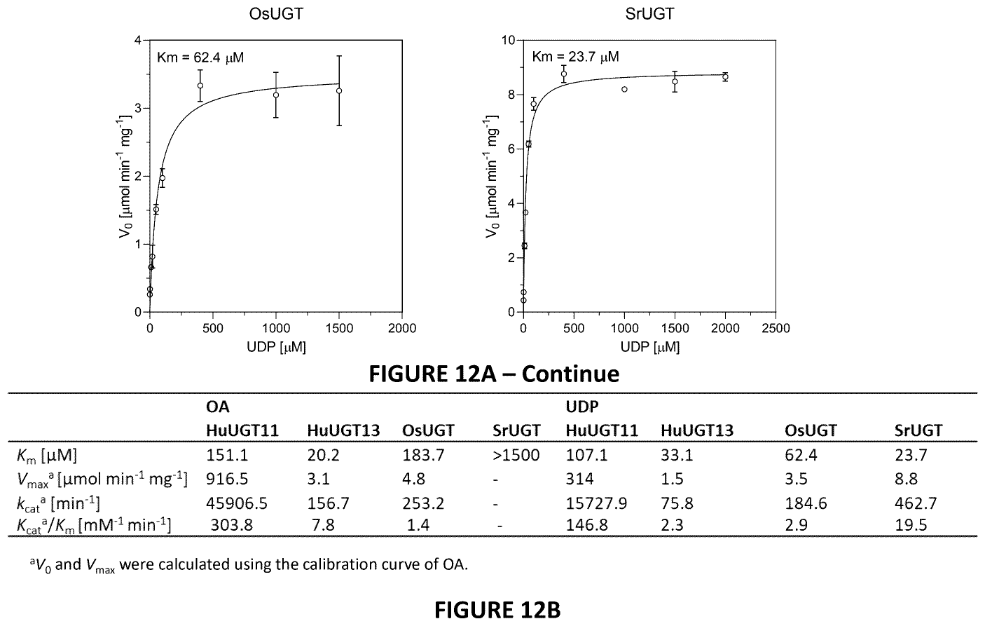

Interestingly, the UGTs from Helichrysum also glucosylated the phloroglucinoid and flavonoid precursors naturally present in the plant (FIG. 9), notwithstanding that the glucosylated forms were not observed in the extracts. Kinetics assays of HuUGT11, HuUGT13, OsUGT, and SrUGT showed an impressive catalytic advantage of HuUGT11 with OA and UDP versus all the other enzymes (FIG. 12). HuUGT11 is therefore a very promising enzyme capable of producing large quantities of Glc-OA and Glc-DHSA, as presented in the plant.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. An isolated DNA molecule comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or any combination thereof.

2. The isolated DNA molecule of claim 1, wherein said nucleic acid sequence having at least 87% homology to any one of SEQ ID Nos.: 1-13 is 700 to 1,800 nucleotides long, and optionally wherein said nucleic acid sequence encodes a protein being a uridine 5′-diphospho (UDP)-glucuronosyltransferase (UGT).

3. (canceled)

4. An artificial nucleic acid molecule comprising the isolated DNA molecule of claim 1.

5. A plasmid or an agrobacterium comprising the artificial nucleic acid molecule of claim 4.

6. An isolated protein encoded by the isolated DNA molecule of claim 1.

7. The isolated protein of claim 6, comprising or consisting of an amino acid sequence with at least 90% homology to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, and optionally wherein said isolated protein is characterized by being capable of glycosylating a cannabinoid or a precursor thereof.

8.-9. (canceled)

10. A transgenic cell comprising the isolated protein of claim 6, optionally wherein said transgenic cell being any one of: a unicellular organism, a cell of a multicellular organism, and a cell in a culture, and optionally wherein said unicellular organism comprises a fungus or a bacterium, and optionally wherein said fungus is a yeast cell.

11.-13. (canceled)

14. An extract derived from the transgenic cell of claim 10, or any fraction thereof, and optionally wherein said extract comprises said isolated protein.

15. (canceled)

16. A transgenic plant, a transgenic plant tissue or a plant part, comprising the isolated protein of claim 6, and optionally wherein said transgenic plant being a Cannabis sativa plant.

17. (canceled)

18. A composition comprising the isolated protein of claim 6, and an acceptable carrier.

19. A method for glycosylating a cannabinoid comprising or a precursor thereof:
a. providing a cell comprising an artificial vector comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13; and
b. culturing said cell from step (a) such that a protein encoded by said artificial vector is expressed,
thereby glycosylating a cannabinoid comprising or a precursor thereof.

20. The method of claim 19, wherein any one of: (i) said cell is a transgenic cell or a cell transfected with an isolated DNA molecule comprising a nucleic acid sequence having at least 87% homology to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, or any combination thereof or an artificial vector comprising thereof; (ii) said protein is characterized by being capable of transferring a glucuronic acid component of UDP-glucuronic acid to said cannabinoid or precursor thereof; (iii) said culturing comprises supplementing said cell with an effective amount of UDP; (iv) said artificial vector is an expression vector; (v) said cell is a prokaryote cell or a eukaryote cell; (vi) said method further comprises a step (c) comprising extracting said cell, thereby obtaining an extract of the cell, and optionally wherein said method further comprising a step preceding step (c), comprises separating said cultured cell from a medium wherein said cell is cultured; (vii) said method further comprising a step preceding step (a), comprising introducing or transfecting said cell with said artificial vector; and (viii) any combination of (i) to (vii).

21.-27. (canceled)

28. An extract of a cell obtained according to (v) to (vii) of the method of claim 20.

29. A medium or a portion thereof separated from a cultured cell, obtained according to the method of claim 20.

30. A composition comprising the extract of claim 28, and an acceptable carrier.

31. A method for glycosylating a cannabinoid or a precursor thereof, the method comprising contacting said cannabinoid or precursor thereof with an effective amount of a protein comprising an amino acid sequence with at least 90% homology to SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, or SEQ ID NO: 26, thereby glycosylating the cannabinoid or a precursor thereof.

32. The method of claim 31, wherein said contacting is in a cell-free system.

33. The method of claim 19, wherein said cannabinoid is CBGA, heliCBGA, CBDA, or any combination thereof.

34. The method of claim 19, wherein said cannabinoid precursor is olivetolic acid (OA).
